SEQUENCE LISTING (47 sequences)

SEQ ID NO: 1 - DNA, 17165 bp, synthetic construct
  source 1..17165: mol_type = other DNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagattaagcgtctgatgagtccgtgaggacgaaacccggcgtaccgggtcacgcttaacaaccagatcaaagaaaaaacagacattgtcaattgcaaagcaaaaatgtaacacccctacaatggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcataacatgaaaaaaactaacacccctcccgtacggccaccatgcacatcagcctgatgtacgccatcctgtgcctgcagctgtgcggcctgggcgagacccacggcagccacaatgagacccggcacaacaagaccgacaccatgaccacccctggcgacaaccccagcagcgagccccctgtgagcaccgccctgagcatcaccctggatcctagcaccgtgacccccaccacccctgccagcggcctggagggcagcggcgaagtgtacaccagcccccccatcaccaccggcagcctgcccctgagcgagaccacccccgagctgcccgtgaccaccggcaccgataccctgagcgccggagatgtggaccccagcacccagacagccggcggaaccagcgcccccacagtgagaaccagcctgcccaatagccctagcaccccaagcacccctcaggacacccaccaccctgtgagaaacctgctgagcgtgaccagccctggccccgacgagaccagcacccccagcggcaccggcaaggagagcagcgccacctccagcccccacccagtgagcaatagaccccctacccctcccgccaccgcccagggccccaccgagaacgacagccacaacgccaccgagcaccccgagagcctgacccagagcgccaccccaggcctgatgaccagcccaacccagatcgtgcacccccagtccgccacccctatcaccgtgcaggatacccaccccagccccaccaacaggagcaagcggaacctgaagatggagatcatcctgaccctgagccagggcctgaagaagtactacggcaagatcctgaggctgctgcagctgaccctggaggaggacaccgagggcctgctggagtggtgcaagagaaacctgggcctggactgcgacgataccttcttccagaagcggatcgaggagttcttcatcaccggcgagggccacttcaatgaagtgctgcagttcagaacccccggcaccctgagcaccaccgagtctacccctgccggcctgcccaccgccgagcccttcaagagctacttcgccaagggcttcctgagcatcgacagcggctactacagcgccaagtgctacagcggcacctccaacagcggactgcagctgatcaacatcacccggcacagcaccagaatcgtggatacccctggccccaagatcaccaacctgaaaaccatcaactgcatcaacctgaaggccagcatcttcaaggagcaccgggaagtggagatcaacgtgctgctgccccaggtggccgtgaatctgagcaactgccacgtggtgatcaagagccatgtgtgcgactacagcctggatatcgacggcgctgtgagactgccccacatctaccacgagggcgtgttcatccctggcacctacaagatcgtgatcgacaagaagaacaagctgaacgaccggtgcaccctgttcaccgactgcgtgatcaagggccgggaagtgagaaagggccagagcgtgctgagacagtacaagaccgagatccggatcggcaaggccagcaccgggtccgaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtaagctagccatgaaaaaaactaacacccctcctttcgaaccatcccaaacatgagcaagatctttgtcaatcctagtgctattagagccggtctggccgatcttgagatggctgaagaaactgttgatctgatcaatagaaatatcgaagacaatcaggctcatctccaaggggaacccatagaggtggacaatctccctgaggatatggggcgacttcacctggatgatggaaaatcgcccaaccatggtgagatagccaaggtgggagaaggcaagtatcgagaggactttcagatggatgaaggagaggatcctagcttcctgttccagtcatacctggaaaatgttggagtccaaatagtcagacaaatgaggtcaggagagagatttctcaagatatggtcacagaccgtagaagagattatatcctatgtcgcggtcaactttcccaaccctccaggaaagtcttcagaggataaatcaacccagactactggccgagagctcaagaaggagacaacacccactccttctcagagagaaagccaatcatcgaaagccaggatggcggctcaaattgcttctggccctccagcccttgaatggtcggctaccaatgaagaggatgatctatcagtggaggctgagatcgctcaccagattgcagaaagtttctccaaaaaatataagtttccctctcgatcctcagggatactcttgtataattttgagcaattgaaaatgaaccttgatgatatagttaaagaggcaaaaaatgtaccaggtgtgacccgtttagcccatgacgggtccaaactccccctaagatgtgtactgggatgggtcgctttggccaactctaagaaattccagttgttagtcgaatccgacaagctgagtaaaatcatgcaagatgacttgaatcgctatacatcttgctaaccgaacctctcccctcagtccctctagacaataaaatccgagatgtcccaaagtcaacatgaaaaaaacaggcaacaccactgataaaatgaacctcctacgtaagatagtgaaaaaccgcagggacgaggacactcaaaaatcctctcccgcgtcagcccctctggatgacgatgacttgtggcttccaccccctgaatacgtcccgctgaaagaacttacaggcaagaagaacatgaggaacttttgtatcaacggaagggttaaagtgtgtagcccgaatggttactcgttcaggatcctgcggcacattctgaaatcattcgacgagatatattctgggaatcataggatgatcgggttagtcaaagtggttattggactggctttgtcaggatctccagtccctgagggcctgaactgggtatacaaattgaggagaacctttatcttccagtgggctgattccaggggccctcttgaaggggaggagttggaatactctcaggagatcacttgggatgatgatactgagttcgtcggattgcaaataagagtgattgcaaaacagtgtcatatccagggcagagtctggtgtatcaacatgaacccgagagcatgtcaactatggtctgacatgtctcttcagacacaaaggtccgaagaggacaaagattcctctctgcttctagaataatcagattatatcccgcaaatttatcacttgtttacctctggaggagagaacatatgggctcaactccaacccttgggagcaatataacaaaaaacatgttatggtgccattaaaccgctgcatttcatcaaagtcaagttgattacctttacattttgatcctcttggatgtgaaaaaaactattaacatccctcaaaagaccccgggaaagatggttcctcaggctctcctgtttgtaccccttctggtttttccattgtgttttgggaaattccctatttacacgataccagacaagcttggtccctggagtccgattgacatacatcacctcagctgcccaaacaatttggtagtggaggacgaaggatgcaccaacctgtcagggttctcctacatggaacttaaagttggatacatcttagccataaaagtgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtctcgatgcaaacatcaaatgaaaccaaatggtgccctcccgataagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcgagacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtaattaattaacgtcctttcaacgatccaagtccatgaaaaaaactaacacccctcccgtacctagcttataaagtgctgggtcatctaagcttttcagtcgagaaaaaaacattagatcagaagaacaactggcaacacttctcaacctgagacttacttcaagatgctcgatcctggagaggtctatgatgaccctattgacccaatcgagttagaggctgaacccagaggaacccccattgtccccaacatcttgaggaactctgactacaatctcaactctcctttgatagaagatcctgctagactaatgttagaatggttaaaaacagggaatagaccttatcggatgactctaacagacaattgctccaggtctttcagagttttgaaagattatttcaagaaggtagatttgggttctctcaaggtgggcggaatggctgcacagtcaatgatttctctctggttatatggtgcccactctgaatccaacaggagccggagatgtataacagacttggcccatttctattccaagtcgtcccccatagagaagctgttgaatctcacgctaggaaatagagggctgagaatccccccagagggagtgttaagttgccttgagagggttgattatgataatgcatttggaaggtatcttgccaacacgtattcctcttacttgttcttccatgtaatcaccttatacatgaacgccctagactgggatgaagaaaagaccatcctagcattatggaaagatttaacctcagtggacatcgggaaggacttggtaaagttcaaagaccaaatatggggactgctgatcgtgacaaaggactttgtttactcccaaagttccaattgtctttttgacagaaactacacacttatgctaaaagatcttttcttgtctcgcttcaactccttaatggtcttgctctctcccccagagccccgatactcagatgacttgatatctcaactatgccagctgtacattgctggggatcaagtcttgtctatgtgtggaaactccggctatgaagtcatcaaaatattggagccatatgtcgtgaatagtttagtccagagagcagaaaagtttaggcctctcattcattccttgggagactttcctgtatttataaaagacaaggtaagtcaacttgaagagacgttcggtccctgtgcaagaaggttctttagggctctggatcaattcgacaacatacatgacttggtttttgtgtttggctgttacaggcattgggggcacccatatatagattatcgaaagggtctgtcaaaactatatgatcaggttcaccttaaaaaaatgatagataagtcctaccaggagtgcttagcaagcgacctagccaggaggatccttagatggggttttgataagtactccaagtggtatctggattcaagattcctagcccgagaccaccccttgactccttatatcaaaacccaaacatggccacccaaacatattgtagacttggtgggggatacatggcacaagctcccgatcacgcagatctttgagattcctgaatcaatggatccgtcagaaatattggatgacaaatcacattctttcaccagaacgagactagcttcttggctgtcagaaaaccgaggggggcctgttcctagcgaaaaagttattatcacggccctgtctaagccgcctgtcaatccccgagagtttctgaggtctatagacctcggaggattgccagatgaagacttgataattggcctcaagccaaaggaacgggaattgaagattgaaggtcgattctttgctctaatgtcatggaatctaagattgtattttgtcatcactgaaaaactcttggccaactacatcttgccactttttgacgcgctgactatgacagacaacctgaacaaggtgtttaaaaagctgatcgacagggtcaccgggcaagggcttttggactattcaagggtcacatatgcatttcacctggactatgaaaagtggaacaaccatcaaagattagagtcaacagaggatgtattttctgtcctagatcaagtgtttggattgaagagagtgttttctagaacacacgagttttttcaaaaggcctggatctattattcagacagatcagacctcatcgggttacgggaggatcaaatatactgcttagatgcgtccaacggcccaacctgttggaatggccaggatggcgggctagaaggcttacggcagaagggctggagtctagtcagcttattgatgatagatagagaatctcaaatcaggaacacaagaaccaaaatactagctcaaggagacaaccaggttttatgtccgacatacatgttgtcgccagggctatctcaagaggggctcctctatgaattggagagaatatcaaggaatgcactttcgatatacagagccgtcgaggaaggggcatctaagctagggctgatcatcaagaaagaagagaccatgtgtagttatgacttcctcatctatggaaaaacccctttgtttagaggtaacatattggtgcctgagtccaaaagatgggccagagtctcttgcgtctctaatgaccaaatagtcaacctcgccaatataatgtcgacagtgtccaccaatgcgctaacagtggcacaacactctcaatctttgatcaaaccgatgagggattttctgctcatgtcagtacaggcagtctttcactacctgctatttagcccaatcttaaagggaagagtttacaagattctgagcgctgaaggggagagctttctcctagccatgtcaaggataatctatctagatccttctttgggagggatatctggaatgtccctcggaagattccatatacgacagttctcagaccctgtctctgaagggttatccttctggagagagatctggttaagctcccaagagtcctggattcacgcgttgtgtcaagaggctggaaacccagatcttggagagagaacactcgagagcttcactcgccttctagaagatccgaccaccttaaatatcagaggaggggccagtcctaccattctactcaaggatgcaatcagaaaggctttatatgacgaggtggacaaggtggaaaattcagagtttcgagaggcaatcctgttgtccaagacccatagagataattttatactcttcttaatatctgttgagcctctgtttcctcgatttctcagtgagctattcagttcgtcttttttgggaatccccgagtcaatcattggattgatacaaaactcccgaacgataagaaggcagtttagaaagagtctctcaaaaactttagaagaatccttctacaactcagagatccacgggattagtcggatgacccagacacctcagagggttgggggggtgtggccttgctcttcagagagggcagatctacttagggagatctcttggggaagaaaagtggtaggcacgacagttcctcacccttctgagatgttgggattacttcccaagtcctctatttcttgcacttgtggagcaacaggaggaggcaatcctagagtttctgtatcagtactcccgtcctttgatcagtcatttttttcacgaggccccctaaagggatacttgggctcgtccacctctatgtcgacccagctattccatgcatgggaaaaagtcactaatgttcatgtggtgaagagagctctatcgttaaaagaatctataaactggttcattactagagattccaacttggctcaagctctaattaggaacattatgtctctgacaggccctgatttccctctagaggaggcccctgtcttcaaaaggacggggtcagccttgcataggttcaagtctgccagatacagcgaaggagggtattcttctgtctgcccgaacctcctctctcatatttctgttagtacagacaccatgtctgatttgacccaagacgggaagaactacgatttcatgttccagccattgatgctttatgcacagacatggacatcagagctggtacagagagacacaaggctaagagactctacgtttcattggcacctccgatgcaacaggtgtgtgagacccattgacgacgtgaccctggagacctctcagatcttcgagtttccggatgtgtcgaaaagaatatccagaatggtttctggggctgtgcctcacttccagaggcttcccgatatccgtctgagaccaggagattttgaatctctaagcggtagagaaaagtctcaccatatcggatcagctcaggggctcttatactcaatcttagtggcaattcacgactcaggatacaatgatggaaccatcttccctgtcaacatatacggcaaggtttcccctagagactatttgagagggctcgcaaggggagtattgataggatcctcgatttgcttcttgacaagaatgacaaatatcaatattaatagacctcttgaattggtctcaggggtaatctcatatattctcctgaggctagataaccatccctccttgtacataatgctcagagaaccgtctcttagaggagagatattttctatccctcagaaaatccccgccgcttatccaaccactatgaaagaaggcaacagatcaatcttgtgttatctccaacatgtgctacgctatgagcgagagataatcacggcgtctccagagaatgactggctatggatcttttcagactttagaagtgccaaaatgacgtacctatccctcattacttaccagtctcatcttctactccagagggttgagagaaacctatctaagagtatgagagataacctgcgacaattgagttctttgatgaggcaggtgctgggcgggcacggagaagataccttagagtcagacgacaacattcaacgactgctaaaagactctttacgaaggacaagatgggtggatcaagaggtgcgccatgcagctagaaccatgactggagattacagccccaacaagaaggtgtcccgtaaggtaggatgttcagaatgggtctgctctgctcaacaggttgcagtctctacctcagcaaacccggcccctgtctcggagcttgacataagggccctctctaagaggttccagaaccctttgatctcgggcttgagagtggttcagtgggcaaccggtgctcattataagcttaagcctattctagatgatctcaatgttttcccatctctctgccttgtagttggggacgggtcaggggggatatcaagggcagtcctcaacatgtttccagatgccaagcttgtgttcaacagtcttttagaggtgaatgacctgatggcttccggaacacatccactgcctccttcagcaatcatgaggggaggaaatgatatcgtctccagagtgatagatcttgactcaatctgggaaaaaccgtccgacttgagaaacttggcaacctggaaatacttccagtcagtccaaaagcaggtcaacatgtcctatgacctcattatttgcgatgcagaagttactgacattgcatctatcaaccggatcaccctgttaatgtccgattttgcattgtctatagatggaccactctatttggtcttcaaaacttatgggactatgctagtaaatccaaactacaaggctattcaacacctgtcaagagcgttcccctcggtcacagggtttatcacccaagtaacttcgtctttttcatctgagctctacctccgattctccaaacgagggaagtttttcagagatgctgagtacttgacctcttccacccttcgagaaatgagccttgtgttattcaattgtagcagccccaagagtgagatgcagagagctcgttccttgaactatcaggatcttgtgagaggatttcctgaagaaatcatatcaaatccttacaatgagatgatcataactctgattgacagtgatgtagaatcttttctagtccacaagatggttgatgatcttgagttacagaggggaactctgtctaaagtggctatcattatagccatcatgatagttttctccaacagagtcttcaacgtttccaaacccctaactgacccctcgttctatccaccgtctgatcccaaaatcctgaggcacttcaacatatgttgcagtactatgatgtatctatctactgctttaggtgacgtccctagcttcgcaagacttcacgacctgtataacagacctataacttattacttcagaaagcaagtcattcgagggaacgtttatctatcttggagttggtccaacgacacctcagtgttcaaaagggtagcctgtaattctagcctgagtctgtcatctcactggatcaggttgatttacaagatagtgaagactaccagactcgttggcagcatcaaggatctatccagagaagtggaaagacaccttcataggtacaacaggtggatcaccctagaggatatcagatctagatcatccctactagactacagttgcctgtgaaccggatactcctggaagcctgcccatgctaagactcttgtgtgatgtatcttgaaaaaaacaagatcctaaatctgaacctttggttgtttgattgtttttctcatttttgttgtttatttgttaagcgtgggtcggcatggcatctccacctcctcgcggtccgacctgggcatccgaaggaggacgcacgtccactcggatggctaagggagagccagaaggatccggctgctaacaaagcccgaaaggaagctgagttggctgctgccaccgctgagcaataactagcataaccccttggggcctctaaacgggtcttgaggggttttttgctgaaagtcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 804 bp, Crimean-Congo hemorrhagic fever virus
  source 1..804: mol_type = other DNA
  misc_feature 1..804: note = Nucleotide sequence encoding GP38
aacctgaagatggagatcatcctgaccctgagccagggcctgaagaagtactacggcaagatcctgaggctgctgcagctgaccctggaggaggacaccgagggcctgctggagtggtgcaagagaaacctgggcctggactgcgacgataccttcttccagaagcggatcgaggagttcttcatcaccggcgagggccacttcaatgaagtgctgcagttcagaacccccggcaccctgagcaccaccgagtctacccctgccggcctgcccaccgccgagcccttcaagagctacttcgccaagggcttcctgagcatcgacagcggctactacagcgccaagtgctacagcggcacctccaacagcggactgcagctgatcaacatcacccggcacagcaccagaatcgtggatacccctggccccaagatcaccaacctgaaaaccatcaactgcatcaacctgaaggccagcatcttcaaggagcaccgggaagtggagatcaacgtgctgctgccccaggtggccgtgaatctgagcaactgccacgtggtgatcaagagccatgtgtgcgactacagcctggatatcgacggcgctgtgagactgccccacatctaccacgagggcgtgttcatccctggcacctacaagatcgtgatcgacaagaagaacaagctgaacgaccggtgcaccctgttcaccgactgcgtgatcaagggccgggaagtgagaaagggccagagcgtgctgagacagtacaagaccgagatccggatcggcaaggccagcaccgggtcc

SEQ ID NO: 3 - protein, 268 aa, Crimean-Congo hemorrhagic fever virus
NLKMEIILTLSQGLKKYYGKILRLLQLTLEEDTEGLLEWCKRNLGLDCDDTFFQKRIEEFFITGEGHFNEVLQFRTPGTLSTTESTPAGLPTAEPFKSYFAKGFLSIDSGYYSAKCYSGTSNSGLQLINITRHSTRIVDTPGPKITNLKTINCINLKASIFKEHREVEINVLLPQVAVNLSNCHVVIKSHVCDYSLDIDGAVRLPHIYHEGVFIPGTYKIVIDKKNKLNDRCTLFTDCVIKGREVRKGQSVLRQYKTEIRIGKASTGS

SEQ ID NO: 4 - DNA, 741 bp, Crimean-Congo hemorrhagic fever virus
  source 1..741: mol_type = genomic DNA
atgcacatcagcctgatgtacgccatcctgtgcctgcagctgtgcggcctgggcgagacccacggcagccacaatgagacccggcacaacaagaccgacaccatgaccacccctggcgacaaccccagcagcgagccccctgtgagcaccgccctgagcatcaccctggatcctagcaccgtgacccccaccacccctgccagcggcctggagggcagcggcgaagtgtacaccagcccccccatcaccaccggcagcctgcccctgagcgagaccacccccgagctgcccgtgaccaccggcaccgataccctgagcgccggagatgtggaccccagcacccagacagccggcggaaccagcgcccccacagtgagaaccagcctgcccaatagccctagcaccccaagcacccctcaggacacccaccaccctgtgagaaacctgctgagcgtgaccagccctggccccgacgagaccagcacccccagcggcaccggcaaggagagcagcgccacctccagcccccacccagtgagcaatagaccccctacccctcccgccaccgcccagggccccaccgagaacgacagccacaacgccaccgagcaccccgagagcctgacccagagcgccaccccaggcctgatgaccagcccaacccagatcgtgcacccccagtccgccacccctatcaccgtgcaggatacccaccccagccccaccaacaggagcaagcgg

SEQ ID NO: 5 - protein, 247 aa, Crimean-Congo hemorrhagic fever virus
MHISLMYAILCLQLCGLGETHGSHNETRHNKTDTMTTPGDNPSSEPPVSTALSITLDPSTVTPTTPASGLEGSGEVYTSPPITTGSLPLSETTPELPVTTGTDTLSAGDVDPSTQTAGGTSAPTVRTSLPNSPSTPSTPQDTHHPVRNLLSVTSPGPDETSTPSGTGKESSATSSPHPVSNRPPTPPATAQGPTENDSHNATEHPESLTQSATPGLMTSPTQIVHPQSATPITVQDTHPSPTNRSKR

SEQ ID NO: 6 - DNA, 35 bp, synthetic construct
  source 1..35: mol_type = other DNA
cgatctgtttacgcgtgccaccatgcacatcagcc

SEQ ID NO: 7 - DNA, 35 bp, synthetic construct
  source 1..35: mol_type = other DNA
agatatcacgctcgaggccaccatggttcctcagg

SEQ ID NO: 8 - DNA, 37 bp, synthetic construct
  source 1..37: mol_type = other DNA
gaagaatctggctagcttacagtctggtctcaccccc

SEQ ID NO: 9 - DNA, 20 bp, synthetic construct
  source 1..20: mol_type = other DNA
ctcgccggtgatgaagaact

SEQ ID NO: 10 - DNA, 20 bp, synthetic construct
  source 1..20: mol_type = other DNA
accctgtgagaaacctgctg

SEQ ID NO: 11 - DNA, 20 bp, synthetic construct
  source 1..20: mol_type = other DNA
ttgatcacgcagtcggtgaa

SEQ ID NO: 12 - DNA, 20 bp, synthetic construct
  source 1..20: mol_type = other DNA
cctgaaggccagcatcttca

SEQ ID NO: 13 - DNA, 20 bp, synthetic construct
  source 1..20: mol_type = other DNA
gcagtaggggcagatgttgt

SEQ ID NO: 14 - DNA, 20 bp, synthetic construct
  source 1..20: mol_type = other DNA
ggcgatatcctggtggactg

SEQ ID NO: 15 - DNA, 19 bp, synthetic construct
  source 1..19: mol_type = other DNA
cagtgtctgcagtaagggc

SEQ ID NO: 16 - DNA, 20 bp, synthetic construct
  source 1..20: mol_type = other DNA
tgcccttactgcagacactg

SEQ ID NO: 17 - DNA, 20 bp, synthetic construct
  source 1..20: mol_type = other DNA
atgtttctgggctcggacag

SEQ ID NO: 18 - DNA, 20 bp, synthetic construct
  source 1..20: mol_type = other DNA
tcaacgtgcagtccacctac

SEQ ID NO: 19 - DNA, 20 bp, synthetic construct
  source 1..20: mol_type = other DNA
tcctcctcgctacagctctt

SEQ ID NO: 20 - DNA, 20 bp, synthetic construct
  source 1..20: mol_type = other DNA
aagagctgtagcgaggagga

SEQ ID NO: 21 - DNA, 24 bp, synthetic construct
  source 1..24: mol_type = other DNA
gctagcttagcctctggttctccg

SEQ ID NO: 22 - DNA, 26 bp, synthetic construct
  source 1..26: mol_type = other DNA
gcggccgcttagcctctggttctccg

SEQ ID NO: 23 - DNA, 18 bp, synthetic construct
  source 1..18: mol_type = other DNA
catagtcatcttcattga

SEQ ID NO: 24 - DNA, 42 bp, synthetic construct
  source 1..42: mol_type = other DNA
gccgccagtgtgctggaattcgccaccatggagacagacaca

SEQ ID NO: 25 - DNA, 32 bp, synthetic construct
  source 1..32: mol_type = other DNA
tcttcaggttgtcaccagtggaacctggaacc

SEQ ID NO: 26 - DNA, 29 bp, synthetic construct
  source 1..29: mol_type = other DNA
cactggtgacaacctgaagatggagatca

SEQ ID NO: 27 - DNA, 39 bp, synthetic construct
  source 1..39: mol_type = other DNA
aacatcgtatggatagtcgacggacccggtgctggcctt

SEQ ID NO: 28 - DNA, 39 bp, synthetic construct
  source 1..39: mol_type = other DNA
taatacgactcactatagggggacagcctgatgacattg

SEQ ID NO: 29 - DNA, 18 bp, synthetic construct
  source 1..18: mol_type = other DNA
tctggtgcatacaaacct

SEQ ID NO: 30 - DNA, 42 bp, synthetic construct
  source 1..42: mol_type = other DNA
caaagaattccggaacgtacggccaccatggagacagacaca

SEQ ID NO: 31 - DNA, 28 bp, synthetic construct
  source 1..28: mol_type = other DNA
ccgaggattcggacccggtgctggcctt

SEQ ID NO: 32 - DNA, 29 bp, synthetic construct
  source 1..29: mol_type = other DNA
caccgggtccgaatcctcggttatccccc

SEQ ID NO: 33 - DNA, 42 bp, synthetic construct
  source 1..42: mol_type = other DNA
gagggaaaaagatctgctagcttacagtctggtctcaccccc

SEQ ID NO: 34 - DNA, 20 bp, synthetic construct
  source 1..20: mol_type = other DNA
cctctgccgacttggcacaa

SEQ ID NO: 35 - DNA, 37 bp, synthetic construct
  source 1..37: mol_type = other DNA
ccggaggattgacgactaatgcaccgccacaaggcag

SEQ ID NO: 36 - DNA, 20 bp, synthetic construct
  source 1..20: mol_type = other DNA
ccgagccattcgaccacatc

SEQ ID NO: 37 - DNA, 44 bp, synthetic construct
  source 1..44: mol_type = other DNA
caaagaattccggaacgtacgatgcacatcagcctgatgtacgc

SEQ ID NO: 38 - DNA, 30 bp, synthetic construct
  source 1..30: mol_type = other DNA
tcttcaggttccgcttgctcctgttggtgg

SEQ ID NO: 39 - DNA, 35 bp, synthetic construct
  source 1..35: mol_type = other DNA
gagcaagcggaacctgaagatggagatcatcctga

SEQ ID NO: 40 - DNA, 34 bp, synthetic construct
  source 1..34: mol_type = other DNA
aaaaagatctgctagcttagagggcgctctggtc

SEQ ID NO: 41 - DNA, 34 bp, synthetic construct; identical to SEQ ID NO: 40
  source 1..34: mol_type = other DNA

SEQ ID NO: 42 - DNA, 28 bp, synthetic construct
  source 1..28: mol_type = other DNA
ggaggtcgactaaagagatctcacatac

SEQ ID NO: 43 - DNA, 29 bp, synthetic construct
  source 1..29: mol_type = other DNA
ttcttcagccatctcaagatcggccagac

SEQ ID NO: 44 - DNA, 23 bp, synthetic construct
  source 1..23: mol_type = other DNA
gttatggtgccattaaaccgctg

SEQ ID NO: 45 - DNA, 20 bp, synthetic construct
  source 1..20: mol_type = other DNA
tctccaggatcgagcatctt

SEQ ID NO: 46 - DNA, 20 bp, synthetic construct
  source 1..20: mol_type = other DNA
gcgtgggtcctggattctat

SEQ ID NO: 47 - DNA, 22 bp, synthetic construct
  source 1..22: mol_type = other DNA
atcgagggaatcggaagagaat

(19) United States
(12) Patent Application Publication
(10) Pub. No.: US 2025/0041398 A1
(43) Pub. Date: Feb. 6, 2025

(54) A THERAPEUTIC AGAINST CRIMEAN-CONGO HEMORRHAGIC FEVER VIRUS

(71) Applicant: Thomas Jefferson University, Philadelphia (PA)

(72) Inventors: Matthias Johannes Schnell, Harleysville, PA (US); Gabrielle Scher, Philadelphia, PA (US)

(21) Appl. No.: 18/719,093

(22) PCT Filed: Dec. 15, 2022
(86) PCT No.: PCT/US22/81661

§ 371 Date: Jun. 12, 2024

Related U.S. Application Data
(60) Provisional application No. 63/290,248, filed on Dec. 16, 2021.

(51) Int. Cl. A61K 39/12; A61K 39/00; A61P 31/14; C07K 14/005; C12N 15/86

(52) U.S. Cl. CPC A61K 39/12; A61P 31/14; C07K 14/005; C12N 15/86; A61K 2039/5252; A61K 2039/575; C12N 2760/12022; C12N 2760/12034; C12N 2760/20143; C12N 2760/20161

(57) ABSTRACT

The present disclosure provides a recombinant vector from an attenuated rabies virus comprising a nucleotide sequence encoding at least one CCHFV glycoprotein and at least one mucin like domain. The disclosure further provides a method for conditioning an immune response with said recombinant virus vector incorporated in said virus virion.

### CROSS-REFERENCE TO RELATED APPLICATIONS

The present application claims priority under 35 U.S.C. § 119 (e) to U.S. Provisional Patent Application No. 63/290,248, filed Dec. 16, 2021, which is herein incorporated by reference in its entirety.

### STATEMENT REGARDING FEDERALLY SPONSORED RESEARCH OR DEVELOPMENT

This invention has been made with government support under grant number T32AI134646 awarded by the National Institutes of Health. The government has certain rights in the invention.

### SEQUENCE LISTING

The XML file named “205961-7079WO1 (00312)_Seq Listing” created on Dec. 8, 2022, comprising 60.9 Kbytes, is herein incorporated by reference in its entirety.

### BACKGROUND OF THE INVENTION

Crimean-Congo hemorrhagic fever virus (CCHFV) causes severe disease in humans with fatality rates reaching 40%. CCHFV is endemic to parts of Africa, Asia, the Middle East, and Europe, specifically to regions in which the tick vector, species of the Hyalomma genus, is present. Classified as an NIH/NIAID Category A and WHO high-priority pathogen, CCHFV poses the highest possible risk to national security and public health. CCHFV is a negative sense single-stranded RNA virus in the order Bunyavirales. Many different animals are susceptible to CCHFV infection, but only humans are known to develop disease. Transmission can occur via tick bite, from animal to human, and from human to human. Given the disease severity and lack of an effective vaccine, CCHFV is classified as a biosafety level 4 (BSL-4) pathogen, which has limited its research and vaccine development. CCHFV is an Emerging Infectious Disease, posing a high risk of a wide-spread outbreak, which without any vaccine or treatment results in global unpreparedness for an outbreak, similar to the current severe acute respiratory syndrome coronavirus 2 outbreak. An inactivated whole virus vaccine was the only CCHFV vaccine to be tested in humans and was ineffective. Accordingly, there is an unmet need for a safe and effective CCHFV vaccine.

### SUMMARY

In some aspects, the present invention is directed to the following non-limiting embodiments:

### Composition

In some aspects, the present invention is directed to a composition.

In some embodiments, the composition includes a recombinant vector from an attenuated rabies virus comprising a nucleotide sequence encoding at least one Crimean-Congo hemorrhagic fever virus (CCHFV) glycoprotein and a mucin-like domain.

In some embodiments, the at least one CCHFV glycoprotein is GP38, or an antigenic fragment thereof

In some embodiments, the nucleotide sequence encoding the recombinant rabies vector has at least 80% sequence identity with SEQ ID NO: 1.

In some embodiments, the nucleotide sequence encoding the recombinant rabies vector has at least 85%, at least 90%, at least 95%, at least 98%, at least 99% or 100% sequence identity with SEQ ID NO: 1.

In some embodiments, the nucleotide sequence encoding GP38 has at least 80% sequence identity with SEQ ID NO: 2.

In some embodiments, the nucleotide sequence encoding GP38 has at least 85%, at least 90%, at least 95%, at least 98%, at least 99% or 100% sequence identity with SEQ ID NO: 2.

In some embodiments, the protein sequence of GP38 has at least 80% sequence identity with SEQ ID NO: 3.

In some embodiments, the protein sequence of GP38 has at least 85%, at least 90%, at least 95%, at least 98%, at least 99% or 100% sequence identity with SEQ ID NO: 3.

In some embodiments, the nucleotide sequence encoding the mucin-like domain has at least 80% sequence identity with SEQ ID NO: 4.

In some embodiments, the nucleotide sequence encoding the mucin-like domain has at least 85%, at least 90%, at least 95%, at least 98%, at least 99% or 100% sequence identity with

In some embodiments, the protein sequence of the mucin-like domain has at least 80% sequence identity with SEQ ID NO: 5.

In some embodiments, the protein sequence of the mucin-like domain has at least 85%, at least 90%, at least 95%, at least 98%, at least 99% or 100% sequence identity with SEQ ID NO: 5.

In some embodiments, the composition is therapeutically effective.

In some embodiments, the composition is immunogenic.

In some embodiments, the composition further includes a pharmaceutically acceptable excipient.

In some embodiments the pharmaceutically acceptable excipient is an adjuvant.

### Isolated Virion

In some aspects, the present invention is directed to an isolated virion.

In some embodiments, the isolated virion prepared from a host cell infected with the recombinant vector the same as or similar to those as described above in the “Composition section”

### Method of Conditioning Immune Response

In some aspects, the present invention is directed to a method of conditioning an immune response protective against a CCHFV virus in a subject.

In some embodiments, the method includes administering to the subject a recombinant vector from an attenuated rabies virus comprising a nucleotide sequence encoding at least one CCHFV glycoprotein and a mucin-like domain.

In some embodiments, the at least one CCHFV glycoprotein is GP38.

In some embodiments, the nucleotide sequence encoding the recombinant rabies vector has at least 80% sequence identity with SEQ ID NO: 1.

In some embodiments, the nucleotide sequence encoding the recombinant rabies vector has at least 85%, at least 90%, at least 95%, at least 98%, at least 99% or 100% sequence identity with SEQ ID NO: 1.

In some embodiments, the nucleotide sequence encoding GP38 has at least 80% sequence identity with SEQ ID NO: 2.

In some embodiments, the nucleotide sequence encoding GP38 has at least 85%, at least 90%, at least 95%, at least 98%, at least 99% or 100% sequence identity with SEQ ID NO: 2.

In some embodiments, the protein sequence of GP38 has a least 80% sequence identity with SEQ ID NO: 3.

In some embodiments, the protein sequence of GP38 has at least 85%, at least 90%, at least 95%, at least 98%, at least 99% or 100% sequence identity with SEQ ID NO: 3.

In some embodiments, the nucleotide sequence encoding the mucin-like domain has at least 80% sequence identity with SEQ ID NO: 4.

In some embodiments, the nucleotide sequence encoding the mucin-like domain has at least 85%, at least 90%, at least 95%, at least 98%, at least 99% or 100% sequence identity with SEQ ID NO: 4.

In some embodiments, the protein sequence of the mucin-like domain has at least 80% sequence identity with SEQ ID NO: 5.

In some embodiments, the protein sequence of the mucin-like domain has at least 85%, at least 90%, at least 95%, at least 98%, at least 99% or 100% sequence identity with SEQ ID NO: 5.

In some embodiments, the composition is therapeutically effective.

In some embodiments, the composition is immunogenic.

In some embodiments, the immune response is directed against a CCHFV glycoprotein.

In some embodiments, the immune response is directed against a CCHFV glycoprotein and a rabies protein.

In some embodiments, the immune response directed against the CCHFV glycoprotein primarily comprises non-neutralizing antibodies.

Method of Treating, Ameliorating and/or Preventing CCHFV Virual Infection

In some aspects, the present invention is directed to a method of treating, ameliorating, and/or preventing CCHFV viral infection in a subject.

In some embodiments, the method includes administering to the subject a composition comprising a recombinant vector from an attenuated rabies virus that expresses at least one CCHFV immunogenic protein and a mucin-like domain, wherein said composition induces an effective immune response against one or both of said viruses wherein the at least one immunogenic CCHFV protein comprises the amino acid sequence of SEQ ID NO: 3.

### CCHFV RNA Polynucleotide Vaccine

In some aspects, the present invention is directed to a CCHFV ribonucleic acid (RNA) polynucleotide vaccine.

In some embodiments, the CCHFV RNA polynucleotide vaccine includes at least one RNA having an open reading frame encoding at least one CCHFV glycoprotein or an immunogenic fragment thereof and a mucin-like domain, and a pharmaceutically acceptable excipient.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A-1B depict a schematic of the various vaccine constructs (FIG. 1A), and the CCHFV glycoproteins incorporated (FIG. 1B). The attenuating R333E mutation in the RABV G region is marked. N, nucleoprotein; P, phosphoprotein; M, matrix protein; G, glycoprotein; L, polymerase; MLD, mucin-like domain; ED51, ectodomain 51 amino acids; TM, transmembrane domain; CD, cytoplasmic domain.

FIG. 2 depicts a Western blot of the characterization of the BNSP333-GP85 Virus construct. Sucrose-purified virions were run on an SDS PAGE protein gel. Viruses analyzed are the parental BNSP333 vector, a recombinant RABV with an irrelevant protein subolesin (SUB) or BNSP333-GP85. The blot was probed with monoclonal antibody 13G8, which is specific for CCHFV GP38.

FIG. 3A-3E depict the GP38 construct and a comparison of the properties of the expressed GP38 and GP85 constructs. FIG. 3A, the BNSP333-GP38 construct, lacking the MLD domain; FIG. 3B, immunoblotting results of the expressed GP38 and GP85 constructs with an anti-GP38 antibody; FIG. 3C, GP38 surface expression in the GP38 and GP85 constructs; FIG. 3D, surface staining for GP38 in cells infected with the GP38 and GP85 constructs; FIG. 3E, intracellular staining for GP38 in cells infected with the GP38 and GP85 constructs.

FIG. 4 depicts immunofluorescence detection of surface antigens of infected cells showing that they express the expected glycoproteins.

FIG. 5A-5D depict SDS-PAGE and immunoblots showing that vaccine viruses express the expected glycoproteins. FIG. 5A, SDS-PAGE of BNSP333, FR1 and GP38+ Gc−; FIG. 5B, SDS-PAGE of VSV-GFP, GP38− Gc+, and GP38+ Gc+; FIG. 5C, immunoblotting of cells infected with the six constructs with anti-GP38; FIG. 5D, immunoblotting of cells infected with the six constructs with anti-Gc.

FIG. 6A-6B depict CCHFV-Gc surface staining (FIG. 6A) and CCHFV-GP38 surface staining (FIG. 6B) in cells infected with the six constructs.

FIG. 7A-7B is a schematic representation of the live in vivo experiment to test vaccine immunogenicity. FIG. 7A, timeline of immunization and booster administered; FIG. 7B, types of vaccines received by groups A-D (FIG. 7B).

FIG. 8 depicts the EC₅₀ titers over time from the live vaccine mouse experiment. Shown are the EC₅₀ titers from total IgG ELISAs against GP38.

FIG. 9A-9B is a schematic representation of the killed vaccine in vivo experiment to test vaccine immunogenicity. FIG. 9A, timeline of immunization and booster administered; FIG. 9B, types of vaccines received by groups A-D (FIG. 9B).

FIG. 10A-B depict the EC₅₀ of the anti GP38 (FIG. 10A) and anti Gc antibody titers (FIG. 10B) in mice vaccinated with GP38+ Gc−, GP38+ Gc+, GP38− Gc+ and Filorab1.

FIG. 11A-11B depict the EC₅₀ values of the anti-GP38 antibody titers (FIG. 11A) and the anti-Gc antibody titers (FIG. 11B) over time.

FIG. 12A-12C depict the EC₅₀ values of the various isotype antibodies induced upon immunization with GP38+ Gc− and GP38+ Gc+. FIG. 12A, IgG2c; FIG. 12B, IgG2b; FIG. 12C, IgG1.

FIG. 13A-13B depict the isotype ratios of IgG2c/IgG1 and IgG2b/IgG1 elicited upon immunization with GP38+ Gc−(FIG. 13A) and GP38+ Gc+ (FIG. 13B)

FIG. 14 depicts the EC₅₀ titers over time from the killed vaccine mouse experiment. Shown are the EC₅₀ titers from total IgG ELISAs against GP38.

FIG. 15A-15D depict the weight curves of challenge experiments showing that virus VΔGcoM is pathogenic in IFNAR−/− mice. FIG. 15A, mice challenged with 5×10⁵ PFU, FIG. 15B, mice challenged with 7.5×10⁵ PFU; FIG. 15C, mice challenged with 1×10⁶ PFU; FIG. 15D, group averages.

FIG. 16 depicts the log viral RNA copy numbers over time, showing that surrogate challenge virus VΔGcoM causes high levels of viremia.

FIG. 17A-17B is a schematic representation showing the time line of immunization, booster and viral challenge (FIG. 17A), and the vaccines received by female and male mice in groups A-E (FIG. 17B).

FIG. 18A-18D depict the EC₅₀ values of the anti-GP38 antibody titers after immunization with constructs as shown in the legends of FIG. 18A-18B, and FIG. 18C-18D. FIG. 18A-18B compares GP38 EC₅₀ values between IFNAR−/− mice vaccinated with the various constructs.

FIG. 18C-18D compares GP38 EC₅₀ values between IFNAR−/− and wildtype mice.

FIG. 19A-19F depict the weight curves of mice challenged. FIG. 19A, mice immunized with BNSP33-GP85 (females), FIG. 19B, mice immunized with BNSP33-GP85 (males); FIG. 19C, mice immunized with Filorab1 (females); FIG. 19D, mice immunized with Filorab1 (males); FIG. 19E, unimmunized naïve B6 mice (wildtype); FIG. 19F, group average weight curves and statistical analysis of comparisons between groups.

FIG. 20 depicts the log viral RNA copy number over in female mice immunized with BNSP33-GP85; male mice immunized with BNSP33-GP85, female mice immunized with Filorab1; male mice immunized with Filorab1, and in unimmunized Naïve B6 mice (wildtype).

FIGS. 21A-21B: CCHFV genome and rhabdoviral-based CCHFV vaccine vector maps, in accordance with some embodiments. Schematics of the CCHFV genome and virion (FIG. 21A), and RABV- and VSV-based CCHFV vaccines and their vector controls (FIG. 21B). All foreign genes were inserted into the BNSP333 vector between N and P and between M and L for the VSV vector. The GP85 chimeric gene is expanded to show the various sections of both GP85 and the RABV-G that were included in the gene. Attenuating R333E mutation is marked in RABV-G. RdRp, RNA-dependent RNA polymerase; MLD, Mucin-like domain; NSM, non-structural M protein; NP, nucleoprotein; N, nucleoprotein; P, Phosphoprotein; M, matrix protein; G, glycoprotein; L, polymerase; ED51, 51 amino acids of the ectodomain;™, transmembrane domain; CD, cytoplasmic domain. Created with Biorender.com.

FIGS. 22A-22I: Rhabdoviral vectors express and incorporate CCHFV glycoproteins, in acordane with some embodiments. Characterization of rhabdoviral-vectored CCHFV vaccines through Immunofluorescence (FIGS. 22A-22B), flow cytometry (FIGS. 22C-22D), SDS PAGE protein gel (FIG. 22E), Western Blot (FIG. 22F), and Growth Curves (FIGS. 22G-22I). Vero E6 cells were infected at MOI 0.01 and fixed after 72 or 24 hrs for RABVs and VSVs, respectively. Cells were stained with α-RABV-G 4C12 (purple) and α-CCHFV-Gc 11E7 (FIG. 22A) or α-CCHFV-GP38 13G8 (FIG. 22B) (red) and mounted with mounting media containing a nuclear DAPI stain (blue). In the merged images, GFP from VSV GFP is green, and areas where there is overlapping expression of RABV-G and the CCHFV glycoproteins are pink. Images were taken at 40× magnification with a 2X zoom. Scale bars represent 10 μm. (FIG. 22C) Vero E6 cells were infected at MOI 10 and fixed after 48 hrs for RABVs or infected at MOI 5 and fixed after 8 hrs for VSVs. Cells were probed for α-RABV-G 4C12 and α-CCHFV-Gc 11E7 or α-CCHFV-GP38 13G8 and analyzed by flow cytometry. Assay was performed multiple times, and the graph is one representative experiment. (FIG. 22D) SDS PAGE protein gel of sucrose purified virions. 1 μg of each virus was loaded onto the gel and all native rhabdoviral proteins and foreign proteins are indicated by the arrows next to each gel. (FIG. 22E) Western blot of sucrose purified virions. 1 μg of each virus was loaded onto the gel and transferred to a nitrocellulose membrane for western blotting. Blots were either probed with α-CCHFV-GP38 13G8 (top panel), α-CCHFV-Gc 11E7 (middle panel) or α-RABV-G 4C12 (bottom panel). (FIG. 22F) Multi-step and one-step growth curves. Cells were infected at MOI 0.01 for multi-step or MOI 10 for one-step growth curves and samples were titered in triplicate. Statistics are differences in titer compared to the parental vector for each growth curve (**** P<0.0001; *** P<0.0002; ** P<0.0021; *P<0.0332).

FIGS. 23A-23H: Rhabdoviral-based CCHFV vaccines elicit humoral responses against respective antigens, in accordance with some embodiments. Immunogenicity study to look at antibody responses induced by each CCHFV vaccine. (FIG. 23A) Immunization and blood draw schedule for mouse studies. Groups of 5 mice were immunized with 10 μg/dose of BPL inactivated vaccines adjuvanted with 5 μg of PHAD in 2% SE per dose. Syringes represent immunizations, red blood drops indicate the days blood was taken and the skull denotes the conclusion of the study when the mice were sacrificed. Created with Biorender.com. (FIG. 23B) Table showing the vaccine groups used in this study and the symbols and colors used to denote each group and assay controls. (FIGS. 23C, 23E, and 23G) Group average ELISA curves for each antigen at the peak of the antibody response. (FIGS. 23D, 23F, and 23H) EC₅₀ ELISA titers over time for each antigen. Error bars indicate the mean with standard deviation (SD) for groups of 5 mice with samples run in duplicate. An ordinary one-way ANOVA with Tukey's Multiple Comparison Test was used to determine statistical differences between groups at each time point. All groups with detectable antibody titers have 4-star significance compared to groups where no antibody titers were detected (**** P<0.0001; *** P<0.0002; ** P<0.0021; * P<0.0332; ns=not significant). (FIGS. 23C and 23D) α-CCHFV-GP38 ELISAs, (FIGS. 23E and 23F) α-CCHFV-Gc ELISAs, and (FIGS. 23G and 23H) α-RABV-G ELISAs. •, mouse 1; ▪, mouse 2; ▴, mouse 3; ▾, mouse 4; ♦, mouse 5.

FIGS. 24A-24D: Rhabdoviral-based CCHFV vaccines induce a Th1-skewed humoral response, in accordance with some embodiments. Isotype subclass ELISAs for each vaccine that had detectable antibodies in the CCHFV glycoprotein IgG Fc ELISAs. (FIGS. 24A and 24C) EC₅₀ antibody titers for each isotype subclass. (FIGS. 24B and 24D) Isotype ratios comparing EC₅₀ titers of IgG2c or IgG2b to IgG1. Any animals with undetectable IgG1 were excluded from isotype ratio calculations. (FIGS. 24A and 24B) GP38 isotype subclass ELISAs. (FIGS. 24C and 24D) Gc isotype subclass ELISAs. Error bars indicate the mean with standard deviation (SD) for groups of 5 mice with samples run in duplicate. Mann Whitney test was used to determine statistical differences between groups for each isotype. (**** P<0.0001; *** P<0.0002; ** P<0.0021; * P<0.0332; ns=not significant).

FIGS. 25A-25F: GP38+ Gc− vaccine is protective in VSV-based surrogate challenge model, in accordance with some embodiments. Challenge study to determine the utility of a VSV-based surrogate challenge virus when looking at vaccine protective efficacy. (FIG. 25A) Experimental timeline. Groups of 10 mice, 5 male and 5 female, were immunized with 10 μg/dose of BPL inactivated vaccines adjuvated with 5 μg of PHAD in 2% SE per dose as indicated by the syringe with the rhabdovirus containing multiple glycoproteins. Challenge of 5E5pfu of surrogate virus is indicated by the syringe with a VSV with a singular set of glycoproteins. Red blood drops indicate the days blood was taken, and the skull denotes the conclusion of the study when any surviving mice were sacrificed. Created with Biorender.com. (FIG. 25B) Table of vaccine groups and representative colors. GP38 EC₅₀ titers pre-challenge (FIG. 25C) and post-challenge (FIG. 25F). Error bars indicate the mean with standard deviation (SD) for groups of 5 mice with samples run in triplicate. (FIG. 25D) Average group weight curves. Error bars indicate SD. (FIG. 25E) Viral RNA copies in the blood as determined by VSV-N qPCR. LOD, limit of detection. Error bars indicate the mean with SD. Results show the combination of two independent experiments; hollow symbols represent the first experiment and symbols with a black outline represent the second experiment. An ordinary one-way ANOVA with Tukey's Multiple Comparison Test was used to determine statistical differences between groups at each time point for EC₅₀ titers and viremia (FIGS. 25C, 25E and 25F). Two-way ANOVA with Tukey's Multiple Comparison Test was used to determine statistical differences between groups for the weight curves (FIG. 25D). All comparisons between groups not listed on the EC₅₀ or weight change graphs had 4-star significant difference. (**** P<0.0001; *** P<0.0002; ** P<0.0021; * P<0.0332; ns=not significant).

FIGS. 26A-26D: Vaccines that incorporate GP38 are protective against WT CCHFV challenge, in accordance with some embodiments. Challenge study to determine rhabdoviral-based CCHFV vaccine protective efficacy against CCHFV. (FIG. 26A) Experimental timeline. Groups of 10 mice, 5 male and 5 female, were immunized with 10 μg/dose of BPL inactivated vaccines adjuvanted with 5 μg of PHAD in 2% SE per dose as indicated by the syringe with the rhabdovirus. As denoted by the syringe with the antibody, mice were given mAb 5A3 24 hrs before and after challenge to make them susceptible to CCHFV. The syringe with the CCHFV indicates when mice were challenged with 1000pfu of strain IbAr10200 I.P. Red blood drops indicate the days blood was taken and the skull denotes the conclusion of the study when any surviving mice were sacrificed. Created with Biorender.com. (FIG. 26B) Table of vaccine groups, the expected outcome for that group and their representative colors. (FIG. 26C) Group average weight change over time. Error bars represent standard deviation. Dotted line indicates weight loss threshold for euthanasia. Statistics are two-way ANOVA compared to female PBS control group (**** P<0.0001). (FIG. 26D) Kaplan-Meyer survival curves. Log-rank Mantel-Cox test was used to determine the significance of survival of each group compared to the female PBS control group (** P<0.0021).

FIGS. 27A-27B. GP38 does not elicit CCHFV neutralizing antibodies, in accordance with some embodiments. CCHFV and RABV neutralization assays. (FIG. 27A) Focus reduction neutralization test (FRNT) of a CCHFV strain IbAr10200 expressing ZsGreen (rCCHFV-ZsGreen) with sera from mice immunized with rhabdoviral vaccines. Hyperimmune mouse ascitic fluid (HMAF) against CCHFV served as a positive control. Error bars represent standard deviation (SD). (FIG. 27B) Rapid fluorescent focus inhibition test (RFFIT) with sera from mice immunized with rhabdoviral vaccines against RABV (strain CVS-11). Graph shows the RABV neutralizing IU/mL values for individual mice. Error bars represent SD. Ordinary one-way ANOVA with Tukey's Multiple Comparison Test was used to determine statistical differences between groups. All groups with detectable RABV neutralizing antibody titers have 4-star significance compared to groups where no antibody titers were detected (**** P<0.0001; *** P<0.0002; ** P<0.0021; * P<0.0332; ns=not significant). Dotted line indicates 0.5IU/mL, the WHO suggested protective threshold. •, mouse 1; ▪, mouse 2; ▴, mouse 3; ▾, mouse 4; ♦, mouse 5.

FIGS. 28A-28C: Gating Strategy and raw data for FIGS. 22C and 22D, in accordance with some embodiments. FIG. 28A: Gating strategy for quantifying antigen expression on the surface of infected cells. FIGS. 28B-28C: Histograms and numerical values of flow cytometry staining of infected cells. Vero E6 cells were infected with RABVs at MOI 10 for 48 hrs or VSVs at MOI 5 for 8 hrs and then fixed. Cells were then probed with α-RABV-G 4C12 and α-CCHFV-Gc 11E7 (FIG. 28B) or α-CCHFV-GP38 13G8 (FIG. 28C) and analyzed by flow cytometry. Experiment was performed multiple times, and this is one representative experiment.

FIGS. 29A-29C: Raw files for FIGS. 22E and 22F, in accordance with some embodiments. FIGS. 29A-29B: SDS PAGE protein gel of sucrose purified virions. 1 μg of sucrose purified virions were run on the gel and stained with SYPRO™ Ruby stain. (FIG. 29A) Gel that was used for RABVs in FIG. 22E. (FIG. 29B) Gel that was used for VSVs in FIG. 22E. (FIG. 29C) Western blot of sucrose purified virions. 1 μg of sucrose purified virions were run on an SDS PAGE gel and transferred to a nitrocellulose membrane for western blotting. Blots were either probed with α-CCHFV-GP38 13G8 (top panel), α-CCHFV-Gc 11E7 (middle panel) or α-RABV-G 4C12 (bottom panel). Image on the left is the merge of both visible and chemiluminescent channels to be able to see the ladder. Image on the right is just the chemiluminescent channel.

FIG. 30A-30D: The Mucin-Like Domain is important for GP38 Processing, in accordance with some embodiments. (FIG. 30A) Schematic of BNSP333-GP38 vaccine construct with chimeric GP38/RABV-G pop out to show the individual domains of the RABV-G tail. Created with Biorender.com. (FIG. 30B) Immunofluorescence staining of infected cells. Vero E6 cells were infected with either BNSP333-GP38 or BNSP333-GP85 at MOI 0.01 for 72 hrs and then fixed. Cells used for Intracellular staining were permeabilized with 0.1% Triton™ X-100 following fixation. Cells were then stained with α-RABV-G 4C12 (purple) and α-CCHFV-GP38 13G8 (red) and mounted with mounting media containing a nuclear DAPI stain (blue). In the merged images, areas where there is overlapping expression of RABV-G and CCHFV-GP38 are pink. (FIG. 30C) Histograms and numerical values of flow cytometry staining of infected cells. Vero E6 cells were infected with either BNSP333-GP38 or BNSP333-GP85 at MOI 10 for 48 hrs and then fixed. Cells were then proved with α-RABV-G 4C12 and α-CCHFV-GP38 13G8 and analyzed by flow cytometry. Experiment was performed multiple times, and this is one representative experiment. (FIG. 30D) Western blot of sucrose purified virions. 1 μg of sucrose purified virions were run on an SDS PAGE gel and transferred to a nitrocellulose membrane for western blotting. Blots were probed with α-CCHFV-GP38 13G8. The image on the left is the merge of the visible and chemiluminescent channels to show the visible ladder markers, while the image on the right is just the chemiluminescent channel alone.

FIGS. 31A-31B: The adjuvant PHAD-SE boosts the antibody response of the vaccines, in accordance with some embodiments, α-CCHFV-GP38 total IgG ELISAs for sera from GP38+Gc−(FIG. 31A) and GP38+ Gc+ (FIG. 31B) immunized mice. Groups of 5 female mice were immunized with 10 μg per dose of BPL inactivated vaccine either with or without PHAD-SE adjuvant. ECso titers are compared over time between mice receiving unadjuvanted (solid symbols) and adjuvanted (clear symbols) vaccines. Error bars indicate the mean with standard deviation (SD) for groups of 5 mice with samples run in duplicate. The Mann-Whitney nonparametric t Test was used to determine statistical differences between groups at each time point. (**** P<0.0001; *** P<0.0002; ** P<0.0021; * P<0.0332; ns=not significant).

FIGS. 32A-32B: Pilot study of the surrogate challenge virus in IFNAR^{-/-} mice, in accordance with some embodiments. Groups of 5 male IFNAR″ mice were challenged I.P. with either 5e5, 7.5e5 or 1e6 pfu of the surrogate challenge virus (GP38+ Gc+). (FIG. 32A) Weight curves that represent the percent change in weight from the day of challenge. Dotted line represents 20% weight loss, the point at which mice were euthanized. Error bars indicate SD. (FIG. 32B) Levels RNA copies in the blood of mice as determined by qPCR for VSV-N. LOD, limit of detection. Error bars indicate the mean with SD.

FIG. 33A-33F: Individual group weight curves of mice challenged with the surrogate challenge virus, in accordance with some embodiments. Curves represent the percent change in weight from the day of challenge. Dotted line represents 20% weight loss, the point at which mice were euthanized. Results show the combination of two independent experiments; hollow symbols with a dotted connecting line represent the first experiment, and symbols with a black outline and solid connecting line represent the second experiment. Females from experiment two in panel A had their cage flooded on day 3, and thus the weights at this timepoint were excluded.

FIG. 34A-34B: Rhabdoviral-based CCHFV vaccines show no difference in immune responses between B6 males and females, in accordance with some embodiments. Total IgG ELISAs against GP38 (FIG. 34A) or Gc (FIG. 34B) with sera from mice immunized for the CCHFV WT challenge experiment. Groups of 10 mice, 5 male and 5 female, were immunized with 10 μg per dose of BPL inactivated vaccine adjuvanted with PHAD-SE. Error bars indicate the mean with standard deviation (SD) for groups of 5 mice with samples run in duplicate. An ordinary one-way ANOVA with Tukey's Multiple Comparison Test was used to determine statistical differences between groups at each time point. All groups with detectable antibody titers have 4-star significance compared to groups where no antibody titers were detected (**** P<0.0001; *** P<0.0002; ** P<0.0021; * P<0.0332; ns=not significant).

FIGS. 35A-35J: Clinical score heat maps from WT CCHFV challenge, in accordance with some embodiments. Mice were given a clinical score from 1-4 that is represented by colors in the bars next to the heat maps. Each row represents an individual mouse, labeled based on their group and ear notches. Criteria for scores are listed in Table 1 below the heat maps. Any time point where mice were not observed are crossed out with a gray X. The clinical scoring criteria is listed below:

TABLE 1
Clinical Scoring Criteria

1 Healthy
2 Ruffled fur, lethargic (triggers 2nd observation)
3 Ruffled fur, lethargic, hunched posture, orbital
tightening (Triggers 3rd observation)
4 Reluctance to move when stimulated, paralysis,
unable to access feed or water normally,
moribund appearance, or >20% weight loss
-- Immediate euthanasia --

### DETAILED DESCRIPTION

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. As used herein, nucleic acids include but are not limited to, all nucleic acid sequences which are obtained by any means available in the art, including, without limitation, recombinant means, i.e., the cloning of nucleic acid sequences from a recombinant library or a viral genome, using ordinary cloning technology and PCR™, and the like, and by synthetic means.

A “mutation” as used herein is a change in a DNA or amino acid sequence resulting in an alteration from its natural state. A mutation in a DNA sequence can comprise a deletion and/or insertion and/or duplication and/or substitution of at least one deoxyribonucleic acid base such as a purine (adenine and/or thymine) and/or a pyrimidine (guanine and/or cytosine) as compared to a reference sequence, e.g., a wildtype DNA sequence. A mutation in a protein or polypeptide sequence can comprise a deletion, insertion, or substitution of at least one amino acid residue, as compared to a reference sequence, e.g., a wildtype protein sequence.

As described herein, CCHFV refers to Crimean-Congo hemorrhagic fever virus. CCHFV is a member of the order Bunyavirales, a group of viruses that are single-stranded negative sense RNA viruses, generally with tri-segmented genomes. CCHFV is a member of the Orthonairovirus genus. CCHFV was first reported in the Crimean region as an acute hemorrhagic fever. Both, wild and domestic animals can serve as natural viral hosts. CCHFV have been associated with outbreaks of severe and fatal cases in Europe, Middle East, Asia and Africa. From 2002 to 2008, more than 2500 cases were reported only in Turkey. According to the WHO, CCHFV outbreaks have a fatality rate of up to 40%. Cases have also been associated with human-to-human transmission. A vaccine based on CCHFV, amplified in suckling mouse brain and inactivated by chloroform treatment, has been used in Eastern Europe, but is unlicensed by the European Medicines Agency or US Food and Drug Administration. A recent study found that it elicited both a cellular and humoral response to CCHFV, but neutralizing antibody titers were low, even in people who had received 4 doses. Controlled studies on protective efficacy have not been reported with this vaccine and, due to its crude preparation which raises concerns due to possible autoimmune and allergic responses induced by myelin basic protein; it is unlikely to gain widespread international regulatory approval. Several different vaccination approaches have been used for CCHFV such as inactivated virus vaccines, modified Vaccinia Ankara (MVA), Adenovirus-based vaccines, DNA vaccines, transgenic plant vaccines, recombinant protein based vaccines, virus like particles (VLP) based vaccines, but a vaccine that has demonstrated sufficient safety and efficacy in human use is still not available. Summarizing the above, to date, no safe and efficacious vaccine is available to protect against CCHFV infections.

As described herein, GP38 refers to glycoprotein 38. GP38 is part of the CCHFV medium (M) gene, which encodes for glycoproteins Gc, Gn and GP38. The composition of the GP38 protein varies among different CCHFV strains. In some embodiments, the nucleotide sequence encoding GP38 comprises the nucleotide sequence of SEQ ID NO: 2:

(SEQ ID NO: 2)
AACCTGAAGATGGAGATCATCCTGACCCTGAGCCAGGGCCTGAAGAAGT
ACTACGGCAAGATCCTGAGGCTGCTGCAGCTGACCCTGGAGGAGGACAC
CGAGGGCCTGCTGGAGTGGTGCAAGAGAAACCTGGGCCTGGACTGCGAC
GATACCTTCTTCCAGAAGCGGATCGAGGAGTTCTTCATCACCGGCGAGG
GCCACTTCAATGAAGTGCTGCAGTTCAGAACCCCCGGCACCCTGAGCAC
CACCGAGTCTACCCCTGCCGGCCTGCCCACCGCCGAGCCCTTCAAGAGC
TACTTCGCCAAGGGCTTCCTGAGCATCGACAGCGGCTACTACAGCGCCA
AGTGCTACAGCGGCACCTCCAACAGCGGACTGCAGCTGATCAACATCAC
CCGGCACAGCACCAGAATCGTGGATACCCCTGGCCCCAAGATCACCAAC
CTGAAAACCATCAACTGCATCAACCTGAAGGCCAGCATCTTCAAGGAGC
ACCGGGAAGTGGAGATCAACGTGCTGCTGCCCCAGGTGGCCGTGAATCT
GAGCAACTGCCACGTGGTGATCAAGAGCCATGTGTGCGACTACAGCCTG
GATATCGACGGCGCTGTGAGACTGCCCCACATCTACCACGAGGGCGTGT
TCATCCCTGGCACCTACAAGATCGTGATCGACAAGAAGAACAAGCTGAA
CGACCGGTGCACCCTGTTCACCGACTGCGTGATCAAGGGCCGGGAAGTG
AGAAAGGGCCAGAGCGTGCTGAGACAGTACAAGACCGAGATCCGGATCG
GCAAGGCCAGCACCGGGTCC

In some embodiments, the protein sequence of GP38 comprises the sequence of SEQ ID NO: 3:

(SEQ ID NO: 3)
NLKMEIILTLSQGLKKYYGKILRLLQLTLEEDTEGLLEWCKRNLGLDCD
DTFFQKRIEEFFITGEGHFNEVLQFRTPGTLSTTESTPAGLPTAEPFKS
YFAKGFLSIDSGYYSAKCYSGTSNSGLQLINITRHSTRIVDTPGPKITN
LKTINCINLKASIFKEHREVEINVLLPQVAVNLSNCHVVIKSHVCDYSL
DIDGAVRLPHIYHEGVFIPGTYKIVIDKKNKLNDRCTLFTDCVIKGREV
RKGQSVLRQYKTEIRIGKASTGS

In some embodiments, the viral glycoproteins interact with host cells to mediate viral entry, although the exact mechanisms of viral entry is unclear. CCHFV glycoproteins induce production of neutralizing and non-neutralizing antibodies in vivo. It has been shown that GP38 is required for eliciting protection in mice immunized with DNA vaccines containing the CCHFV glycoproteins.

As used herein, “MLD” refers to the “mucin-like domain” of CCHFV. The protein composition of the MLD varies among different CCHFV strains. In some embodiments, the MLD of CCHFV has the nucleotide sequence SEQ ID NO:4:

(SEQ ID NO: 4)
ATGCACATCAGCCTGATGTACGCCATCCTGTGCCTGCAGCTGTGCGGCC
TGGGCGAGACCCACGGCAGCCACAATGAGACCCGGCACAACAAGACCGA
CACCATGACCACCCCTGGCGACAACCCCAGCAGCGAGCCCCCTGTGAGC
ACCGCCCTGAGCATCACCCTGGATCCTAGCACCGTGACCCCCACCACCC
CTGCCAGCGGCCTGGAGGGCAGCGGCGAAGTGTACACCAGCCCCCCCAT
CACCACCGGCAGCCTGCCCCTGAGCGAGACCACCCCCGAGCTGCCCGTG
ACCACCGGCACCGATACCCTGAGCGCCGGAGATGTGGACCCCAGCACCC
AGACAGCCGGCGGAACCAGCGCCCCCACAGTGAGAACCAGCCTGCCCAA
TAGCCCTAGCACCCCAAGCACCCCTCAGGACACCCACCACCCTGTGAGA
AACCTGCTGAGCGTGACCAGCCCTGGCCCCGACGAGACCAGCACCCCCA
GCGGCACCGGCAAGGAGAGCAGCGCCACCTCCAGCCCCCACCCAGTGAG
CAATAGACCCCCTACCCCTCCCGCCACCGCCCAGGGCCCCACCGAGAAC
GACAGCCACAACGCCACCGAGCACCCCGAGAGCCTGACCCAGAGCGCCA
CCCCAGGCCTGATGACCAGCCCAACCCAGATCGTGCACCCCCAGTCCGC
CACCCCTATCACCGTGCAGGATACCCACCCCAGCCCCACCAACAGGAGC
AAGCGG

or the amino acid sequence SEQ ID NO:5:

(SEQ ID NO: 5)
MHISLMYAILCLQLCGLGETHGSHNETRHNKTDTMTTPGDNPSSEPPVS
TALSITLDPSTVTPTTPASGLEGSGEVYTSPPITTGSLPLSETTPELPV
TTGTDTLSAGDVDPSTQTAGGTSAPTVRTSLPNSPSTPSTPQDTHHPVR
NLLSVTSPGPDETSTPSGTGKESSATSSPHPVSNRPPTPPATAQGPTEN
DSHNATEHPESLTQSATPGLMTSPTQIVHPQSATPITVQDTHPSPTNRS
KR

As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

The term “antibody” or “Ab” as used herein, refers to a protein, or polypeptide sequence derived from an immunoglobulin molecule, which specifically binds to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F (ab) 2, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). An antibody may be derived from natural sources or from recombinant sources. Antibodies are typically tetramers of immunoglobulin molecules.

As used herein when referring to a measurable value such as an amount, a temporal duration, and the like, the term “about” is meant to encompass variations of +20% or +10%, more preferably +5%, even more preferably +1%, and still more preferably +0.1% from the specified value, as such variations are appropriate to perform the disclosed methods.

As used herein, the terms “biological sample” or “patient sample” or “test sample” or “sample” as used herein, refer to a sample obtained from an organism or from components (e.g., cells) of a subject or patient for the purpose of diagnosis, prognosis, or evaluation of a subject of interest. The sample can be, for example, blood which potentially is at risk of containing infection with CCHFV or rabies virus. In certain embodiments, such a sample may be obtained for assessing the presence of antibodies specific for CCHFV or a rabies virus following a suspected infection or following the vaccination using a vaccine construct of the invention. The invention contemplates the practice of any necessary safety and/or Governmental-imposed procedures for the handling and processing of any sample suspected of containing an infection with a rabies virus.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

As used herein, the terms “conditioning an immune response”, “eliciting an immune response” or “immunizing” refer to the process of generating a B cell and/or a T cell response against a heterologous protein. In some embodiments, parts of the heterologous protein function as an antigen.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, a “subject” includes human, nonhuman primate (e.g., ape or monkey), animal, e.g., horse, donkey, pig, mouse, hamster, monkey, chicken, and insect such as mosquito.

As used herein, the term “specifically binds to” or is “specific for” in the context of antibody/antigen interactions is intended to mean the specific binding of an antibody to a cognate antigen via specific one or more epitopes recognized by the antibody, without substantially binding to molecules that lack such epitopes.

As used herein, the term “treatment” or “treating” includes any process, action, application, therapy, or the like, wherein a subject (or patient), including a human being, is provided with or administered an agent or composition, e.g., a therapeutic vaccine composition, with the aim of improving the subject's condition, directly or indirectly, or slowing the progression of a condition or disorder in the subject (e.g., hemorrhagic fever or bleeding due to CCHFV infection), or ameliorating at least one symptom of the disease or disorder under treatment. As used in the context of disease caused by rabies, the terms “treat,” “treatment,” and the like, refer to relief from or alleviation of a pathological process mediated by said viruses.

The term “combination therapy” or “co-therapy” means the administration of two or more therapeutic agents to treat a disease, condition, and/or disorder, e.g., CCHFV-caused hemorrhagic fever. Such administration encompasses “co-administration” of two or more therapeutic agents in a substantially simultaneous manner. One therapy can be based on the dual-protective vaccines of the invention. A second therapy can be based on a known therapy for the disorder being treated. For example, alternative anti-virus drugs may be co-administered with the vaccine vectors of the invention. The order of administration of two or more sequentially co-administered therapeutic agents is not limited. The administration of the two or more therapeutic agents may also be administered by different routes, e.g., by a local route (e.g., mucosal delivery of a dual vaccine of the invention) and a systemic route (e.g., parenteral delivery of an anti-rabies small molecule inhibitor).

As used herein, the phrases “therapeutically effective amount” and “prophylactically effective amount” refer to an amount that provides a therapeutic benefit in the treatment, prevention, or management of pathological processes mediated by an infection with rabies virus, CCHFV or another bunyavirus, or an overt symptom of pathological processes mediated by rabies or CCHFV or another bunyavirus. The specific amount that is therapeutically effective can be readily determined by ordinary medical practitioner, and may vary depending on factors known in the art, such as, e.g. the type of pathological processes mediated by virus infection, the patient's history and age, the stage of pathological processes mediated by the virus infection, and the administration of other anti-pathological processes mediated by infection.

As used herein, a “vaccine construct” shall refer to a nucleic acid molecule constituting the recombinant rabies virus vector expressing one or more bunyavirus antigens (e.g., CCHFV GP38 glycoprotein) of the invention. The invention also contemplates the use of recombinant vaccine “virions” which are produced by the vaccine constructs of the invention when they are introduced into a host cell susceptible to infection therefrom, and which are then allowed to propagate and form whole virus virions in the cell, which are then obtained and/or purified. A “virion” refers to a complete virus particle resulting from an infection cycle of the recombinant rabies genome in a cell capable of hosting the rabies genome. The “vaccine” or “recombinant vaccines” of the invention encompass both “genetic vaccines,” i.e., the vaccine constructs of the invention, and the traditional vaccines, which are the virions themselves. Depending on the recombinant genome of the vaccine construct, the virions can be replication-competent or replication-deficient. Where they are replication-deficient, their propagation in host cells in vitro or in vivo may require a “helper” virus or cell, in which certain replication functions would be provided in trans by either the helper virus or the cell in which the infection is taking place. Vaccine compositions may also include both vaccine constructs as well as the virions themselves. The virions also may be of the “killed virus” type, whereby the virion is chemically treated or otherwise deactivated by some means of deactivation such that the virion has no or minimal ability to replication. Killed virus vaccines generally rely on their surface-presented polypeptides (e.g., the CCHFV GP38 protein) to induce a humoral-based immune response. Typically, a cellular-based immune response does not occur with the killed-virus type vaccines because these virions do not generally access the interior of cells.

As used herein, the term “isolated” or “purified” polypeptide or protein or virion or biologically-active portion or vaccine construct thereof is substantially free of cellular material or other contaminating proteins from the cell or tissue source from which the polypeptide (CCHFV GP38) is obtained.

As used herein, a “pharmaceutical composition” comprises a pharmacologically effective amount of a vaccine construct and a pharmaceutically acceptable carrier. As used herein, “pharmacologically effective amount,” “therapeutically effective amount” or simply “effective amount” refers to that amount of a vaccine effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter. Further, the pharmaceutical composition can be designed to enhance targeting cells involved in the underlying virus infection such as dendritic cells, macrophages, hepatocytes, and other parenchymal cells. As used herein, the term “pharmaceutically acceptable” means that the subject item is appropriate for use in a pharmaceutical product. The pharmaceutical composition facilitates administration of the compound to an organism. Multiple techniques of administering a compound exist in the art including, but not limited to: intravenous, oral, aerosol, parenteral, ophthalmic, pulmonary and topical administration.

As used herein, “pharmaceutically acceptable carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline or normal (0.9%) saline. Compositions comprising such carriers are formulated by well-known conventional methods (see, for example, Remington's Pharmaceutical Sciences, Chapter 43, 14th Ed., Mack Publishing Col, Easton Pa. 18042, USA). Pharmaceutically acceptable excipients have been amply described in a variety of publications, including, for example, A. Gennaro (2000) “Remington: The Science and Practice of Pharmacy,” 20th edition, Lippincott, Williams, & Wilkins; Remington's Pharmaceutical Sciences, 14th Ed. Or latest edition, Mack Publishing Col, Easton Pa. 18042, USA; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. Further discussion is provided herein.

As used herein, an “adjuvant” is a component added to a pharmaceutical composition. In various embodiments, the adjuvant may be any substance that acts to accelerate, prolong, or enhance antigen-specific immune response when used in combination with the pharmaceutical composition. In various embodiments, the adjuvant is an inorganic compound, an organic compound, an oil, a plant derived compound, or a cytokine. In various embodiments, the adjuvant is an aluminum salt. In various embodiments the adjuvant is potassium alum, aluminum hydroxide, aluminum phosphate. In various embodiments, the adjuvant is squalene. In various embodiments, the adjuvant is a synthetic toll-like receptor 4 agonist (Monophosphoryl hexa-acyl lipid A, 3-deacyl (PHAD)) in a stable oil-in-water emulsion (SE). In various embodiments the adjuvant is paraffin oil. In various embodiments the adjuvant is a saponin. In various embodiments the adjuvant is IL-1, IL-2 or IL 12. In various embodiments, the adjuvant is Freunds complete adjuvant. In various embodiments the adjuvant is a toxoid.

As used herein, the term “effective amount” or “therapeutically effective amount” means the amount of the virus like particle generated from vector of the invention which is required to prevent the particular disease condition, or which reduces the severity of and/or ameliorates the disease condition or at least one symptom thereof or condition associated therewith.

“Titers” are numerical measures of the concentration of a virus or viral vector compared to a reference sample, where the concentration is determined either by the activity of the virus, or by measuring the number of viruses in a unit volume of buffer. The titer of viral stocks are determined, e.g., by measuring the infectivity of a solution or solutions (typically serial dilutions) of the viruses, e.g., a focus-forming assay adapted from established methods (Pulmanausahakul, Li, Schnell & Dietzschold (2008) J Virol 82 (5): 2330 LP-2338) or by a well-established plaque assay using methylcellulose (Burleson, Chambers & Wiedbrauk (1992) Virology 16:74-84). on HeLa cells using the soft agar method (see, Graham & Van Der eb (1973) Virology 52:456-467) or by monitoring resistance conferred to cells, e.g., G418 resistance encoded by the virus or vector, or by quantitating the viruses by UV spectrophotometry (see, Chardonnet & Dales (1970) Virology 40:462-477).

“Vaccination” refers to the process of inoculating a subject with an antigen to elicit an immune response in the subject, that helps to prevent or treat the disease or disorder the antigen is connected with. The term “immunization” is used interchangeably herein with vaccination.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In the present disclosure, the term “vector” includes an autonomously replicating virus.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention contemplates that any suitable rabies virus genome or vector can be used to construct the recombinant vaccines of the invention. Thus, the rabies virus genome can be obtained from any suitable strain or isolate of rabies virus, so long as it is or is made to be attenuated. For the purposes of this invention, the term “attenuated,” as it pertains to a property of a rabies virus genome of the invention, shall mean that the rabies virus genome or vector is capable of viral attachment, entry, and in some cases, replication in a host cell. However, attenuated rabies virus genomes—as compared to non-attenuated rabies viruses or rabies virus genomes—have substantially or completely lost the property of neurovirulence. In other words, the neurotropic character of the attenuated RVs of the invention preferably have been abolished or substantially abolished such that the RV vectors of the invention are safe for administering to a subject or animal without a substantial concern for neurovirulence effects.

The basic biology of the rabies virus is well-known. Rabies virus is a non-segmented negative-strand RNA virus of the rhabdoviridae family, and which is the causative agent of rabies. Rabies is a disease that can occur in all warm-blooded species. Infection with rabies virus followed by the outbreak of the clinical features in nearly all instances results in death of the infected species. In Europe, the USA and Canada wildlife rabies still exists and is an important factor in the cause of most human rabies cases that occur. On the other hand, urban rabies constitutes the major cause of human rabies in developing countries and entire continents, such as Africa.

Rabies virus (RV) virions are composed of two major structural components: a nucleocapsid or ribonucleoprotein (RNP), and an envelope in the form of a bilayer membrane surrounding the RNP core. The infectious component of all Rhabdoviruses is the RNP core which consists of the RNA genome encapsidated by the nucleocapsid (N) protein in combination with two minor proteins, i.e. RNA-dependent RNA-polymerase (L) and phosphoprotein (P). The membrane surrounding the RNP core consists of two proteins: a trans-membrane glycoprotein (G) and a matrix (M) protein located at the inner site of the membrane.

The G protein, also referred to as spike protein, is responsible for cell attachment and membrane fusion in RV and additionally is the main target for the host immune system. The amino acid region at position 330 to 340 (referred to as antigenic site III) of the G protein has been identified to be responsible for the virulence of the virus, in particular the Arg residue at position 333. All RV strains have this virulence determining antigenic site III in common.

Although wildtype rabies virus almost always causes a fatal central nervous system (CNS) disease in mammalian species, attenuated form(s) of rabies virus typically do not cause such problems.

Suitable attenuated rabies virus genome or vectors can be found described elsewhere, for example, in U.S. Pat. Nos. 7,544,791; 7,419,816; 6,887,479; 6,719,981; and 6,706,523, each of which are incorporated herein by reference.

In a preferred embodiment, the attenuated rabies virus genome of the invention is based on the replication-competent rabies virus strain SAD B19, which is a RV strain that has been used for oral immunization of wild-life animals in Europe for more than 20 years and which has a good safety record. The nucleotide sequence for SAD B19 is publicly available as Genbank accession No. M31046.1.

### Compositions

In one aspect, the invention provides a composition comprising a recombinant vector from an attenuated rabies virus comprising a nucleotide sequence encoding at least one CCHFV glycoprotein and a mucin-like domain.

In some embodiments, the at least one CCHFV glycoprotein is GP38, or an antigenic fragment thereof.

In some embodiments, a recombinant rabies vector was designed expressing a chimeric CCHFV GP38 (BNSP333-GP85), which has a human Ig-kappa signal sequence for ER translocation, the mucin-like domain of CCHFV, and 51 amino acids of the ectodomain, transmembrane domain and cytoplasmic domain of RABV G (ED51). The mucin-like domain was included in the construct because it is necessary for proper processing of GP38, however, it will not be incorporated into the virion because it is cleaved from GP38. In some embodiments, GP38 is cleaved off MLD by cellular proteases.

In some embodiments, the nucleotide sequence encoding the recombinant rabies vector has at least 80% sequence identity with SEQ ID NO: 1. In some embodiments, the nucleotide sequence encoding the recombinant rabies vector has at least 85%, at least 90%, at least 95%, at least 98%, at least 99% or 100% sequence identity with SEQ ID NO: 1. In various embodiments, the recombinant rabies virus vector comprising a nucleotide sequence encoding at least one CCHFV glycoprotein and at least one mucin-like domain (MLD), comprises the nucleotide sequence of SEQ ID NO:1:

(SEQ ID NO: 1)
CTGACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTG
ATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTA
TCTCGGTCTATTCTTTTGATTTATAAGGGATTTTGCCGATTTCGGCCTATTGGTTAAA
AAATGAGCTGATTTAACAAAAATTTAACGCGAATTTTAACAAAATATTAACGCTTAC
AATTTCCATTCGCCATTCAGGCTGCGCAACTGTTGGGAAGGGCGATCGGTGCGGGCC
TCTTCGCTATTACGCCAGCTGGCGAAAGGGGGATGTGCTGCAAGGCGATTAAGTTGG
GTAACGCCAGGGTTTTCCCAGTCACGACGTTGTAAAACGACGGCCAGTGAGcgcgccC
TAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTC
TCTGGCTAACTAGAGAACCCACTGCTTACTGGCTTATCGAAATTAATACGACTCACT
ATAGGGAGACCCAAGCTGGCTAGATTAAGCGTCTGATGAGTCCGTGAGGACGAAAC
CCGGCGTACCGGGTCACGCTTAACAACCAGATCAAAGAAAAAACAGACATTGTCAA
TTGCAAAGCAAAAATGTAACACCCCTACAATGGATGCCGACAAGATTGTATTCAAA
GTCAATAATCAGGTGGTCTCTTTGAAGCCTGAGATTATCGTGGATCAATATGAGTAC
AAGTACCCTGCCATCAAAGATTTGAAAAAGCCCTGTATAACCCTAGGAAAGGCTCC
CGATTTAAATAAAGCATACAAGTCAGTTTTGTCAGGCATGAGCGCCGCCAAACTTAA
TCCTGACGATGTATGTTCCTATTTGGCAGCGGCAATGCAGTTTTTTGAGGGGACATG
TCCGGAAGACTGGACCAGCTATGGAATTGTGATTGCACGAAAAGGAGATAAGATCA
CCCCAGGTTCTCTGGTGGAGATAAAACGTACTGATGTAGAAGGGAATTGGGCTCTG
ACAGGAGGCATGGAACTGACAAGAGACCCCACTGTCCCTGAGCATGCGTCCTTAGT
CGGTCTTCTCTTGAGTCTGTATAGGTTGAGCAAAATATCCGGGCAAAACACTGGTAA
CTATAAGACAAACATTGCAGACAGGATAGAGCAGATTTTTGAGACAGCCCCTTTTGT
TAAAATCGTGGAACACCATACTCTAATGACAACTCACAAaATGTGTGCTAATTGGAG
TACTATACCAAACTTCAGATTTTTGGCCGGAACCTATGACATGTTTTTCTCCCGGATT
GAGCATCTATATTCAGCAATCAGAGTGGGCACAGTTGTCACTGCTTATGAAGACTGT
TCAGGACTGGTATCATTTACTGGGTTCATAAAACAAATCAATCTCACCGCTAGAGAG
GCAATACTATATTTCTTCCACAAGAACTTTGAGGAAGAGATAAGAAGAATGTTTGAG
CCAGGGCAGGAGACAGCTGTTCCTCACTCTTATTTCATCCACTTCCGTTCACTAGGCT
TGAGTGGGAAATCTCCTTATTCATCAAATGCTGTTGGTCACGTGTTCAATCTCATTCA
CTTTGTAGGATGCTATATGGGTCAAGTCAGATCCCTAAATGCAACGGTTATTGCTGC
ATGTGCTCCTCATGAAATGTCTGTTCTAGGGGGCTATCTGGGAGAGGAATTCTTCGG
GAAAGGGACATTTGAAAGAAGATTCTTCAGAGATGAGAAAGAACTTCAAGAATACG
AGGCGGCTGAACTGACAAAGACTGACGTAGCACTGGCAGATGATGGAACTGTCAAC
TCTGACGACGAGGACTACTTTTCAGGTGAAACCAGAAGTCCGGAGGCTGTTTATACT
CGAATCATGATGAATGGAGGTCGACTAAAGAGATCTCACATACGGAGATATGTCTC
AGTCAGTTCCAATCATCAAGCCCGTCCAAACTCATTCGCCGAGTTTCTAAACAAGAC
ATATTCGAGTGACTCATAACATGAAAAAAACTAACACCCCTCCCGTACGGCCACCAT
GCACATCAGCCTGATGTACGCCATCCTGTGCCTGCAGCTGTGCGGCCTGGGCGAGAC
CCACGGCAGCCACAATGAGACCCGGCACAACAAGACCGACACCATGACCACCCCTG
GCGACAACCCCAGCAGCGAGCCCCCTGTGAGCACCGCCCTGAGCATCACCCTGGAT
CCTAGCACCGTGACCCCCACCACCCCTGCCAGCGGCCTGGAGGGCAGCGGCGAAGT
GTACACCAGCCCCCCCATCACCACCGGCAGCCTGCCCCTGAGCGAGACCACCCCCG
AGCTGCCCGTGACCACCGGCACCGATACCCTGAGCGCCGGAGATGTGGACCCCAGC
ACCCAGACAGCCGGCGGAACCAGCGCCCCCACAGTGAGAACCAGCCTGCCCAATAG
CCCTAGCACCCCAAGCACCCCTCAGGACACCCACCACCCTGTGAGAAACCTGCTGA
GCGTGACCAGCCCTGGCCCCGACGAGACCAGCACCCCCAGCGGCACCGGCAAGGAG
AGCAGCGCCACCTCCAGCCCCCACCCAGTGAGCAATAGACCCCCTACCCCTCCCGCC
ACCGCCCAGGGCCCCACCGAGAACGACAGCCACAACGCCACCGAGCACCCCGAGA
GCCTGACCCAGAGCGCCACCCCAGGCCTGATGACCAGCCCAACCCAGATCGTGCAC
CCCCAGTCCGCCACCCCTATCACCGTGCAGGATACCCACCCCAGCCCCACCAACAGG
AGCAAGCGGAACCTGAAGATGGAGATCATCCTGACCCTGAGCCAGGGCCTGAAGAA
GTACTACGGCAAGATCCTGAGGCTGCTGCAGCTGACCCTGGAGGAGGACACCGAGG
GCCTGCTGGAGTGGTGCAAGAGAAACCTGGGCCTGGACTGCGACGATACCTTCTTCC
AGAAGCGGATCGAGGAGTTCTTCATCACCGGCGAGGGCCACTTCAATGAAGTGCTG
CAGTTCAGAACCCCCGGCACCCTGAGCACCACCGAGTCTACCCCTGCCGGCCTGCCC
ACCGCCGAGCCCTTCAAGAGCTACTTCGCCAAGGGCTTCCTGAGCATCGACAGCGG
CTACTACAGCGCCAAGTGCTACAGCGGCACCTCCAACAGCGGACTGCAGCTGATCA
ACATCACCCGGCACAGCACCAGAATCGTGGATACCCCTGGCCCCAAGATCACCAAC
CTGAAAACCATCAACTGCATCAACCTGAAGGCCAGCATCTTCAAGGAGCACCGGGA
AGTGGAGATCAACGTGCTGCTGCCCCAGGTGGCCGTGAATCTGAGCAACTGCCACG
TGGTGATCAAGAGCCATGTGTGCGACTACAGCCTGGATATCGACGGCGCTGTGAGA
CTGCCCCACATCTACCACGAGGGCGTGTTCATCCCTGGCACCTACAAGATCGTGATC
GACAAGAAGAACAAGCTGAACGACCGGTGCACCCTGTTCACCGACTGCGTGATCAA
GGGCCGGGAAGTGAGAAAGGGCCAGAGCGTGCTGAGACAGTACAAGACCGAGATC
CGGATCGGCAAGGCCAGCACCGGGTCCGAATCCTCGGTTATCCCCCTTGTGCACCCC
CTGGCAGACCCGTCTACCGTTTTCAAGGACGGTGACGAGGCTGAGGATTTTGTTGAA
GTTCACCTTCCCGATGTGCACAATCAGGTCTCAGGAGTTGACTTGGGTCTCCCGAAC
TGGGGGAAGTATGTATTACTGAGTGCAGGGGCCCTGACTGCCTTGATGTTGATAATT
TTCCTGATGACATGTTGTAGAAGAGTCAATCGATCAGAACCTACGCAACACAATCTC
AGAGGGACAGGGAGGGAGGTGTCAGTCACTCCCCAAAGCGGGAAGATCATATCTTC
ATGGGAATCACACAAGAGTGGGGGTGAGACCAGACTGTAAGCTAGCCATGAAAAA
AACTAACACCCCTCCTTTCGAACCATCCCAAACATGAGCAAGATCTTTGTCAATCCT
AGTGCTATTAGAGCCGGTCTGGCCGATCTTGAGATGGCTGAAGAAACTGTTGATCTG
ATCAATAGAAATATCGAAGACAATCAGGCTCATCTCCAAGGGGAACCCATAGAGGT
GGACAATCTCCCTGAGGATATGGGGCGACTTCACCTGGATGATGGAAAATCGCCCA
ACCATGGTGAGATAGCCAAGGTGGGAGAAGGCAAGTATCGAGAGGACTTTCAGATG
GATGAAGGAGAGGATCCTAGCTTCCTGTTCCAGTCATACCTGGAAAATGTTGGAGTC
CAAATAGTCAGACAAATGAGGTCAGGAGAGAGATTTCTCAAGATATGGTCACAGAC
CGTAGAAGAGATTATATCCTATGTCGCGGTCAACTTTCCCAACCCTCCAGGAAAGTC
TTCAGAGGATAAATCAACCCAGACTACTGGCCGAGAGCTCAAGAAGGAGACAACAC
CCACTCCTTCTCAGAGAGAAAGCCAATCATCGAAAGCCAGGATGGCGGCTCAAATT
GCTTCTGGCCCTCCAGCCCTTGAATGGTCGGCTACCAATGAAGAGGATGATCTATCA
GTGGAGGCTGAGATCGCTCACCAGATTGCAGAAAGTTTCTCCAAAAAATATAAGTTT
CCCTCTCGATCCTCAGGGATACTCTTGTATAATTTTGAGCAATTGAAAATGAACCTT
GATGATATAGTTAAAGAGGCAAAAAATGTACCAGGTGTGACCCGTTTAGCCCATGA
CGGGTCCAAACTCCCCCTAAGATGTGTACTGGGATGGGTCGCTTTGGCCAACTCTAA
GAAATTCCAGTTGTTAGTCGAATCCGACAAGCTGAGTAAAATCATGCAAGATGACTT
GAATCGCTATACATCTTGCTAACCGAACCTCTCCCCTCAGTCCCTCTAGACAATAAA
ATCCGAGATGTCCCAAAGTCAACATGAAAAAAACAGGCAACACCACTGATAAAATG
AACCTCCTACGTAAGATAGTGAAAAACCGCAGGGACGAGGACACTCAAAAATCCTC
TCCCGCGTCAGCCCCTCTGGATGACGATGACTTGTGGCTTCCACCCCCTGAATACGT
CCCGCTGAAAGAACTTACAGGCAAGAAGAACATGAGGAACTTTTGTATCAACGGAA
GGGTTAAAGTGTGTAGCCCGAATGGTTACTCGTTCAGGATCCTGCGGCACATTCTGA
AATCATTCGACGAGATATATTCTGGGAATCATAGGATGATCGGGTTAGTCAAAGTGG
TTATTGGACTGGCTTTGTCAGGATCTCCAGTCCCTGAGGGCCTGAACTGGGTATACA
AATTGAGGAGAACCTTTATCTTCCAGTGGGCTGATTCCAGGGGCCCTCTTGAAGGGG
AGGAGTTGGAATACTCTCAGGAGATCACTTGGGATGATGATACTGAGTTCGTCGGAT
TGCAAATAAGAGTGATTGCAAAACAGTGTCATATCCAGGGCAGAGTCTGGTGTATC
AACATGAACCCGAGAGCATGTCAACTATGGTCTGACATGTCTCTTCAGACACAAAG
GTCCGAAGAGGACAAAGATTCCTCTCTGCTTCTAGAATAATCAGATTATATCCCGCA
AATTTATCACTTGTTTACCTCTGGAGGAGAGAACATATGGGCTCAACTCCAACCCTT
GGGAGCAATATAACAAAAAACATGTTATGGTGCCATTAAACCGCTGCATTTCATCAA
AGTCAAGTTGATTACCTTTACATTTTGATCCTCTTGGATGTGAAAAAAACTATTAAC
ATCCCTCAAAAGACCCCGGGAAAGATGGTTCCTCAGGCTCTCCTGTTTGTACCCCTT
CTGGTTTTTCCATTGTGTTTTGGGAAATTCCCTATTTACACGATACCAGACAAGCTTG
GTCCCTGGAGTCCGATTGACATACATCACCTCAGCTGCCCAAACAATTTGGTAGTGG
AGGACGAAGGATGCACCAACCTGTCAGGGTTCTCCTACATGGAACTTAAAGTTGGA
TACATCTTAGCCATAAAAGTGAACGGGTTCACTTGCACAGGCGTTGTGACGGAGGCT
GAAACCTACACTAACTTCGTTGGTTATGTCACAACCACGTTCAAAAGAAAGCATTTC
CGCCCAACACCAGATGCATGTAGAGCCGCGTACAACTGGAAGATGGCCGGTGACCC
CAGATATGAAGAGTCTCTACACAATCCGTACCCTGACTACCGCTGGCTTCGAACTGT
AAAAACCACCAAGGAGTCTCTCGTTATCATATCTCCAAGTGTGGCAGATTTGGACCC
ATATGACAGATCCCTTCACTCGAGGGTCTTCCCTAGCGGGAAGTGCTCAGGAGTAGC
GGTGTCTTCTACCTACTGCTCCACTAACCACGATTACACCATTTGGATGCCCGAGAA
TCCGAGACTAGGGATGTCTTGTGACATTTTTACCAATAGTAGAGGGAAGAGAGCATC
CAAAGGGAGTGAGACTTGCGGCTTTGTAGATGAAAGAGGCCTATATAAGTCTTTAA
AAGGAGCATGCAAACTCAAGTTATGTGGAGTTCTAGGACTTAGACTTATGGATGGA
ACATGGGTCTCGATGCAAACATCAAATGAAACCAAATGGTGCCCTCCCGATAAGTT
GGTGAACCTGCACGACTTTCGCTCAGACGAAATTGAGCACCTTGTTGTAGAGGAGTT
GGTCAGGAAGAGAGAGGAGTGTCTGGATGCACTAGAGTCCATCATGACAACCAAGT
CAGTGAGTTTCAGACGTCTCAGTCATTTAAGAAAACTTGTCCCTGGGTTTGGAAAAG
CATATACCATATTCAACAAGACCTTGATGGAAGCCGATGCTCACTACAAGTCAGTCg
agACTTGGAATGAGATCCTCCCTTCAAAAGGGTGTTTAAGAGTTGGGGGGAGGTGTC
ATCCTCATGTGAACGGGGTGTTTTTCAATGGTATAATATTAGGACCTGACGGCAATG
TCTTAATCCCAGAGATGCAATCATCCCTCCTCCAGCAACATATGGAGTTGTTGGAAT
CCTCGGTTATCCCCCTTGTGCACCCCCTGGCAGACCCGTCTACCGTTTTCAAGGACG
GTGACGAGGCTGAGGATTTTGTTGAAGTTCACCTTCCCGATGTGCACAATCAGGTCT
CAGGAGTTGACTTGGGTCTCCCGAACTGGGGGAAGTATGTATTACTGAGTGCAGGG
GCCCTGACTGCCTTGATGTTGATAATTTTCCTGATGACATGTTGTAGAAGAGTCAAT
CGATCAGAACCTACGCAACACAATCTCAGAGGGACAGGGAGGGAGGTGTCAGTCAC
TCCCCAAAGCGGGAAGATCATATCTTCATGGGAATCACACAAGAGTGGGGGTGAGA
CCAGACTGTAATTAATTAACGTCCTTTCAACGATCCAAGTCCATGAAAAAAACTAAC
ACCCCTCCCGTACCTAGCTTATAAAGTGCTGGGTCATCTAAGCTTTTCAGTCGAGAA
AAAAACATTAGATCAGAAGAACAACTGGCAACACTTCTCAACCTGAGACTTACTTC
AAGATGCTCGATCCTGGAGAGGTCTATGATGACCCTATTGACCCAATCGAGTTAGAG
GCTGAACCCAGAGGAACCCCCATTGTCCCCAACATCTTGAGGAACTCTGACTACAAT
CTCAACTCTCCTTTGATAGAAGATCCTGCTAGACTAATGTTAGAATGGTTAAAAACA
GGGAATAGACCTTATCGGATGACTCTAACAGACAATTGCTCCAGGTCTTTCAGAGTT
TTGAAAGATTATTTCAAGAAGGTAGATTTGGGTTCTCTCAAGGTGGGCGGAATGGCT
GCACAGTCAATGATTTCTCTCTGGTTATATGGTGCCCACTCTGAATCCAACAGGAGC
CGGAGATGTATAACAGACTTGGCCCATTTCTATTCCAAGTCGTCCCCCATAGAGAAG
CTGTTGAATCTCACGCTAGGAAATAGAGGGCTGAGAATCCCCCCAGAGGGAGTGTT
AAGTTGCCTTGAGAGGGTTGATTATGATAATGCATTTGGAAGGTATCTTGCCAACAC
GTATTCCTCTTACTTGTTCTTCCATGTAATCACCTTATACATGAACGCCCTAGACTGG
GATGAAGAAAAGACCATCCTAGCATTATGGAAAGATTTAACCTCAGTGGACATCGG
GAAGGACTTGGTAAAGTTCAAAGACCAAATATGGGGACTGCTGATCGTGACAAAGG
ACTTTGTTTACTCCCAAAGTTCCAATTGTCTTTTTGACAGAAACTACACACTTATGCT
AAAAGATCTTTTCTTGTCTCGCTTCAACTCCTTAATGGTCTTGCTCTCTCCCCCAGAG
CCCCGATACTCAGATGACTTGATATCTCAACTATGCCAGCTGTACATTGCTGGGGAT
CAAGTCTTGTCTATGTGTGGAAACTCCGGCTATGAAGTCATCAAAATATTGGAGCCA
TATGTCGTGAATAGTTTAGTCCAGAGAGCAGAAAAGTTTAGGCCTCTCATTCATTCC
TTGGGAGACTTTCCTGTATTTATAAAAGACAAGGTAAGTCAACTTGAAGAGACGTTC
GGTCCCTGTGCAAGAAGGTTCTTTAGGGCTCTGGATCAATTCGACAACATACATGAC
TTGGTTTTTGTGTTTGGCTGTTACAGGCATTGGGGGCACCCATATATAGATTATCGAA
AGGGTCTGTCAAAACTATATGATCAGGTTCACCTTAAAAAAATGATAGATAAGTCCT
ACCAGGAGTGCTTAGCAAGCGACCTAGCCAGGAGGATCCTTAGATGGGGTTTTGAT
AAGTACTCCAAGTGGTATCTGGATTCAAGATTCCTAGCCCGAGACCACCCCTTGACT
CCTTATATCAAAACCCAAACATGGCCACCCAAACATATTGTAGACTTGGTGGGGGAT
ACATGGCACAAGCTCCCGATCACGCAGATCTTTGAGATTCCTGAATCAATGGATCCG
TCAGAAATATTGGATGACAAATCACATTCTTTCACCAGAACGAGACTAGCTTCTTGG
CTGTCAGAAAACCGAGGGGGGCCTGTTCCTAGCGAAAAAGTTATTATCACGGCCCT
GTCTAAGCCGCCTGTCAATCCCCGAGAGTTTCTGAGGTCTATAGACCTCGGAGGATT
GCCAGATGAAGACTTGATAATTGGCCTCAAGCCAAAGGAACGGGAATTGAAGATTG
AAGGTCGATTCTTTGCTCTAATGTCATGGAATCTAAGATTGTATTTTGTCATCACTGA
AAAACTCTTGGCCAACTACATCTTGCCACTTTTTGACGCGCTGACTATGACAGACAA
CCTGAACAAGGTGTTTAAAAAGCTGATCGACAGGGTCACCGGGCAAGGGCTTTTGG
ACTATTCAAGGGTCACATATGCATTTCACCTGGACTATGAAAAGTGGAACAACCATC
AAAGATTAGAGTCAACAGAGGATGTATTTTCTGTCCTAGATCAAGTGTTTGGATTGA
AGAGAGTGTTTTCTAGAACACACGAGTTTTTTCAAAAGGCCTGGATCTATTATTCAG
ACAGATCAGACCTCATCGGGTTACGGGAGGATCAAATATACTGCTTAGATGCGTCCA
ACGGCCCAACCTGTTGGAATGGCCAGGATGGCGGGCTAGAAGGCTTACGGCAGAAG
GGCTGGAGTCTAGTCAGCTTATTGATGATAGATAGAGAATCTCAAATCAGGAACAC
AAGAACCAAAATACTAGCTCAAGGAGACAACCAGGTTTTATGTCCGACATACATGT
TGTCGCCAGGGCTATCTCAAGAGGGGCTCCTCTATGAATTGGAGAGAATATCAAGG
AATGCACTTTCGATATACAGAGCCGTCGAGGAAGGGGCATCTAAGCTAGGGCTGAT
CATCAAGAAAGAAGAGACCATGTGTAGTTATGACTTCCTCATCTATGGAAAAACCCC
TTTGTTTAGAGGTAACATATTGGTGCCTGAGTCCAAAAGATGGGCCAGAGTCTCTTG
CGTCTCTAATGACCAAATAGTCAACCTCGCCAATATAATGTCGACAGTGTCCACCAA
TGCGCTAACAGTGGCACAACACTCTCAATCTTTGATCAAACCGATGAGGGATTTTCT
GCTCATGTCAGTACAGGCAGTCTTTCACTACCTGCTATTTAGCCCAATCTTAAAGGG
AAGAGTTTACAAGATTCTGAGCGCTGAAGGGGAGAGCTTTCTCCTAGCCATGTCAAG
GATAATCTATCTAGATCCTTCTTTGGGAGGGATATCTGGAATGTCCCTCGGAAGATT
CCATATACGACAGTTCTCAGACCCTGTCTCTGAAGGGTTATCCTTCTGGAGAGAGAT
CTGGTTAAGCTCCCAAGAGTCCTGGATTCACGCGTTGTGTCAAGAGGCTGGAAACCC
AGATCTTGGAGAGAGAACACTCGAGAGCTTCACTCGCCTTCTAGAAGATCCGACCA
CCTTAAATATCAGAGGAGGGGCCAGTCCTACCATTCTACTCAAGGATGCAATCAGA
AAGGCTTTATATGACGAGGTGGACAAGGTGGAAAATTCAGAGTTTCGAGAGGCAAT
CCTGTTGTCCAAGACCCATAGAGATAATTTTATACTCTTCTTAATATCTGTTGAGCCT
CTGTTTCCTCGATTTCTCAGTGAGCTATTCAGTTCGTCTTTTTTGGGAATCCCCGAGT
CAATCATTGGATTGATACAAAACTCCCGAACGATAAGAAGGCAGTTTAGAAAGAGT
CTCTCAAAAACTTTAGAAGAATCCTTCTACAACTCAGAGATCCACGGGATTAGTCGG
ATGACCCAGACACCTCAGAGGGTTGGGGGGGTGTGGCCTTGCTCTTCAGAGAGGGC
AGATCTACTTAGGGAGATCTCTTGGGGAAGAAAAGTGGTAGGCACGACAGTTCCTC
ACCCTTCTGAGATGTTGGGATTACTTCCCAAGTCCTCTATTTCTTGCACTTGTGGAGC
AACAGGAGGAGGCAATCCTAGAGTTTCTGTATCAGTACTCCCGTCCTTTGATCAGTC
ATTTTTTTCACGAGGCCCCCTAAAGGGATACTTGGGCTCGTCCACCTCTATGTCGACC
CAGCTATTCCATGCATGGGAAAAAGTCACTAATGTTCATGTGGTGAAGAGAGCTCTA
TCGTTAAAAGAATCTATAAACTGGTTCATTACTAGAGATTCCAACTTGGCTCAAGCT
CTAATTAGGAACATTATGTCTCTGACAGGCCCTGATTTCCCTCTAGAGGAGGCCCCT
GTCTTCAAAAGGACGGGGTCAGCCTTGCATAGGTTCAAGTCTGCCAGATACAGCGA
AGGAGGGTATTCTTCTGTCTGCCCGAACCTCCTCTCTCATATTTCTGTTAGTACAGAC
ACCATGTCTGATTTGACCCAAGACGGGAAGAACTACGATTTCATGTTCCAGCCATTG
ATGCTTTATGCACAGACATGGACATCAGAGCTGGTACAGAGAGACACAAGGCTAAG
AGACTCTACGTTTCATTGGCACCTCCGATGCAACAGGTGTGTGAGACCCATTGACGA
CGTGACCCTGGAGACCTCTCAGATCTTCGAGTTTCCGGATGTGTCGAAAAGAATATC
CAGAATGGTTTCTGGGGCTGTGCCTCACTTCCAGAGGCTTCCCGATATCCGTCTGAG
ACCAGGAGATTTTGAATCTCTAAGCGGTAGAGAAAAGTCTCACCATATCGGATCAG
CTCAGGGGCTCTTATACTCAATCTTAGTGGCAATTCACGACTCAGGATACAATGATG
GAACCATCTTCCCTGTCAACATATACGGCAAGGTTTCCCCTAGAGACTATTTGAGAG
GGCTCGCAAGGGGAGTATTGATAGGATCCTCGATTTGCTTCTTGACAAGAATGACAA
ATATCAATATTAATAGACCTCTTGAATTGGTCTCAGGGGTAATCTCATATATTCTCCT
GAGGCTAGATAACCATCCCTCCTTGTACATAATGCTCAGAGAACCGTCTCTTAGAGG
AGAGATATTTTCTATCCCTCAGAAAATCCCCGCCGCTTATCCAACCACTATGAAAGA
AGGCAACAGATCAATCTTGTGTTATCTCCAACATGTGCTACGCTATGAGCGAGAGAT
AATCACGGCGTCTCCAGAGAATGACTGGCTATGGATCTTTTCAGACTTTAGAAGTGC
CAAAATGACGTACCTATCCCTCATTACTTACCAGTCTCATCTTCTACTCCAGAGGGTT
GAGAGAAACCTATCTAAGAGTATGAGAGATAACCTGCGACAATTGAGTTCTTTGAT
GAGGCAGGTGCTGGGCGGGCACGGAGAAGATACCTTAGAGTCAGACGACAACATTC
AACGACTGCTAAAAGACTCTTTACGAAGGACAAGATGGGTGGATCAAGAGGTGCGC
CATGCAGCTAGAACCATGACTGGAGATTACAGCCCCAACAAGAAGGTGTCCCGTAA
GGTAGGATGTTCAGAATGGGTCTGCTCTGCTCAACAGGTTGCAGTCTCTACCTCAGC
AAACCCGGCCCCTGTCTCGGAGCTTGACATAAGGGCCCTCTCTAAGAGGTTCCAGAA
CCCTTTGATCTCGGGCTTGAGAGTGGTTCAGTGGGCAACCGGTGCTCATTATAAGCT
TAAGCCTATTCTAGATGATCTCAATGTTTTCCCATCTCTCTGCCTTGTAGTTGGGGAC
GGGTCAGGGGGGATATCAAGGGCAGTCCTCAACATGTTTCCAGATGCCAAGCTTGT
GTTCAACAGTCTTTTAGAGGTGAATGACCTGATGGCTTCCGGAACACATCCACTGCC
TCCTTCAGCAATCATGAGGGGAGGAAATGATATCGTCTCCAGAGTGATAGATCTTGA
CTCAATCTGGGAAAAACCGTCCGACTTGAGAAACTTGGCAACCTGGAAATACTTCCA
GTCAGTCCAAAAGCAGGTCAACATGTCCTATGACCTCATTATTTGCGATGCAGAAGT
TACTGACATTGCATCTATCAACCGGATCACCCTGTTAATGTCCGATTTTGCATTGTCT
ATAGATGGACCACTCTATTTGGTCTTCAAAACTTATGGGACTATGCTAGTAAATCCA
AACTACAAGGCTATTCAACACCTGTCAAGAGCGTTCCCCTCGGTCACAGGGTTTATC
ACCCAAGTAACTTCGTCTTTTTCATCTGAGCTCTACCTCCGATTCTCCAAACGAGGGA
AGTTTTTCAGAGATGCTGAGTACTTGACCTCTTCCACCCTTCGAGAAATGAGCCTTGT
GTTATTCAATTGTAGCAGCCCCAAGAGTGAGATGCAGAGAGCTCGTTCCTTGAACTA
TCAGGATCTTGTGAGAGGATTTCCTGAAGAAATCATATCAAATCCTTACAATGAGAT
GATCATAACTCTGATTGACAGTGATGTAGAATCTTTTCTAGTCCACAAGATGGTTGA
TGATCTTGAGTTACAGAGGGGAACTCTGTCTAAAGTGGCTATCATTATAGCCATCAT
GATAGTTTTCTCCAACAGAGTCTTCAACGTTTCCAAACCCCTAACTGACCCCTCGTTC
TATCCACCGTCTGATCCCAAAATCCTGAGGCACTTCAACATATGTTGCAGTACTATG
ATGTATCTATCTACTGCTTTAGGTGACGTCCCTAGCTTCGCAAGACTTCACGACCTGT
ATAACAGACCTATAACTTATTACTTCAGAAAGCAAGTCATTCGAGGGAACGTTTATC
TATCTTGGAGTTGGTCCAACGACACCTCAGTGTTCAAAAGGGTAGCCTGTAATTCTA
GCCTGAGTCTGTCATCTCACTGGATCAGGTTGATTTACAAGATAGTGAAGACTACCA
GACTCGTTGGCAGCATCAAGGATCTATCCAGAGAAGTGGAAAGACACCTTCATAGG
TACAACAGGTGGATCACCCTAGAGGATATCAGATCTAGATCATCCCTACTAGACTAC
AGTTGCCTGTGAACCGGATACTCCTGGAAGCCTGCCCATGCTAAGACTCTTGTGTGA
TGTATCTTGAAAAAAACAAGATCCTAAATCTGAACCTTTGGTTGTTTGATTGTTTTTCTCAt
ttttgttgtttatttgttaagcgtGGGTCGGCATGGCATCTCCACCTCCTCGCGGTCCGACC
TGGGCATCCGAAGGAGGACGCACGTCCACTCGGATGGCTAAGGGAGagccagaaGGATCCG
GCTGCTAACAAAGCCCGAAAGGAAGCTGAGTTGGCTGCTGCCACCGCTGAGCAATA
ACTAGCATAACCCCTTGGGGCCTCTAAACGGGTCTTGAGGGGTTTTTTGCTGAAAGtC
GCGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAA
CCTGTCGTGCCAGCTGCATTAATGAATCGGCCAACGCGCGGGGAGAGGCGGTTTGC
GTATTGGGCGCTCTTaCGCTTCCTCGCTCACTGACTCGCTGCGCTCGGTCGTTCGGCT
GCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCACGC
TGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAAC
CCGGTAAGACACGACTTATCGCCACTGGCAGCAGCCACTGGTAACAGGATTAGCAG
AGCGAGGTATGTAGGCGGTGCTACAGAGTTCTTGAAGTGGTGGCCTAACTACGGCT
ACACTAGAAGGACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTTT
TTGTTTGCAAGCAGCAGATTACGCGCAGAAAAAAAGGATCTCAAGAAGATCCTTTG
ATCTTTTCTACGGGGTCTGACGCTCAGTGGAACGAAAACTCACGTTAAGGGATTTTG
GTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTA
ATCAGTGAGGCACCTATCTCAGCGATCTGTCTATTTCGTTCATCCATAGTTGCCTGAC
TCCCCGTCGTGTAGATAACTACGATACGGGAGGGCTTACCATCTGGCCCCAGTGCTG
CAATGATACCGCGAGACCCACGCTCACCGGCTCCAGATTTATCAGCAATAAACCAG
CCAGCCGGAAGGGCCGAGCGCAGAAGTGGTCCTGCAACTTTATCCGCCTCCATCCA
GTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCT
TCATTCAGCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTTGGCCGCA
GTGTTATCACTCATGGTTATGGCAGCACTGCATAATTCTCTTACTGTCATGCCATCCG
TAAGATGCTTTTCTGTGACTGGTGAGTACTCAACCAAGTCATTCTGAGAATAGTGTA
TGCGGCGACCGAGTTGCTCTTGCCCGGCGTCAATACGGGATAATACCGCGCCACATA
GCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGAT
CTTCAGCATCTTTTACTTTCACCAGCGTTTCTGGGTGAGCAAAAACAGGAAGGCAAA
ATGCCGCAAAAAAGGGAATAAGGGCGACACGGAAATGTTGAATACTCATACTCTTC
CTTTTTCAATATTATTGAAGCATTTATCAGGGTTATTGTCTCATGAGCGGATACATAT
TTGAATGTATTTAGAAAAATAAACAAATAGGGGTTCCGCGCACATTTCCCCGAAAA
GTGCCAC.

In some embodiments, the nucleotide sequence encoding GP38 has at least 80% sequence identity with SEQ ID NO: 2. In some embodiments, the nucleotide sequence encoding GP38 has at least 85%, at least 90%, at least 95%, at least 98%, at least 99% or 100% sequence identity with SEQ ID NO: 2. In some embodiments, the protein sequence of GP38 has a least 80% sequence identity with SEQ ID NO: 3. In some embodiments, the protein sequence of GP38 has at least 85%, at least 90%, at least 95%, at least 98%, at least 99% or 100% sequence identity with SEQ ID NO: 3.

In some embodiments, the nucleotide sequence encoding the mucin-like domain has at least 80% sequence identity with SEQ ID NO: 4. In some embodiments, the nucleotide sequence encoding the mucin-like domain has at least 85%, at least 90%, at least 95%, at least 98%, at least 99% or 100% sequence identity with SEQ ID NO: 4. In some embodiments, the protein sequence of the mucin-like domain has at least 80% sequence identitiy with SEQ ID NO: 5. In some embodiments, the protein sequence of the mucin-like domain has at least 85%, at least 90%, at least 95%, at least 98%, at least 99% or 100% sequence identity with SEQ ID NO: 5.

In some embodiments, the composition is therapeutically effective.

In some embodiments, the composition is immunogenic.

In some embodiments, the invention provides an isolated virion prepared from a host cell infected with the recombinant vector comprising a nucleotide sequence encoding at least one CCHFV glycoprotein and a mucin-like domain, incorporated into a rabies virus virion.

In some embodiments, the composition comprising a nucleotide sequence encoding at least one CCHFV glycoprotein and a mucin-like domain, incorporated into the rabies virus virion and further comprises a pharmaceutically acceptable excipient. In some embodiments, the pharmaceutically acceptable excipient is an adjuvant.

In some embodiments, a rabies-based vaccine, as disclosed in the instant invention, has several advantages in the competitive vaccine field because they provide many of the features required for a new vaccine. In some embodiments, as a killed vaccine, the vaccine is safe for all population groups. In other embodiments, long-term protection is expected since the rabies virus, (RABV) vaccine often provides life-long protection. For example, long term immunity has been shown in rodent models and in non-human primates. In other embodiments, the RABV vector can be stabilized at room temperature. In other embodiments, dual protection can be expected from a Rhabdoviral-based CCHFV vaccine construct, eliciting protection against Rhabovirus as well as CCHFV.

In some embodiments, GP38 is the main CCHFV glycoprotein antigen. In some embodiments, the GP38 protein offers protection in a viral-vectored vaccine. In some embodiments, the RABV virions are inactivated (killed virions). In some embodiments, the inactivated RABV virions are safe when administered to mammals. In some embodiments the inactivated RABV virions comprise a CCHFV-derived domain. In some embodiments, the inactivated RABV virion comprising the CCHFV-derived domain is safe when administered to mammals. In some embodiments, the mammal is a human.

In some embodiments, the RABV-based CCHFV vaccine elicits a humoral immune response. In some embodiments, the RABV-based CCHFV vaccine elicits a cellular immune response. In some embodiments, the RABV-based CCHFV vaccine elicits both a humoral and a cellular immune response.

In some embodiments, the immune response elicited by RABV-based CCHFV vaccine is primarily directed at GP38. In some embodiments, the immune response elicited by RABV-based CCHFV vaccine is directed only at GP38.

In one aspect, the disclosure provides a therapeutically effective amount of a composition comprising a recombinant vector from an attenuated rabies virus comprising a nucleotide sequence encoding at least one CCHFV glycoprotein and at least one chaperone protein which are incorporated into the rabies virus virion, wherein the rabies virus genome is attenuated and, wherein the therapeutically effective amount of said recombinant rabies virus vector is an amount sufficient to induce an immune response. Without meaning to be limited by theory, the chaperone protein promotes the native folding of the CCHFV glycoprotein and thereby enhances the utility of the composition when used to condition an immune response in a subject.

In some embodiments, the invention provides a composition comprising a therapeutically effective amount of a recombinant rabies virus vector comprising a nucleotide sequence encoding at least one CCHFV glycoprotein and at least one chaperone protein affecting cell surface expression of the CCHFV glycoprotein, wherein the virus vector is incorporated into the rabies virus virion.

As used herein, a “chaperone protein” is a protein which assists in protein folding and processing (i.e. glycosylation) in the cell. Chaperones share the ability to recognize and bind nonnative proteins, thereby preventing non-specific aggregation. Chaperones may also assist in the conformational folding or unfolding and the assembly or disassembly of other macromolecules. Chaperones may belong to the family of heat shock proteins, since the tendency to aggregate increases under conditions of stress. Exemplary heat shock proteins are HSP47, HSP60, HSP70, HSP90 and HSP100. Other proteins with chaperone like functions GRP78/BiP, GRP94, GRP170, alnexin, calreticulin, ERp29. In some embodiments, a protein with chaperone-like properties is MLD.

In some embodiments, the rabies virus vector is derived from a live attenuated SAD B19 RABV vaccine.

In some embodiments, the immune response is primarily mediated through non-neutralizing antibodies. In some embodiments, the immune response is directed against at least one CCHFV virus protein. In some embodiments, the immune response is protective against at least one CCHFV virus protein. In some embodiments, the immune response is directed against at least one rabies virus protein and at least one CCHFV virus protein.

In some embodiments, the polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

In some embodiments, a RNA polynucleotide of an RNA (e.g., mRNA) vaccine encodes one or more antigenic polypeptides. In some embodiments, the one or more of the antigenic polypeptides is glycosylated. In some embodiments, the antigenic polypeptide is GP38, or a fragment thereof. In some embodiments, a RNA (e.g., mRNA) encodes one or more antigenic polypeptides of a CCHFV virus. In some embodiments, the one or more polypeptides are GP38 and a mucin-like domain (MLD).

In one aspect the disclosure provides a CCHFV ribonucleic acid (RNA) polynucleotide vaccine, comprising at least one RNA having an open reading frame encoding at least one CCHFV glycoprotein or an immunogenic fragment thereof and a mucin-like domain, and a pharmaceutically acceptable excipient.

### Methods

In one aspect, the invention provides a method of conditioning an immune response protective against a CCHFV virus in a subject, the method comprising administering to the subject a recombinant vector from an attenuated rabies virus comprising a nucleotide sequence encoding at least one CCHFV glycoprotein and a mucin-like domain.

In some embodiments, the-immune response is directed against a CCHFV glycoprotein.

In some embodiments, the immune response is directed against a CCHFV glycoprotein and a rabies protein. In some embodiments, the immune response directed against the CCHFV glycoprotein primarily comprises non-neutralizing antibodies.

In one aspect, the invention provides a method of treating a subject infected with a CCHFV virus, comprising administering to the subject a composition comprising a recombinant vector from an attenuated rabies virus that expresses at least one CCHFV immunogenic protein and a mucin-like domain, wherein said composition induces an effective immune response against one or both of said viruses wherein the at least one immunogenic CCHFV protein comprises the amino acid sequence of SEQ ID NO: 3.

In some embodiments, the viral glycoproteins interact with host cells to mediate viral entry, although the exact mechanisms of viral entry is unclear. CCHFV glycoproteins induce production of neutralizing and non-neutralizing antibodies in vivo. It has been shown that GP38 is required for eliciting protection in mice immunized with DNA vaccines containing the CCHFV glycoproteins.

In some embodiments the RABV-based CCHFV vaccine when administered to a subject induces the production of neutralizing antibodies. In other embodiments, the RABV-based CCHFV vaccine when administered to a subject induces the production of non-neutralizing antibodies. In some embodiments, the RABV-based CCHFV vaccine induces the production of both neutralizing and non-neutralizing antibodies. In some embodiments, the non-neutralizing antibodies are more effective than virion-neutralizing antibodies in the protection against CCHFV.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook.

### EXAMPLES

The invention is now described with reference to the following Examples. These Examples are provided for the purpose of illustration only, and the invention is not limited to these Examples, but rather encompasses all variations that are evident as a result of the teachings provided herein.

Specifically, the Examples section describes two related studies: a first study described in Example 1, and a more comprehensive second study described in Example 2.

### Example 1-1: Materials and Methods

### Virion Propagation and Validation

All viruses containing CCHFV-coM (human condon optimized CCHFV M gene), GP38 or GP85 were recovered on 293T cells using a well-established plasmid-based reverse genetics approach. These viruses were then propagated on Vero E6 cells and surface expression of both RABV and CCHFV glycoproteins was verified through immunofluorescence of cells infected at a low multiplicity of infection (MOI), 0.01 and fixed in paraformaldehyde (pfa) 72 hrs after infection for RABV-based viruses and 24 hrs after infection for VSV-based viruses. Glycoprotein incorporation into virions was confirmed through western blot and SDS PAGE protein gel of purified virions.

### In Vitro Characterization of Viruses

Viruses containing CCHFV-coM were recovered using a well-established plasmid-based reverse genetic approach. Infectious viral titers of RABV-based vectors are determined using a focus-forming assay adapted from established methods and titers of VSV-based vectors by a well-established plaque assay. Upon recovery, viruses were characterized through immunofluorescence of infected cells, Western blot and SYPRO® Ruby protein gel stain of purified viral particles to confirm cell surface expression and virion incorporation of RABV and CCHFV glycoproteins. To further confirm the presence of these glycoproteins on the virion, transmission electron micrographs will be performed as previously described. To quantify glycoprotein incorporation, surface expression of CCHFV and RABV glycoproteins on infected cells were analyzed through flow cytometry, as previously described. Biotinylation assays will also be used to further quantify the amount of glycoprotein incorporated into the plasma membrane, as previously described. The effects of CCHFV glycoproteins on virus production was assessed through a one-step growth curve with infection at a high multiplicity of infection (MOI) of 10 and assess the effects on the speed of viral replication as well as viral spread through a multi-step growth curve with infection at a low MOI of 0.01. The parental vectors, BNSP333 and/or VSV-GFP are used as controls for these studies.

### Immunogenicity Studies:

Viruses will be concentrated and purified by sucrose cushion and virus particles will be inactivated with β-propriolactone (BPL). Inactivation will be confirmed as previously described. To assess vaccine immunogenicity, groups of C57BL/6 or interferon-α/β receptor knockout (IFNAR−/−) mice were immunized intramuscularly in the hind limb with 10 μg of BPL inactivated recombinant viruses with or without 5 μg of toll-like receptor 4 agonist, Synthetic Monophosphoryl Lipid A in SE, a squalene-based oil-in-water emulsion, on days 0 and 28. For comparison to previously tested vaccines, a control group will be immunized with the Bulgaria vaccine, an inactivated CCHFV particle grown on mouse brains shown to be protective in mice. However, it should be noted that it is currently unknown how this vaccine protects. The Bulgaria vaccine will be administered intramuscularly as a 20 μg dose adjuvanted with alum as previous described and the mRNA M segment vaccine will be administered as a 10 μg dose. Sera was collected on days 0, 7, 14, 28, 35, and 56 post-immunization and assayed by indirect ELISA for presence of total immunoglobulin G (IgG) antibodies against both RABV and CCHFV glycoproteins. Recombinant RABV glycoproteins was purified from the heterologous vector (i.e. for ELISAs on sera from mice immunized with a recombinant RABV, antigen purified from VSV will be used and visa-versa) as previously described, to avoid cross-reactivity against viral vector proteins. C-terminal HA-tagged CCHFV-Gc antigen was prepared as previously described. C-terminal strep-tagged CCHFV-GP38 antigen was prepared as previously described. At day 56 post-immunization mice underwent cardiac puncture for a final blood draw and were euthanized humanely. A long-term immunogenicity study will also be performed following the same schedule described above but collecting sera and sacrificing mice one year after the first immunization. The same ELISA assays will be carried out as described above.

### Example 1-2: Design of a Recombinant Rabies Virus (RABV) Expressing Chimeric CCHFV

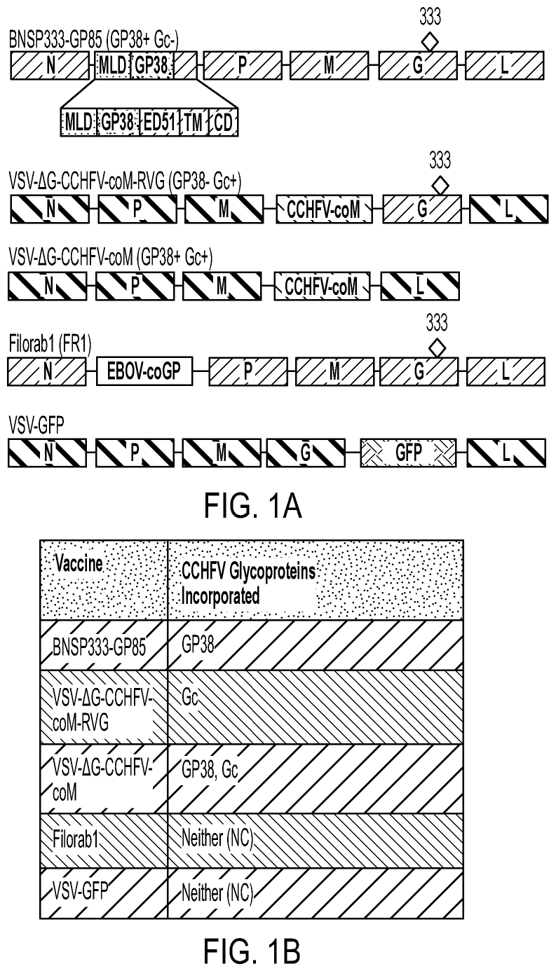

A recombinant Rabies Virus (RABV) expressing a chimeric CCHFV GP38 (BNSP333-GP85, also sometimes referred to as BNSP333-MLD-GP38-RVG, BNSP333-k-MLD-GP38-RVG, or BNSP333-GP85-ED51), was designed which has the CCHFV signal sequence, the mucin-like domain (MLD) of CCHFV, GP38 and 51 amino acids of the ectodomain, transmembrane domain and cytoplasmic domain of RABV glycoprotein (G). Inclusion of the MLD ensures proper folding and processing of GP38. This chimeric GP38 gene has been placed between the nucleoprotein (N) and phosphoprotein (P) genes in the rabies virus genome for optimal incorporation into the virion (FIG. 1A-1B). When the virus is recovered, GP38 is incorporated into the RABV virion and expressed on the surface. The virus is inactivated for use as a killed vaccine, where it elicits antibodies directed towards CCHFV GP38.

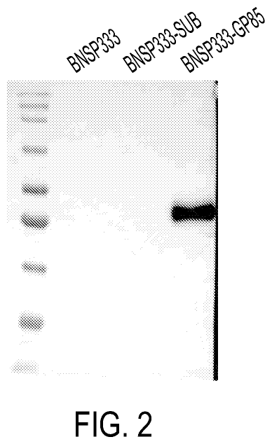

FIG. 2 depicts a Western blot of the characterization of the BNSP333-GP85 Virus construct. Sucrose-purified virions were run on an SDS PAGE protein gel. Viruses analyzed are the parental BNSP333 vector, a recombinant RABV with an irrelevant protein subolesin (SUB) or BNSP333-GP85. The blot was probed with monoclonal antibody 13G8, which is specific for CCHFV GP38.

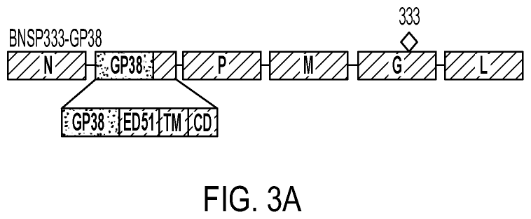

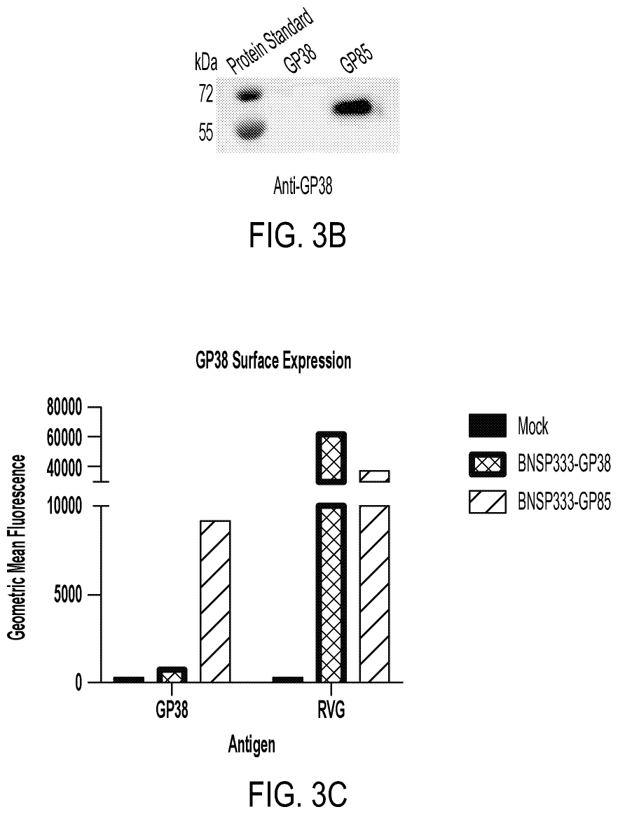

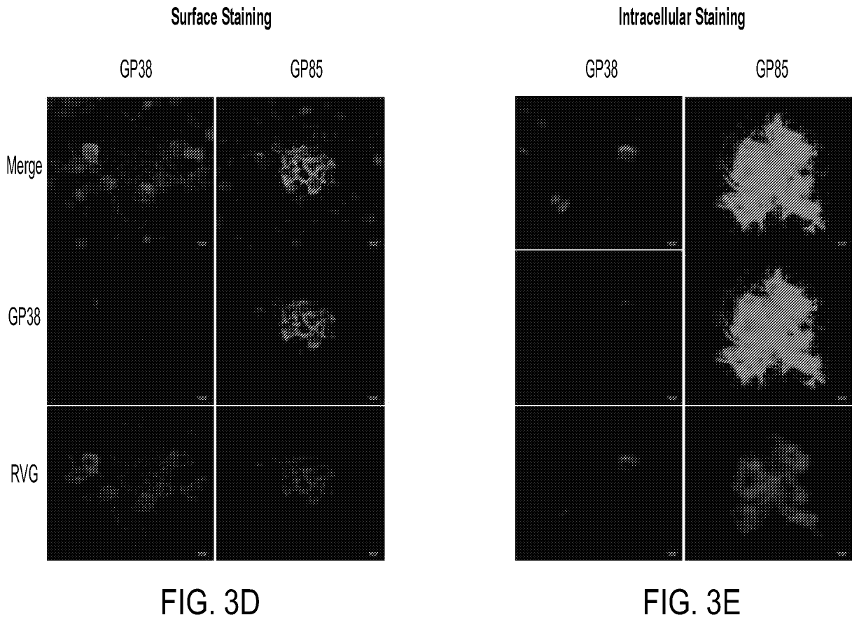

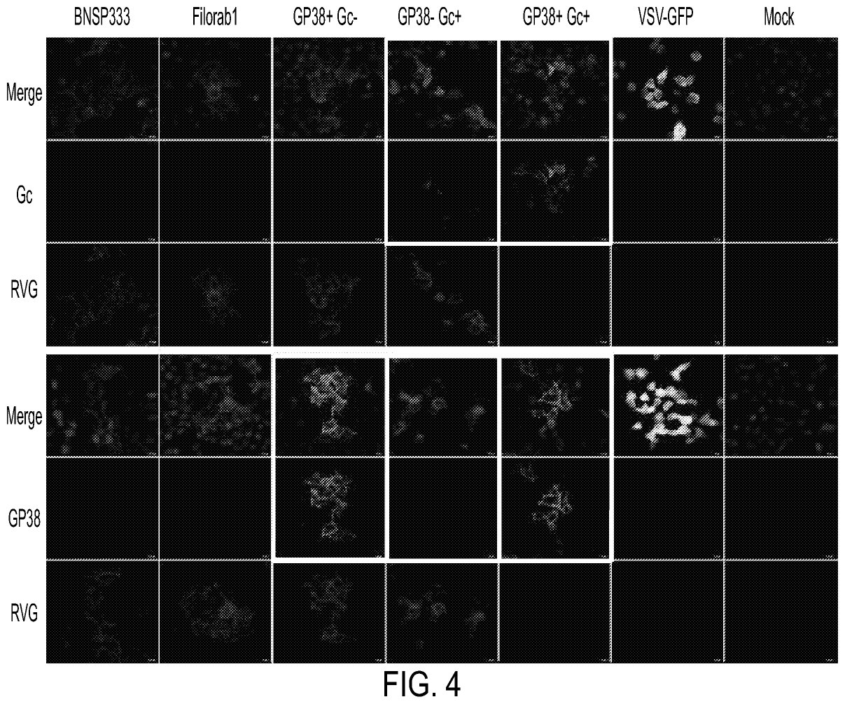

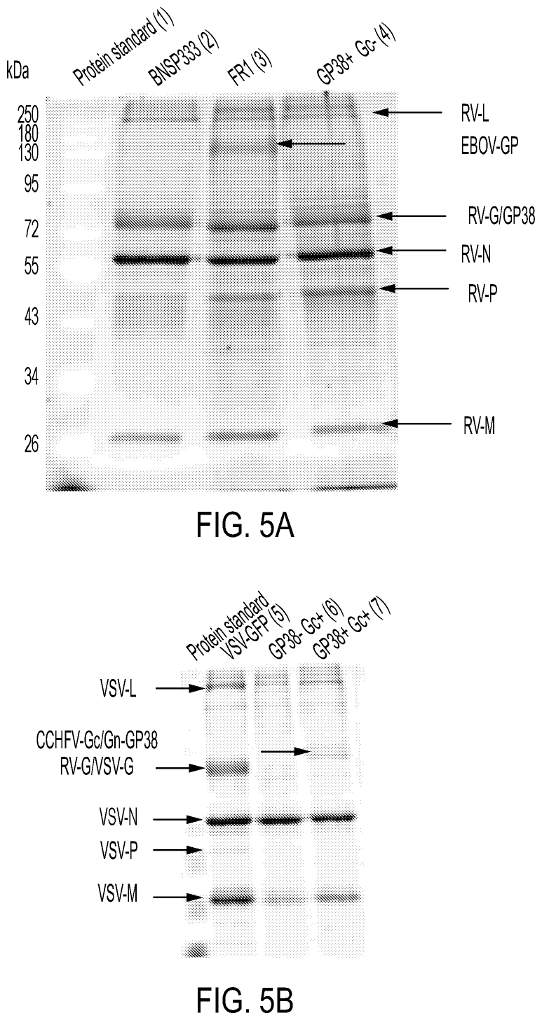

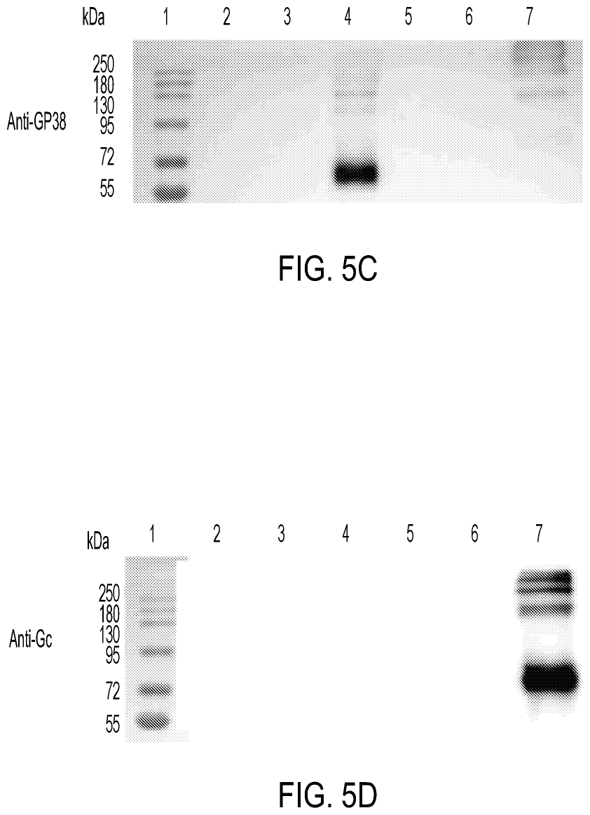

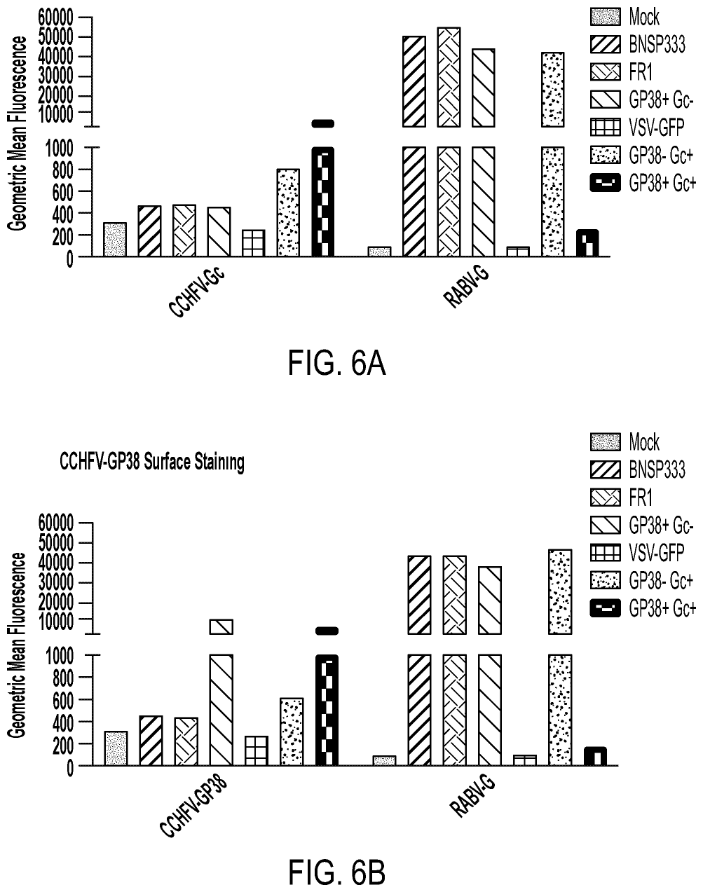

Another construct, BNSP333-GP38, was designed lacking the MLD domain (FIG. 3A). FIG. 3B shows the immunoblotting results with an anti-GP38 antibody, and FIG. 3C compares the GP38 surface expression in the GP38 and GP85 constructs. In FIGS. 3D and 3E, surface staining and intracellular staining, respectively, are compared in cells infected with the GP38 and GP85 constructs. FIG. 4 depicts fluorescent immunohistochemistry detection of surface antigens of infected cells showing that they express the expected glycoproteins. FIG. 5A-5D depict SDS-PAGE and immunoblots showing that the vaccine viruses express the expected glycoproteins. FIG. 6A-6B depict CCHFV-Gc surface staining (FIG. 6A) and CCHFV-GP38 surface staining (FIG. 6B) in cells mock transformed or infected with the six constructs (BNSP333, FR1, GP38+ Gc−, VSV-GFP, GP38− Gc+, and GP38+ Gc+).

### Example 1-3: Use of the Live Vaccine in an Immunogenicity Experiment

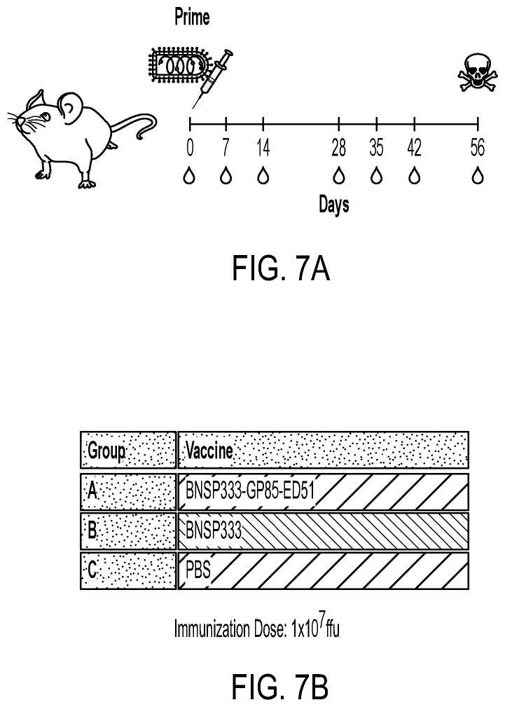

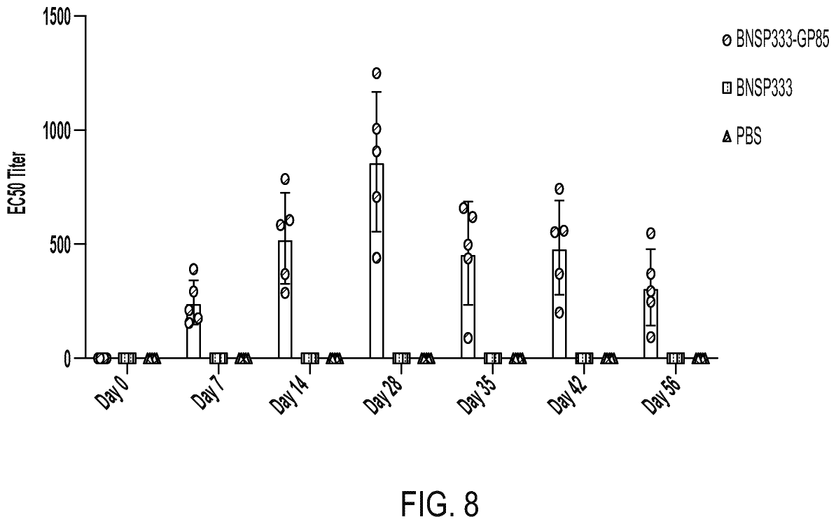

Mice were immunized with 1×10⁷pfu of GP38+ Gc−, GP38+ Gc+, GP38− Gc+ or FR1 (FIG. 7A-7B). All mice showed a robust antibody response against GP38 as shown through anti-GP38 ELISA for total IgG (FIG. 8).

### Example 1-4: Use of the Dead Vaccine in an Immunogenicity Experiment

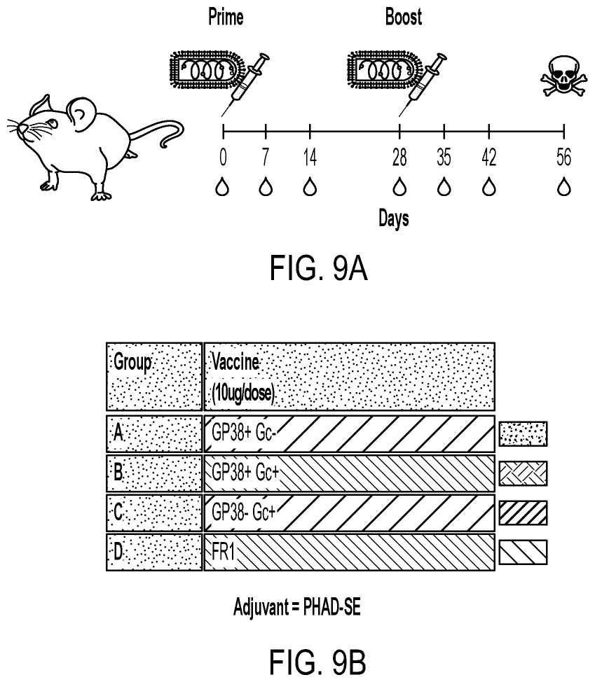

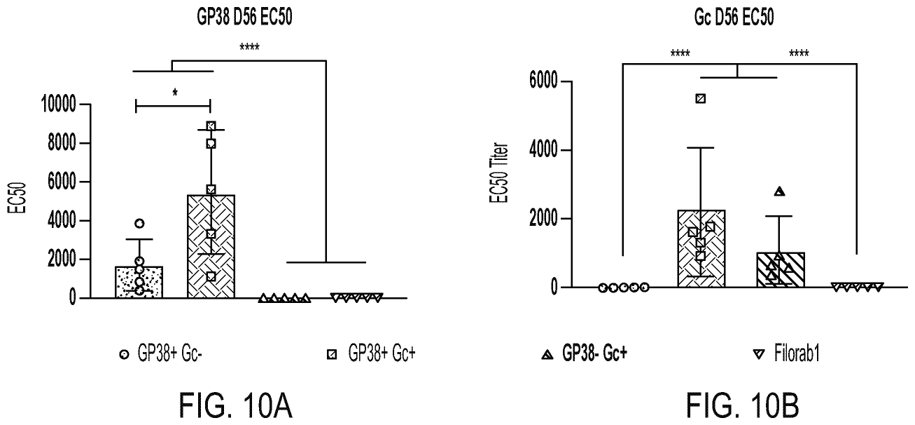

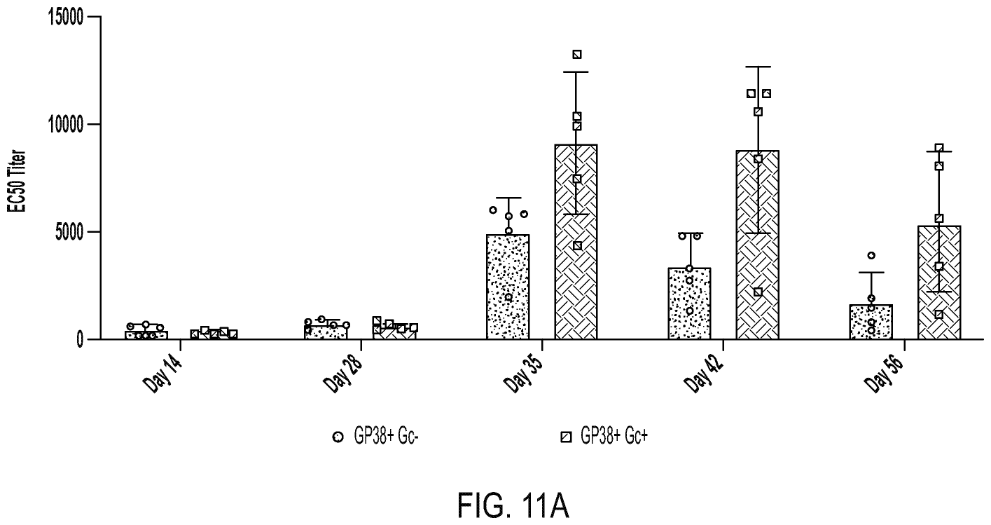

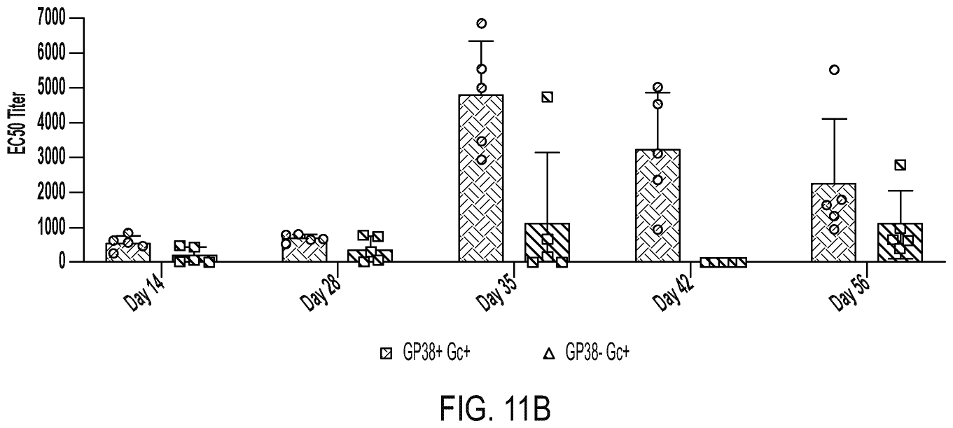

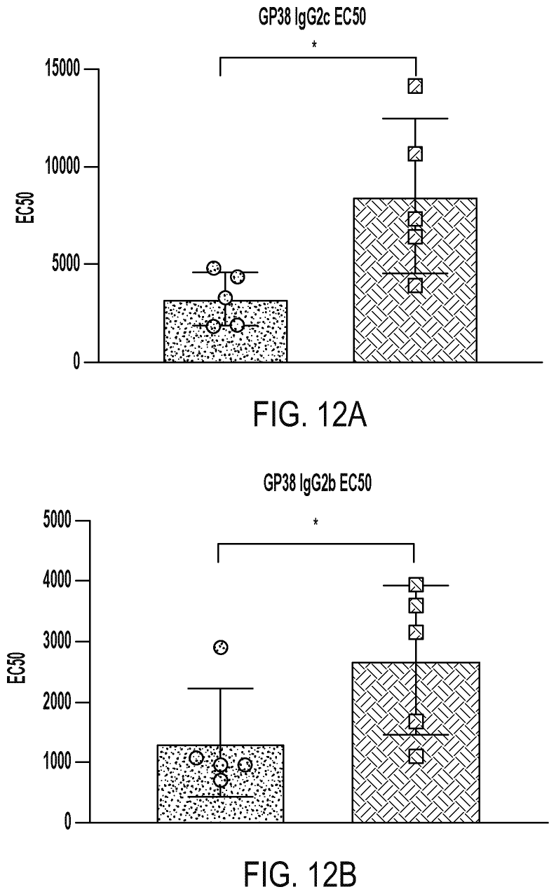

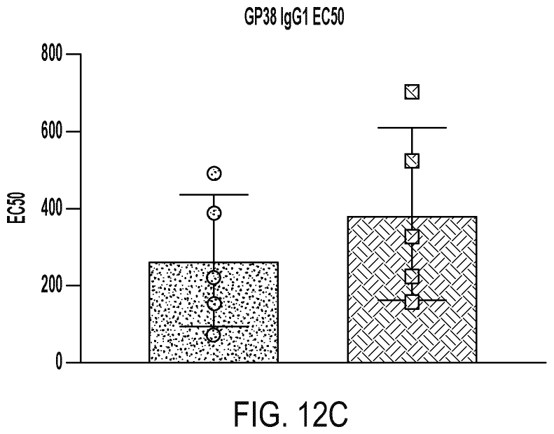

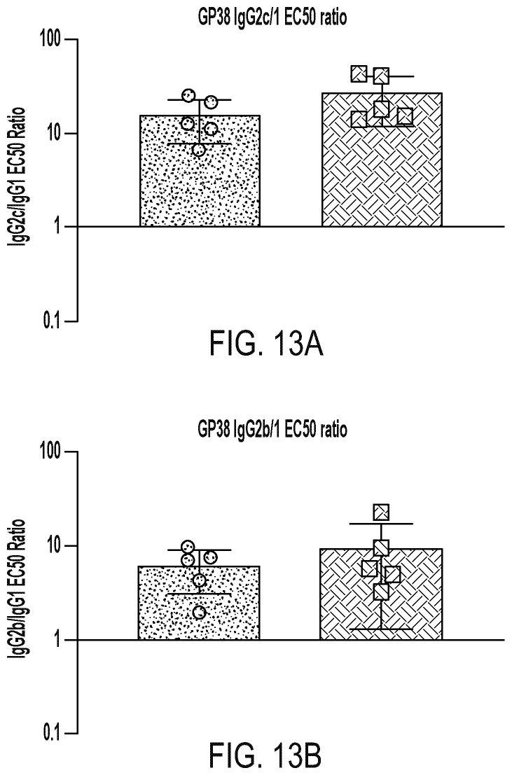

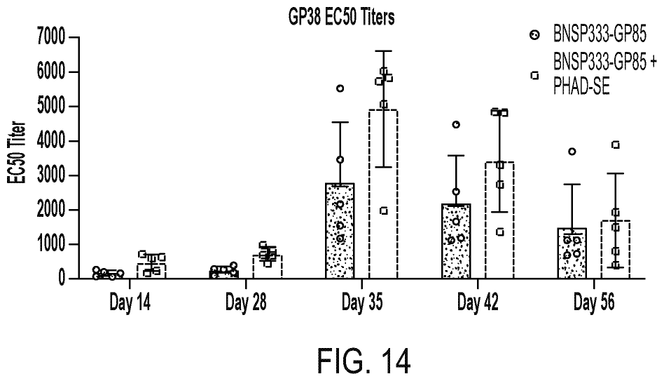

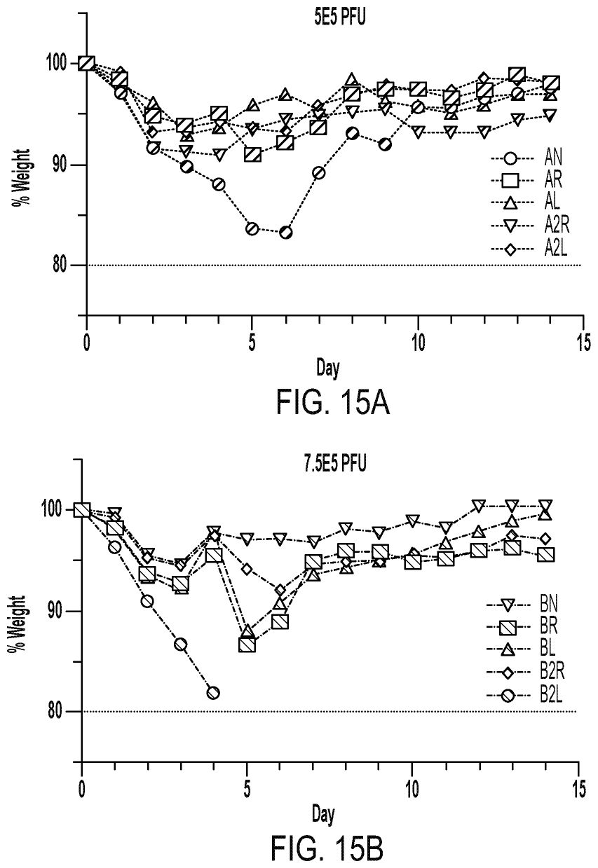

The virus was then inactivated and used in as a killed vaccine in an immunogenicity experiment. Mice were immunized on days 0 and 28 with 10 μg of vaccine with or without adjuvant (PHAD-SE) (FIG. 9A-9B). FIG. 10A-10B depict the EC₅₀ values of the anti-GP38 antibody titers (FIG. 10A) and the anti-Gc antibody titers (FIG. 10B), and the EC₅₀ values of the respective antibodies over time (FIGS. 11A and 11B, respectively) of the adjuvanted groups. FIG. 12 depict the EC₅₀ values of the various isotype antibodies elicited upon immunization with GP38+ Gc− and GP38+ Gc+, and FIG. 13 depict the isotype ratios of algG2c/1 and IgG2b1 elicited upon immunization with GP38+ Gc− and GP38+ Gc+. All mice receiving the BNSP333-GP85 vaccine, regardless of whether or not the immunization included adjuvant, showed a robust antibody response against GP38, which a boosted response after the second immunization, as shown through anti-GP38 ELISA for total IgG (FIG. 14).

### Example 1-5: Alternative challenge models for CCHFV

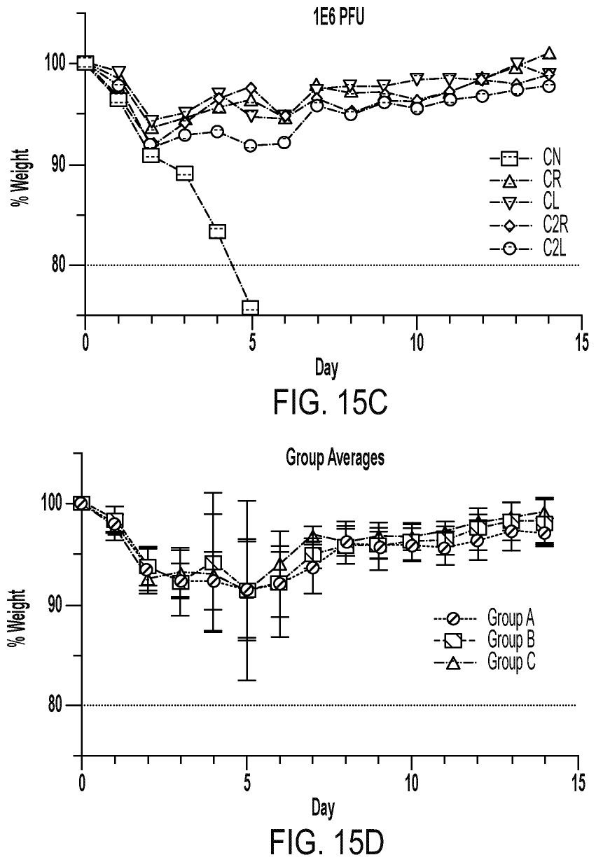

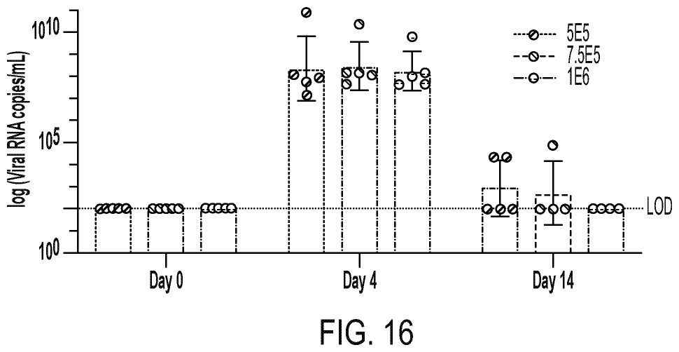

Mice were challenged with 5×10⁵ pfu, 7.5×10⁵ pfu, or 1×10⁶ pfu a surrogate challenge virus for CCHFV (a vesicular stomatitis virus with its glycoproteins replaced with the CCHFV glycoproteins, VΔGcoM). The weight curves of these challenge experiments show that virus VΔGcoM is pathogenic in IFNAR−/−mice; FIG. 15A, mice challenged with 5×10⁵ pfu, FIG. 15B, mice challenged with 7.5×10⁵ pfu; FIG. 15C, mice challenged with 1×10⁶ pfu. FIG. 15D depicts group averages. The log viral RNA copy numbers in these groups on day 0, 4, and 14 are shown in FIG. 16, showing that surrogate challenge virus causes high levels of viremia.

### Example 1-6: Evaluation of Vaccine Efficacy

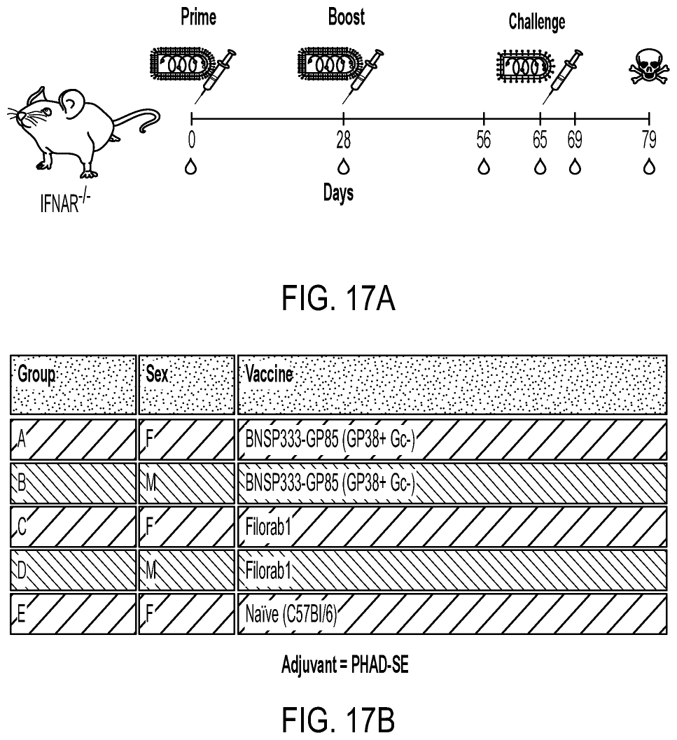

Finally, this vaccine efficacy was tested in a challenge experiment. Interferon α/β receptor 1 knockout mice were immunized on days 0 and 28 with 10 μg of vaccine with adjuvant (PHAD-SE). Mice were then challenged on day 65 with 5×10⁵ pfu of VΔGcoM (FIG. 17). Weight was measured over the course of 2 weeks and viremia measured at days 0, 4 and 14 post infection.

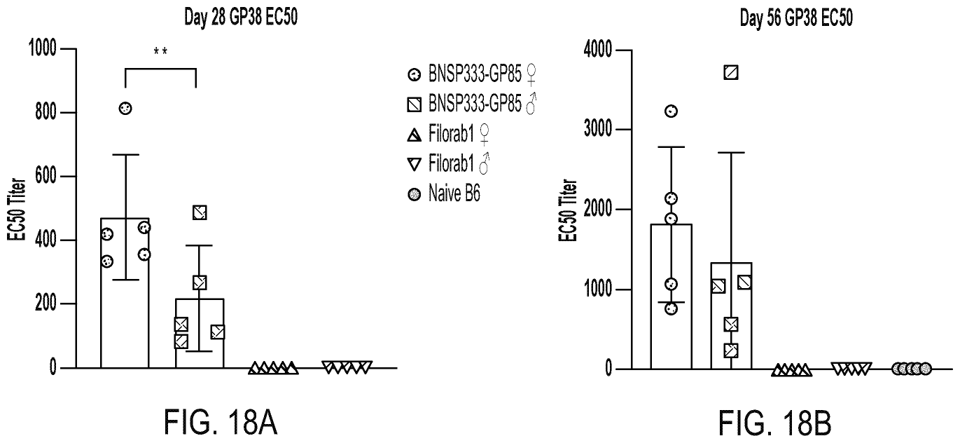

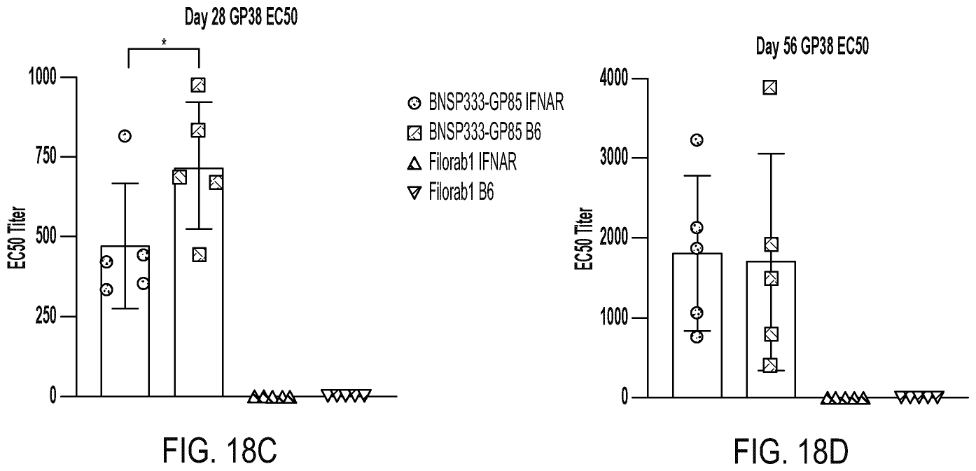

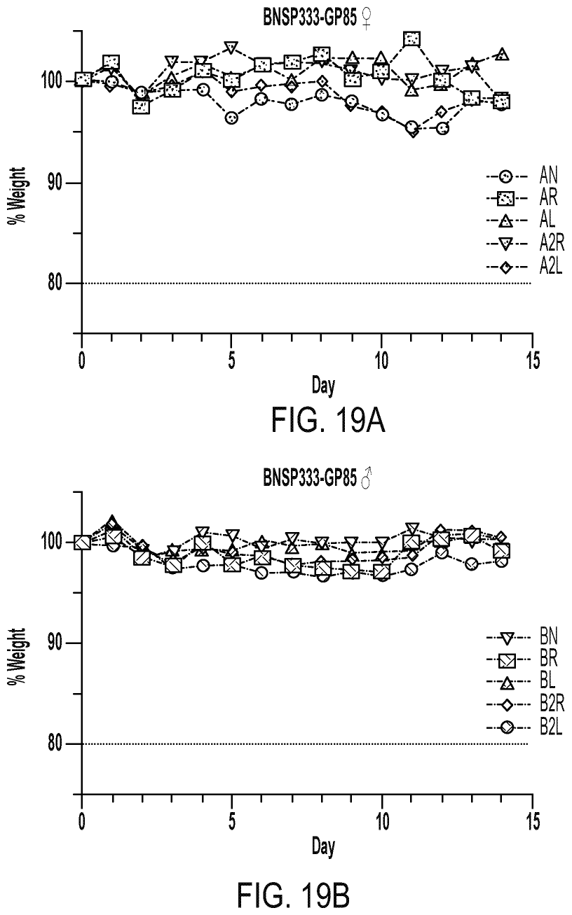

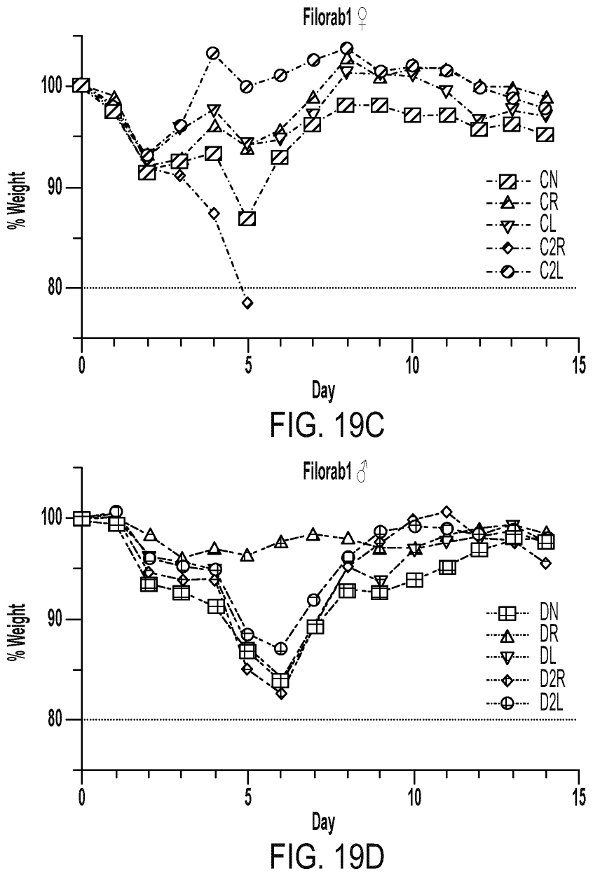

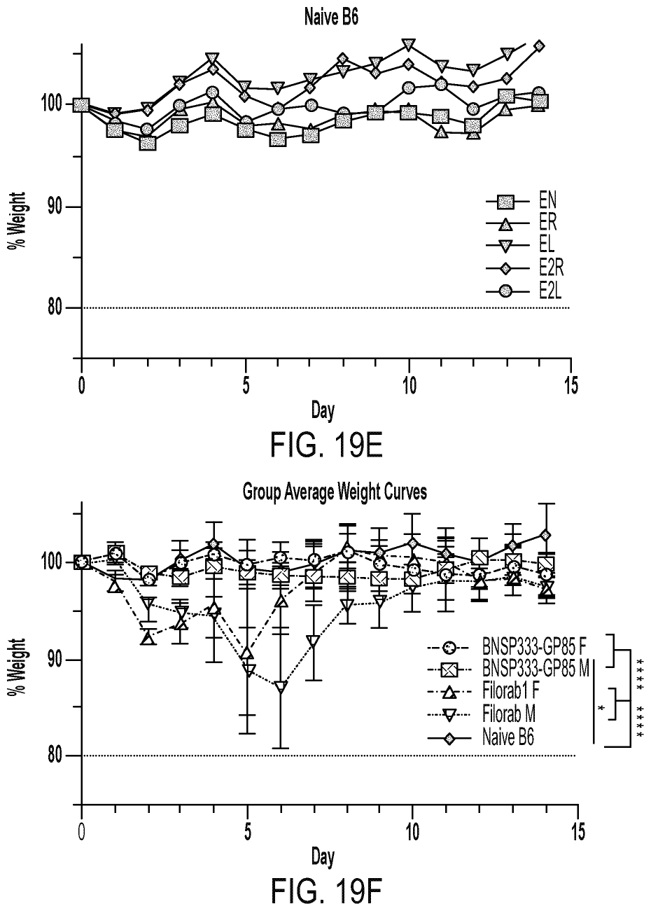

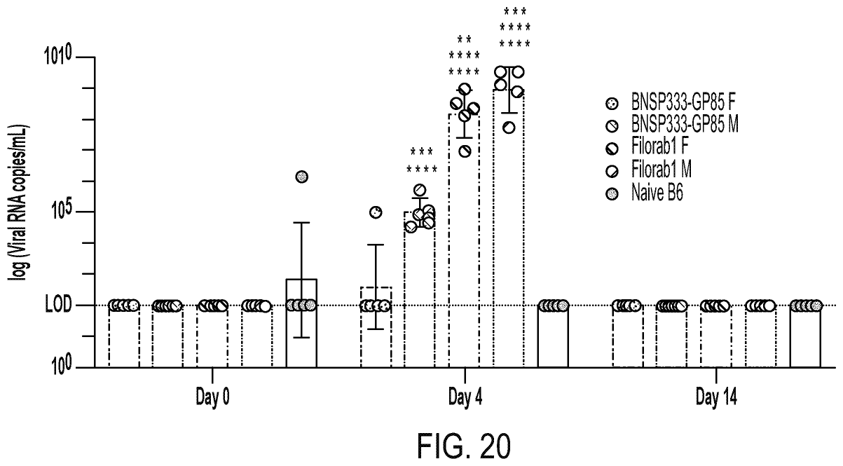

FIG. 18A-18D depict the EC₅₀ values of the anti-GP38 antibody titers after immunization with constructs as shown in (FIG. 18A-18B), and in (FIG. 18C-18D). FIG. 18C-18D compares anti-GP38 antibody titers in IFNAR−/− and wildtype mice. FIG. 19A-19F depict the weight curves of mice challenged; FIG. 19A, mice immunized with BNSP33-GP85 (females), FIG. 19B, mice immunized with BNSP33-GP85 (males); FIG. 19C, mice immunized with Filorab1 (females); FIG. 19D, mice immunized with Filorab1 (males); FIG. 19E, unimmunized naïve B6 mice (wildtype); FIG. 19F, group average weight curves. FIG. 20 depicts the log viral RNA copy number over time in female mice immunized with BNSP33-GP85; male mice immunized with BNSP33-GP85, female mice immunized with Filorab1; male mice immunized with Filorab1, and in unimmunized naïve B6 mice (wildtype). Results show that mice immunized with BNSP333-GP85 showed no significant weight loss and a significant reduction in viremia compared with controls immunized with a vaccine against an irrelevant glycoprotein (filorab1).

### Example 2: GP38 as a Vaccine Target for Crimean-Congo Hemorrhagic Fever Virus

Crimean-Congo Hemorrhagic Fever Virus (CCHFV) is a tick-borne virus that causes severe hemorrhagic disease in humans. There is a great need for effective vaccines and therapeutics against CCHFV for humans, as none are currently internationally approved. Recently, a monoclonal antibody against the GP38 glycoprotein protected mice against lethal CCHFV challenge. To show that GP38 is required and sufficient for protection against CCHFV, the present study used three inactivated rhabdoviral-based CCHFV-M vaccines, with or without GP38 in the presence or absence of the other CCHFV glycoproteins. All three vaccines elicited strong antibody responses against the respective CCHFV glycoproteins. However, only vaccines containing GP38 showed protection against CCHFV challenge in mice; vaccines without GP38 were not protective. The results of this study establish the need for GP38 in vaccines targeting CCHFV-M and demonstrate the efficacy of a CCHFV vaccine candidate based on an established vector platform.

Crimean-Congo Hemorrhagic Fever Virus (CCHFV) is an emerging infectious disease with an extensive global distribution spanning across areas of Africa, Asia, the Middle East, and Europe. The wide range of endemic areas is due to the natural habitat of CCHFV's tick vector, ticks of the Hyalomma genus. Areas where this tick can survive are increasing due to anthropogenic factors such as habitat modification, thus increasing the areas where CCHFV can circulate. CCHFV infects a wide range of mammalian hosts, yet it does not cause visible disease in these animals. However, CCHFV can cause Crimean-Congo hemorrhagic fever (CCHF) in humans, which first presents with flu-like symptoms and progresses to bleeding, petechiae, and, in more severe cases, organ failure and death. The case-fatality rate for CCHF is up to 40%, and there are no licensed CCHFV-specific vaccines or treatments available for humans. Therefore, CCHFV is designated as a biosafety level 4 (BSL-4) pathogen, further highlighting the need for effective vaccines and therapeutics. Accordingly, CCHFV is classified as an NIH/NIAID Category A and World Health Organization (WHO) high-priority pathogen.

There have been a variety of vaccine strategies against CCHFV tested in animal models with varying success. The only vaccine ever tested in humans was a whole inactivated virus vaccine propagated in mouse brains that reduced cases in Bulgaria, but requires BSL-4 laboratories for production and is administered as a four-dose regimen. While many other strategies have proven to be protective in animal models, there are concerns regarding the clinical application of each candidate. A cell culture produced whole inactivated virus vaccine showed 80% protection in mice; however, it requires a BSL-4 facility for production, which is dangerous and expensive. DNA vaccines using both the nucleoprotein(S) and glycoprotein (M) genes, individual glycoproteins (GN and Gc) or a combination of these antigens have demonstrated 100% protection in mice or Cynomolgus macaques, but DNA vaccines have not been effective in humans. A nucleoside-modified mRNA vaccine using CCHFV nucleoprotein and/or glycoproteins also showed 100% protection in mice. However, the study did not investigate the longevity of the immune responses elicited by the vaccine, which might be a problem based on the findings of waning humoral immune response to the Severe Acute Respiratory Syndrome Coronavirus 2 (SARS-COV-2) mRNA vaccine. Finally, both live Modified Vaccinia Ankara (MVA) and Vesicular Stomatitis virus (VSV) vaccines containing the CCHFV-M gene protected mice from CCHFV challenge, but supporting clinical studies are pending. While live vaccine strategies can be effective, there is always a concern about the virulence (whether inherent or mutation acquired) of these vectors, especially when used in immunocompromised people, pregnant women, and children. Thus, there is still a great need for an effective and safe CCHFV vaccine strategy.

Rhabdoviruses, specifically rabies virus (RABV) and VSV, have been used as vaccine vectors for a variety of infectious diseases. These vectors have many advantages, including their small, easily manipulated genome that can stably express foreign glycoproteins and their well-established safety profiles. Both vectors can be used as inactivated vaccines that will elicit immune responses against both foreign glycoproteins and the native rhabdoviral glycoproteins; however, VSV has never been tested as a killed vector. The RABV vaccine has been shown to elicit long-lasting immunity in humans, which is important for a vaccine platform. Moreover, a rabies-based vaccine against SARS-COV-2 is currently being evaluated in humans. Finally, RABV and CCHFV share many endemic regions, and thus a bivalent vaccine against both viruses would have a significant impact in the affected areas.

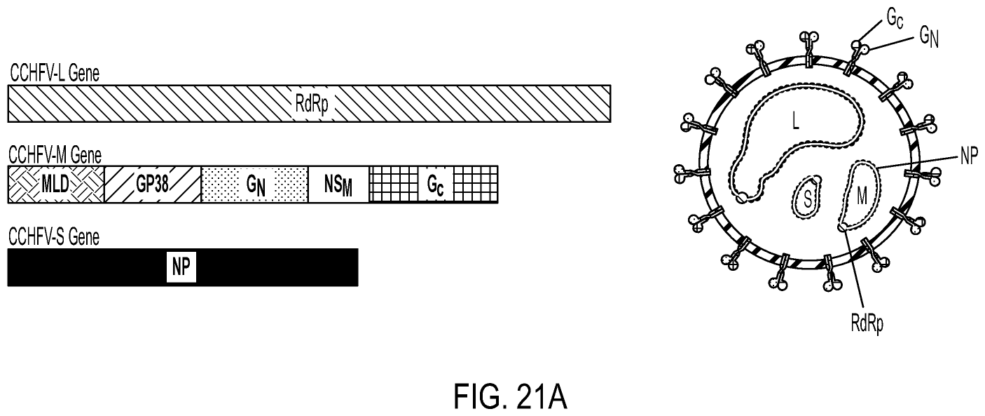

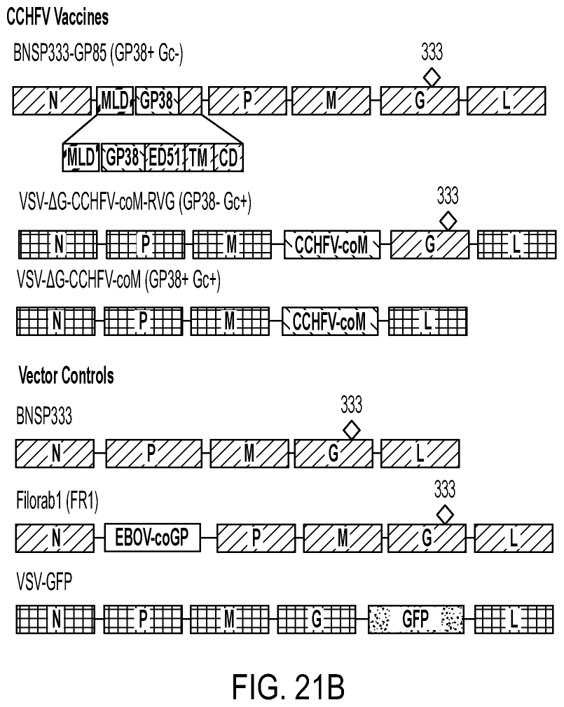

CCHFV is a member of the order Bunyavirales, family Nairoviridae, a group of single-stranded negative-sense RNA viruses with tri-segmented genomes. Vaccine strategies targeting the CCHFV M segment have shown protection in mouse challenge models. This gene encodes for the virus's glycoproteins, specifically structural proteins GN and Gc, secreted GP38, and non-structural proteins NSM and a mucin-like domain (MLD) (FIG. 21A). GN and Gc are embedded in the membrane that encompasses the virion and mediate cell attachment and entry (FIG. 21A), and GN is suspected of playing a role in virion assembly. GP38 and the MLD, referred to as GP85, play a role in the processing and trafficking of the structural glycoproteins and are indispensable for viral replication. NSM plays a role in Gc processing but is not required for viral replication.

Currently, there are no defined correlates of protection for CCHFV. Studies using either part of or the full-length CCHFV-M gene as a vaccine target have shown varying results regarding the vaccine's protective efficacy. Specifically, vaccines that induce immune responses against the full-length CCHFV-M are protective, while those that only target the structural proteins are not protective against CCHFV. The humoral immune response elicited against CCHFV-M during natural infection is specific for Gc and GP38. Interestingly, although the Gc antibodies are neutralizing, they are not always protective. GP38 antibodies are non-neutralizing, and one monoclonal antibody was shown to be protective in an adult mouse challenge model. Additionally, a CCHFV-M based DNA vaccine study showed that GP38 was required for protection. Thus, GP38 is a very attractive target antigen for a CCHFV vaccine that has not been extensively tested in the absence of other CCHFV glycoproteins.

Here, the present study presents a novel approach to an effective CCHFV vaccine based on RABV virions containing membrane-anchored GP38. To demonstrate the requirement of immune responses against GP38 for protection against CCHFV, the present study developed two VSV-based inactivated CCHFV vaccines containing the full M segment with or without GP38. Efficacy of the novel vaccine was shown in two animal models: a non-BSL-4 VSV-based surrogate challenge model for CCHFV in immunocompromised interferon α/β receptor 1 knockout (IFNAR^{-/-}) mice, and challenge with wildtype CCHFV in transiently immune suppressed C57BL/6 mice. The results indicate that immune responses against GP38 are required for protection against CCHFV and that the GP85 vaccine is an excellent candidate for a CCHFV vaccine.

### Example 2-1: Vaccine Design

To construct the rhabdoviral-based CCHFV vaccines, the present study used the rabies vector BNSP333 and VSV vector cVSV-XN. BNSP333 is a well-characterized vector derived from RABV vaccine strain SAD-B19. SAD-B19 has been further attenuated through an arginine to glutamic acid mutation at amino acid 333 of the glycoprotein (G) gene, which reduces the vector's neurotropism. cVSV-XN is based on the Indiana strain of VSV, which is attenuated by an unknown mechanism. A human-codon optimized CCHFV-M (coM) gene from strain IbAr10200 was used as the antigen for these vaccines. Three different CCHFV vaccines were constructed with an emphasis on GP38, which we hypothesize is required for a protective CCHFV vaccine (FIG. 1B). BNSP333-GP85 (GP38+ Gc−) contains a modified GP85, where CCHFV GP38 is anchored in the RABV virion by the addition of 51 amino acids of the RABV glycoprotein (G) ectodomain (ED), the transmembrane domain™ and cytoplasmic tail (CT), as used to incorporate other proteins into RABV virions. Since the CCHFV MLD is cleaved and secreted during glycoprotein maturation, the GP38 part is the only protein from CCHFV M present in this vaccine (FIGS. 2E-2F). The second construct, VSV-AG-CCHFV-coM-RVG (GP38− Gc+), is a VSV-vectored vaccine containing the full M gene with the terminal 50 amino acids in the Gc cytoplasmic tail truncated to allow the glycoproteins to traffic to the plasma membrane and RABV-G with the 333 attenuating mutation replacing VSV-G. CCHFV M gene expressed by this vector does not contain GP38 in its virion because GP38 is cleaved from GN and secreted from the cell; thus, this vaccine is a negative control for the role of GP38-mediated protection. Lastly, VSV-AG-CCHFV-coM (GP38+ Gc+) contains the same modified version of the M gene as GP38− Gc+ but lacks its own VSV glycoprotein and incorporates GP38 into the virion due to a mutation in the cleavage site between GP38 and GN. Therefore, the GP38+ Gc+ vaccine is a positive control for GP38-mediated protection.

All viruses were recovered, passaged twice and sequenced. The GP38+ Gc+ virus developed two mutations, L517R and L518S, in the cleavage motif between GP38 and GN, as mentioned above, and the GP38+ Gc− and GP38−Gc+ viruses did not acquire any mutations.

### Example 2-2: Incorporation of CCHFV Glycoproteins into Rhabdoviral Vectors

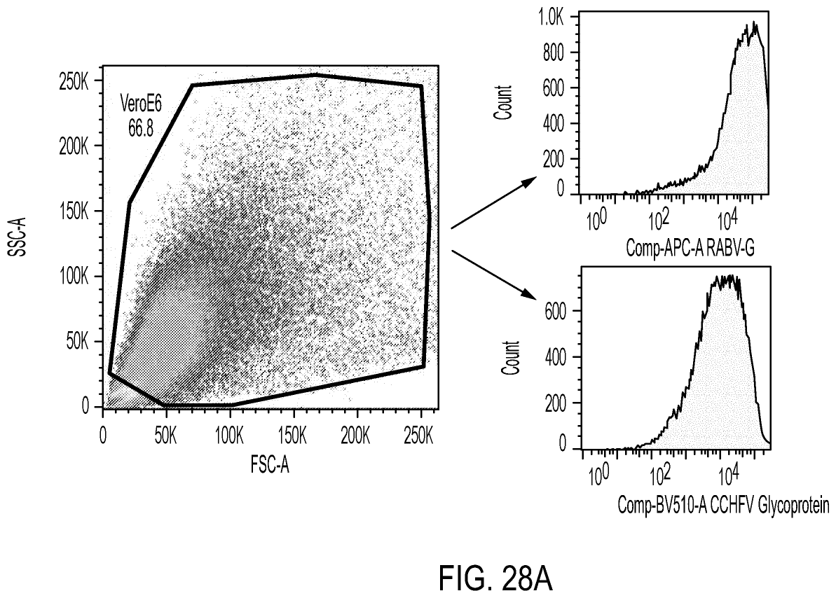

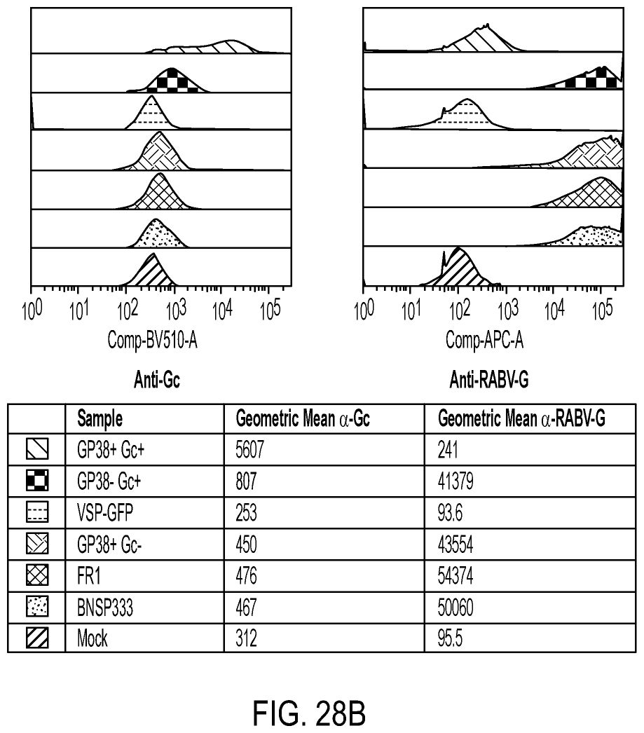

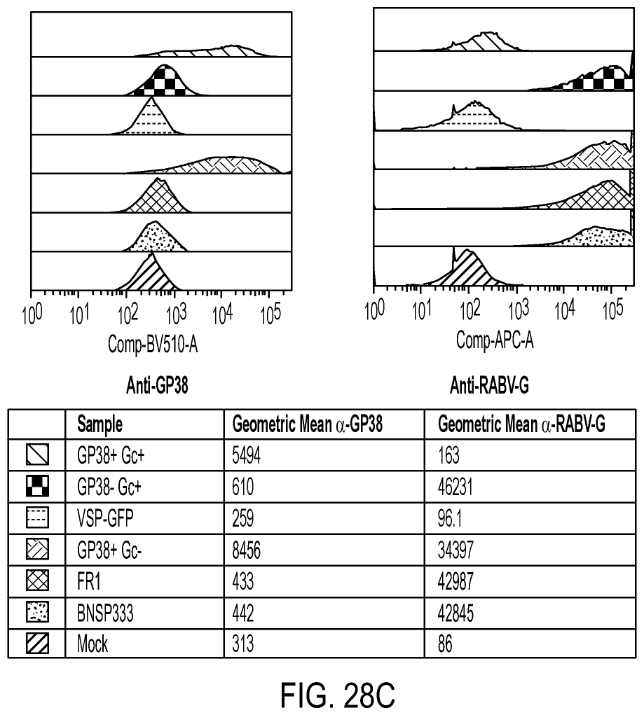

To assess the expression of the CCHFV genes in the rhabdoviral vectors, immunofluorescence (IF) surface staining and flow cytometry analysis of Vero E6 cells infected with each virus was performed. For IF, cells were infected at a multiplicity of infection (MOI) of 0.01. RABV infected cells were incubated for 72 hrs, and VSV infected cells were incubated for 24 hrs. For flow cytometry, Vero E6 cells were infected at MOI 10 for RABVs and MOI 5 for VSVs and incubated for 48 hrs or 8 hrs respectively. After infection, cells were fixed and stained with anti-RABV-G human monoclonal antibody 4C12 and either anti-CCHFV-Gc antibody 11E7 or anti-CCHFV-GP38 antibody 13G8. Surface staining of the infected cells showed that all the CCHFV proteins present in each of the rhabdoviruses were present on the cell surface (FIGS. 2A-2D and 28A-28C). RABV-G was detected from all the RABV-based vectors tested and the GP38− Gc+ virus which was engineered to contain RABV-G (FIGS. 2A-2D and 28A-28C).

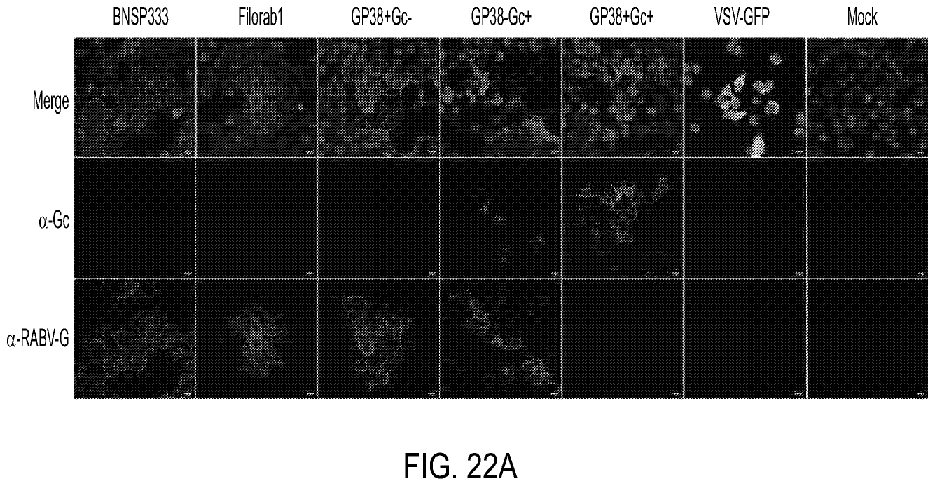

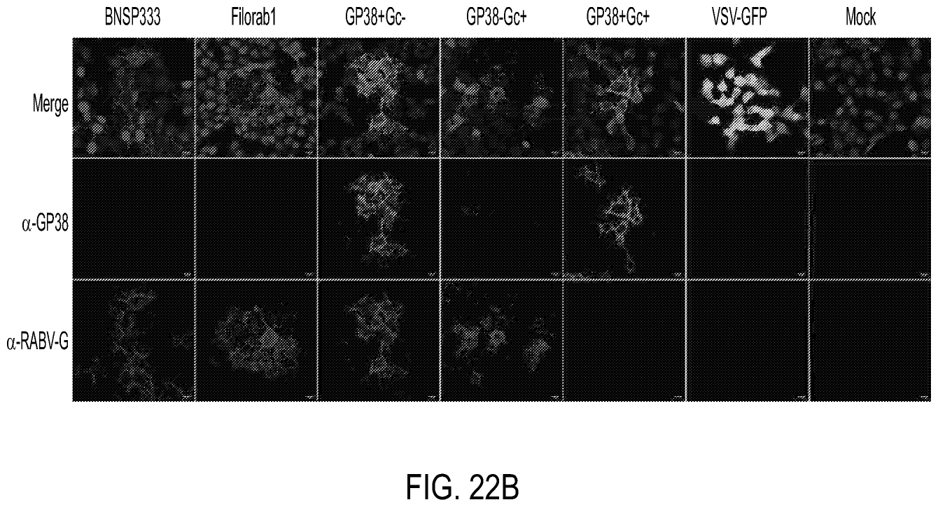

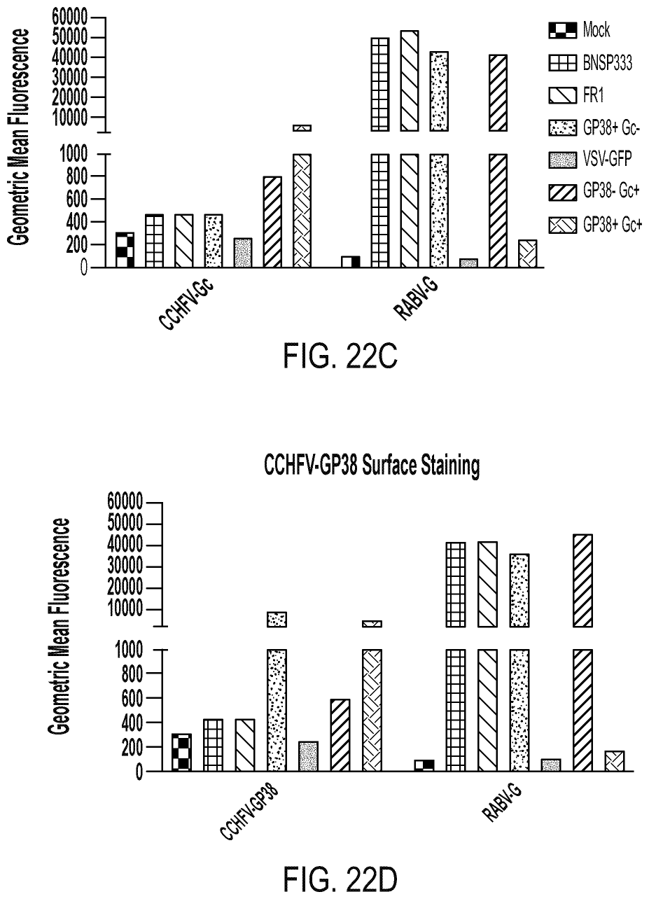

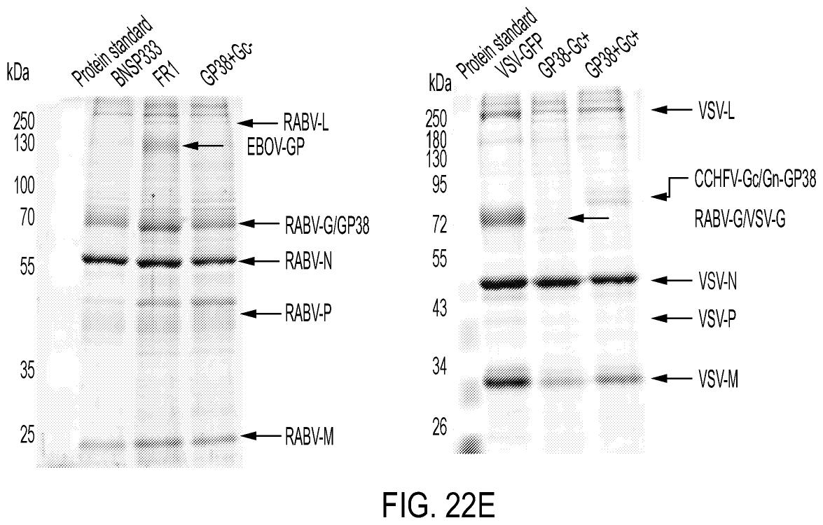

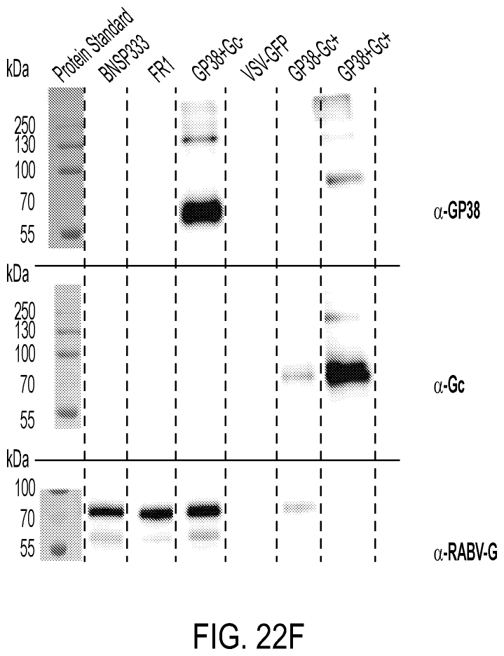

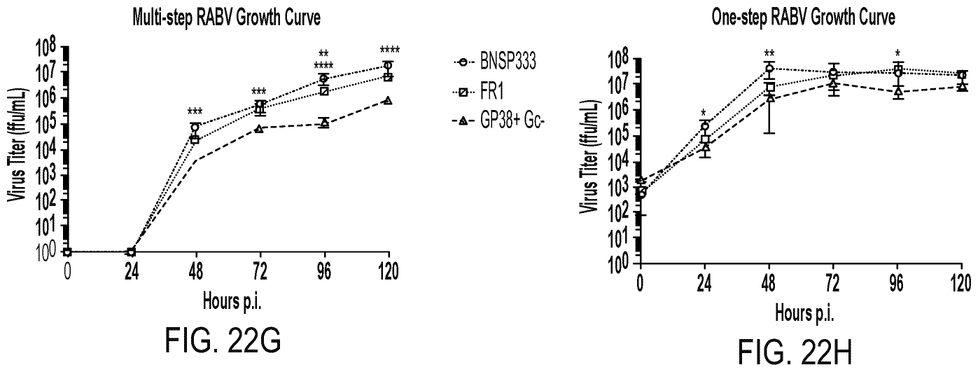

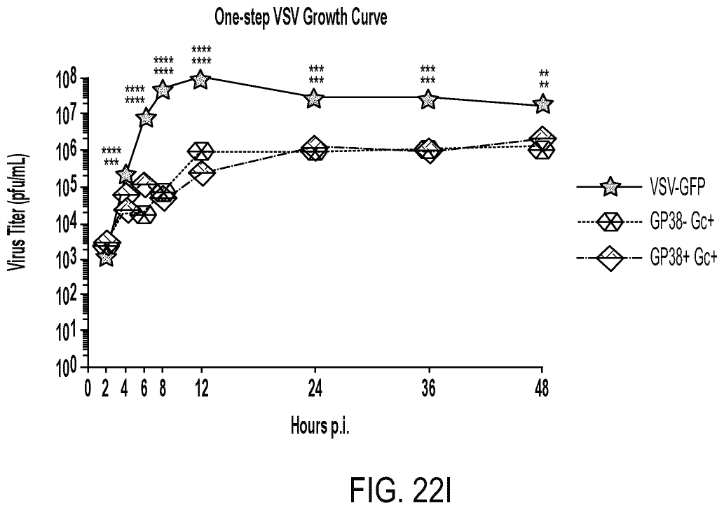

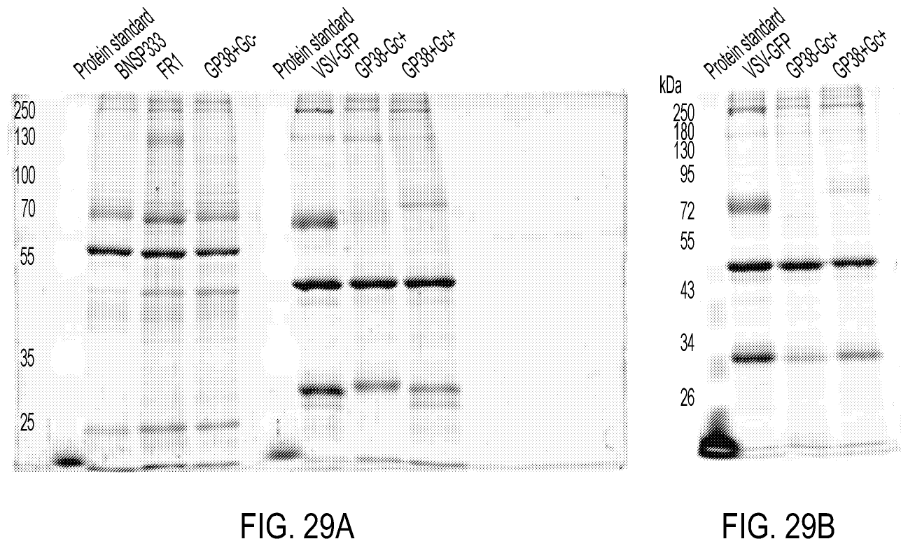

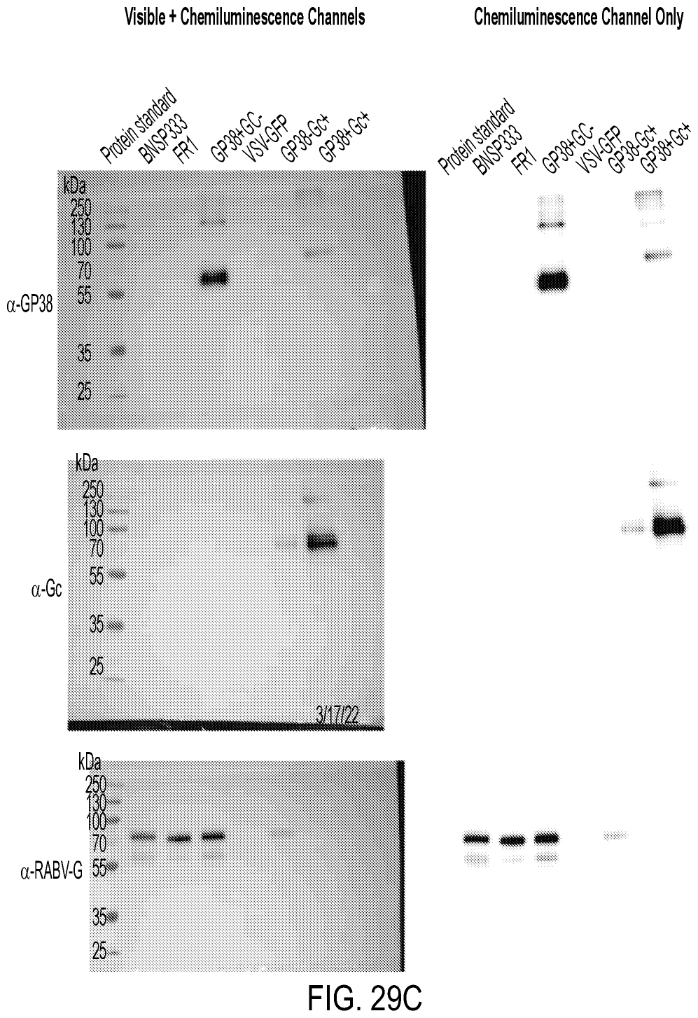

To analyze the incorporation of the glycoproteins, virions were sucrose purified and the proteins separated on SDS Page protein gels. SYPRO™ Ruby staining showed incorporation of all the native rhabdoviral proteins in each virus (FIGS. 22E and 29A-29B). Western blotting for GP38 and Gc demonstrated that only GP38+ Gc− and GP38+ Gc+ viruses incorporate GP38, whereas GP38+ Gc+ and GP38− Gc+ viruses incorporate Gc (FIGS. 22F and 29C). RABV-G was detected for the GP38− Gc+ virus (FIGS. 22F and 29C).

To analyze virus growth kinetics, multi- and one-step growth curves were performed for RABVs and one-step growth curves for VSVs. For multi-step growth curves, cells were infected at a low MOI of 0.01, and for one-step growth curves, cells were infected at a high MOI of 10. All CCHFV vaccine viruses showed slower growth kinetics compared to their parental vectors (FIGS. 22G-22I). Regardless of kinetics, all viruses grew to sufficient titers of at least 1×10⁶ focus forming units (ffu) for RABVs or plaque forming units (pfu) for VSVs.

These results show that rhabdoviruses with CCHFV glycoprotein genes are recoverable and incorporate the expected proteins into the virions.

### Example 2-3: The Mucin-Like Domain is Required for GP38 Expression

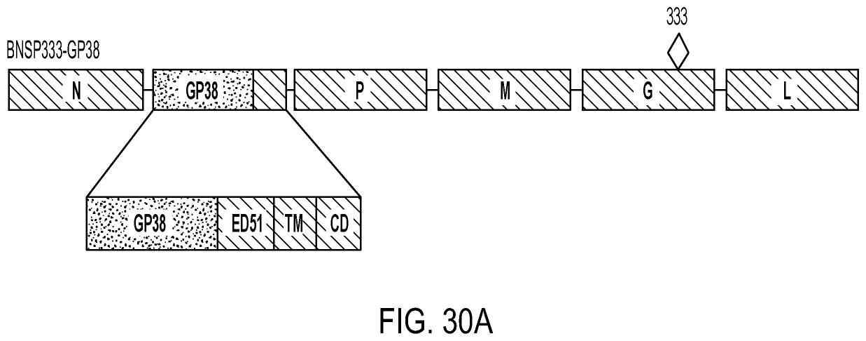

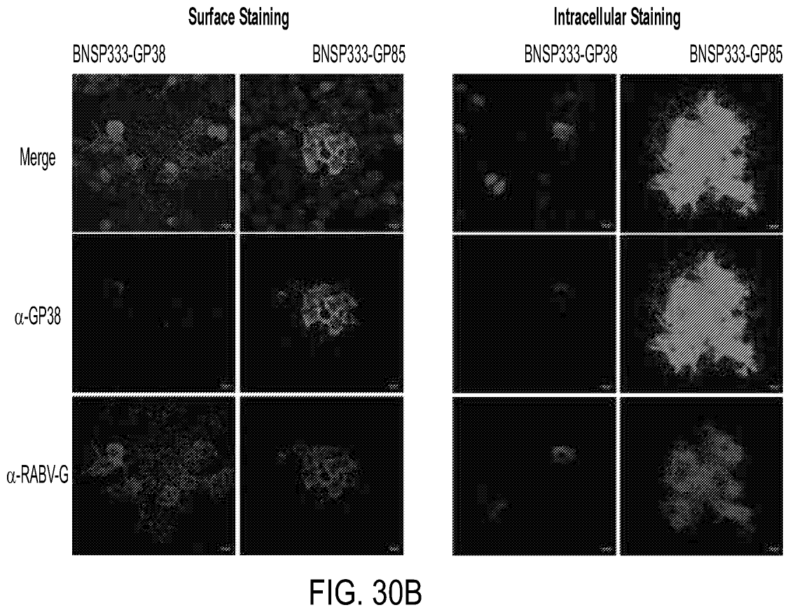

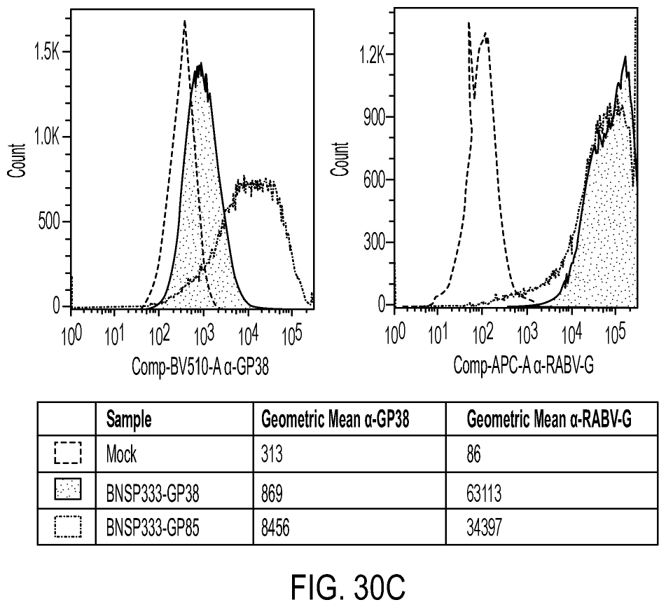

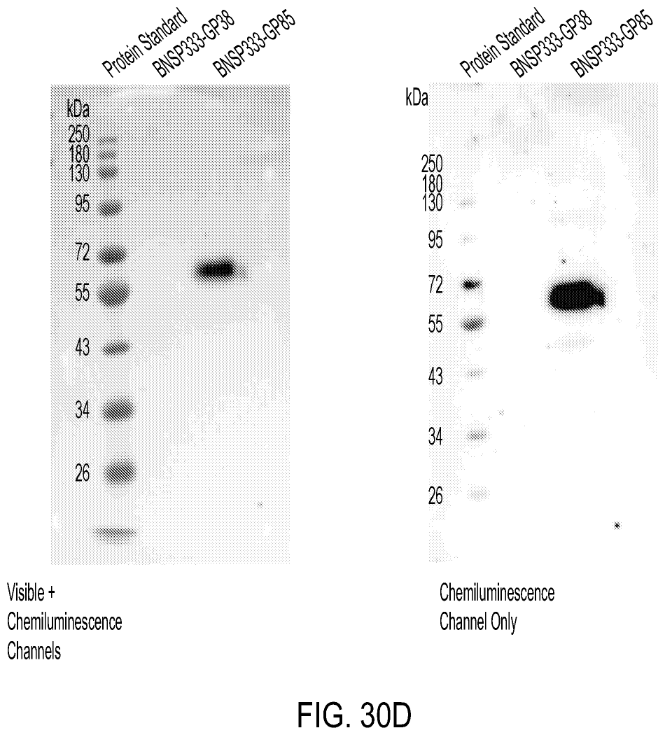

A vaccine that had GP38 with the RABV-G tail anchor but without the MLD, called BNSP333-GP38, was designed (FIG. 30A). This virus was recovered, and characterization showed very poor expression of GP38. Immunofluorescence staining for GP38 on cells infected with BNSP333-GP85 showed very strong surface and intracellular expression of GP38, while cells infected with BNSP333-GP38 showed very minimal GP38 expression (FIG. 30B). Flow cytometry analysis of cells infected with BNSP333-GP38 or BNSP333-GP85 showed comparable levels of RABV-G expression between viruses, but only BNSP333-GP85 had high levels of GP38 (FIG. 30C). Finally, western blot for GP38 of sucrose purified virions showed that BNSP333-GP38 has virtually no incorporation of GP38 into virions compared to BNSP333-GP85 (FIG. 30D). These data show that the MLD is required for proper expression and incorporation of GP38 into rhabdoviruses.

### Example 2-4: Immunogenicity of Rhabdoviral-based CCHFV Vaccines

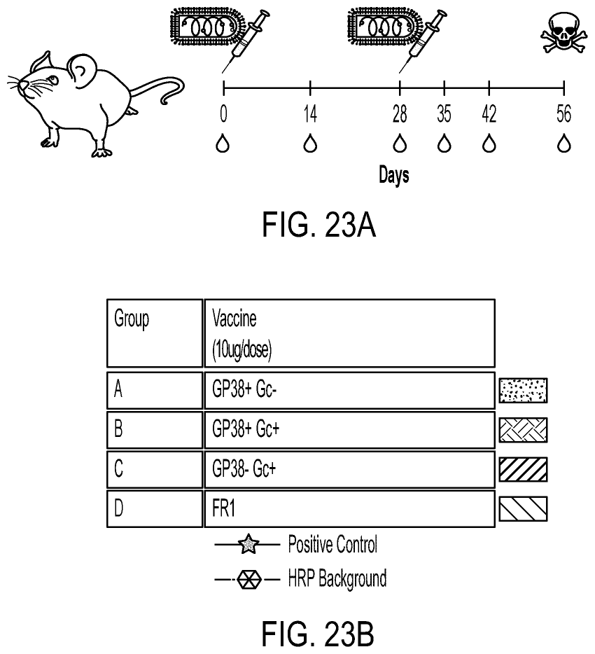

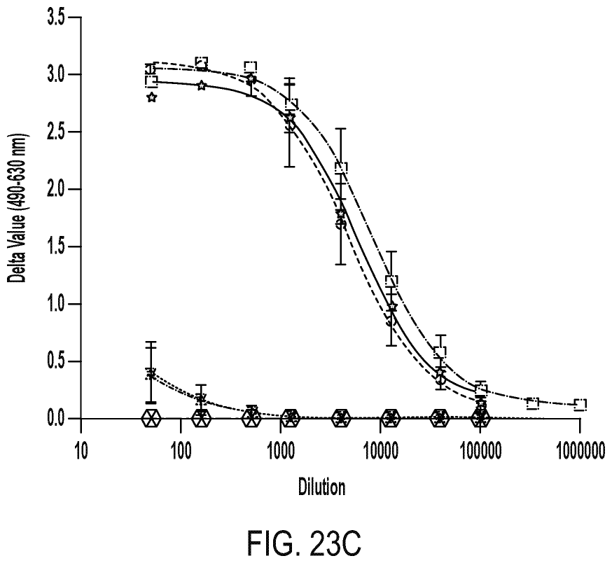

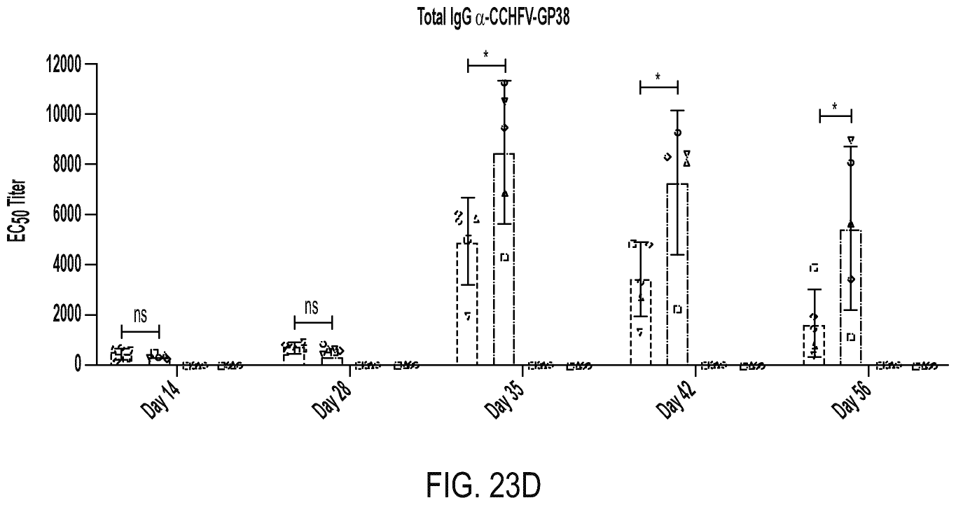

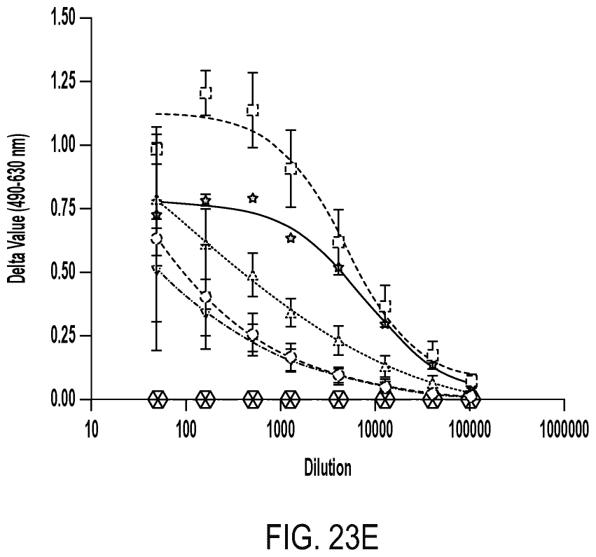

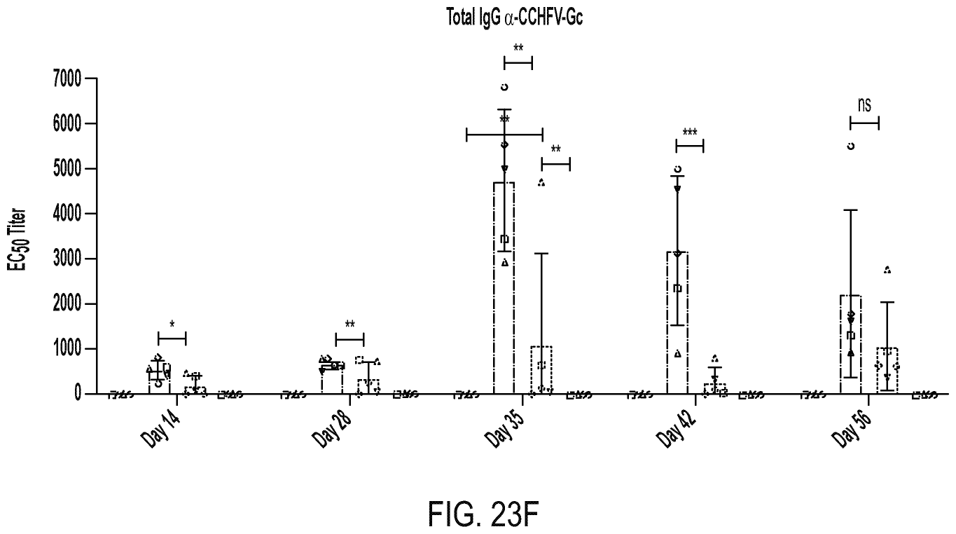

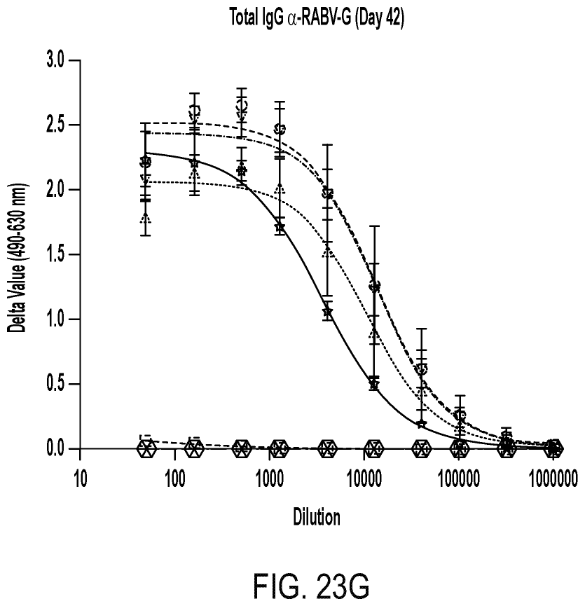

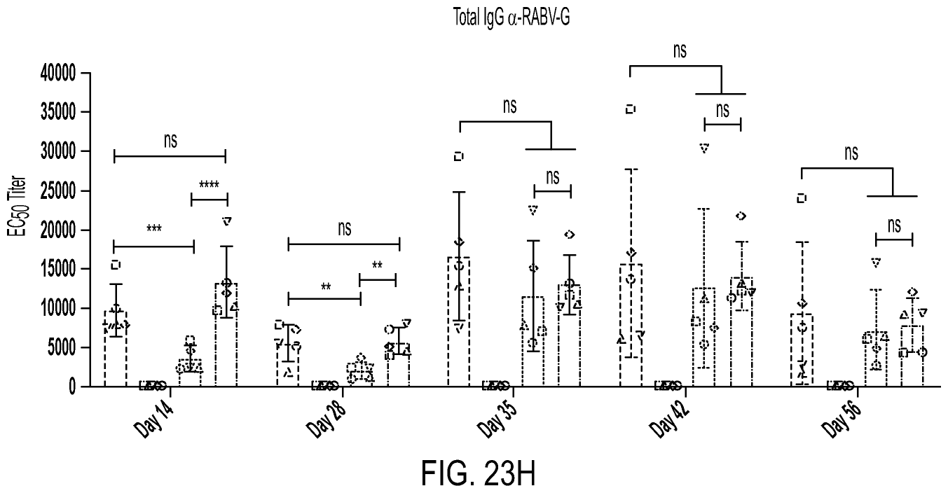

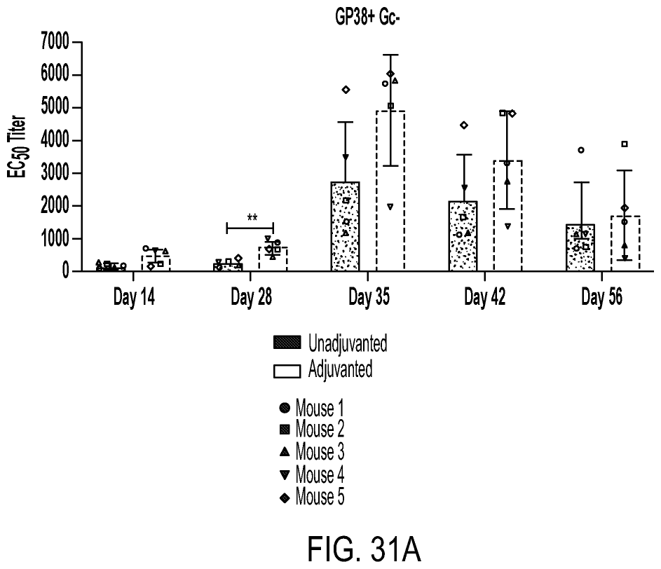

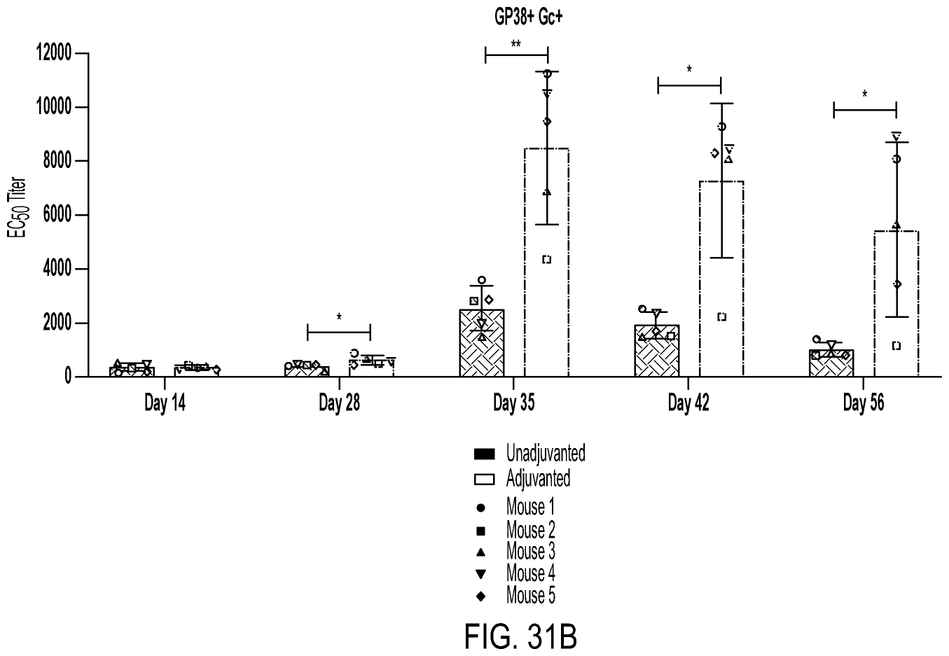

To investigate the immunogenicity of the vaccines, the present study immunized groups of 5 C57BL/6 (B6) mice with two doses, 28 days apart, of 10 μg of β-propiolactone inactivated vaccines (FIGS. 23A-23B). The present study used two groups per vaccine, one immunized with deactivated vaccine alone, the other containing deactivated vaccine adjuvanted with 5 μg of TLR-4 agonist synthetic Monophosphoryl Lipid A (MPLA), 3D (6A)-PHAD (PHAD), in a 2% squalene-in-oil emulsion (SE). The mice were bled at various time points (FIG. 23A). All mice developed antibody responses against their respective antigens by day 14 post-immunization, which increased after the boost on day 28 and were maintained out to day 56 (FIGS. 23A-23H). Using an adjuvant during vaccination typically improves the immune responses elicited by the vaccine. Adjuvanted groups showed higher antibody responses for all vaccines against their respective antigens (Shown for GP38, FIGS. 31A-31B). Thus, adjuvants were used for all subsequent studies.

### Example 2-5: Rhabdoviral-based CCHFV Vaccines Elicit a Th1-biased Antibody Response

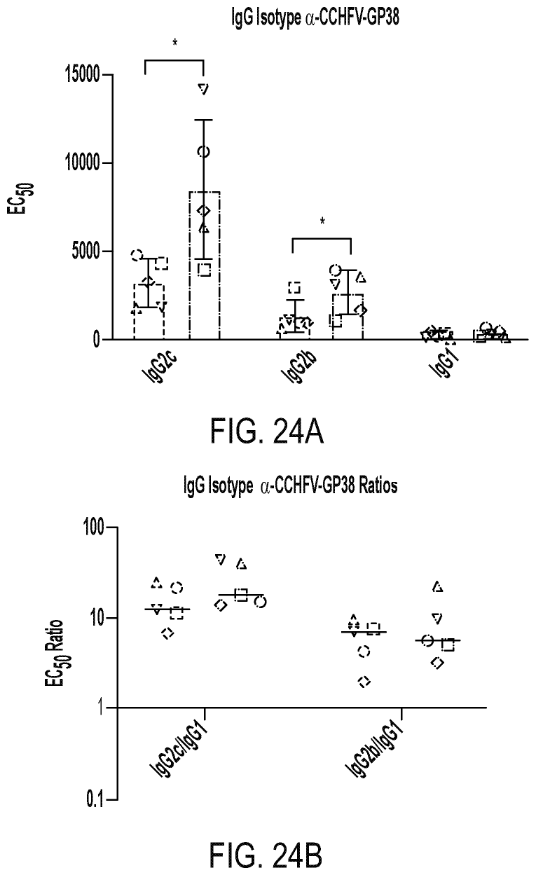

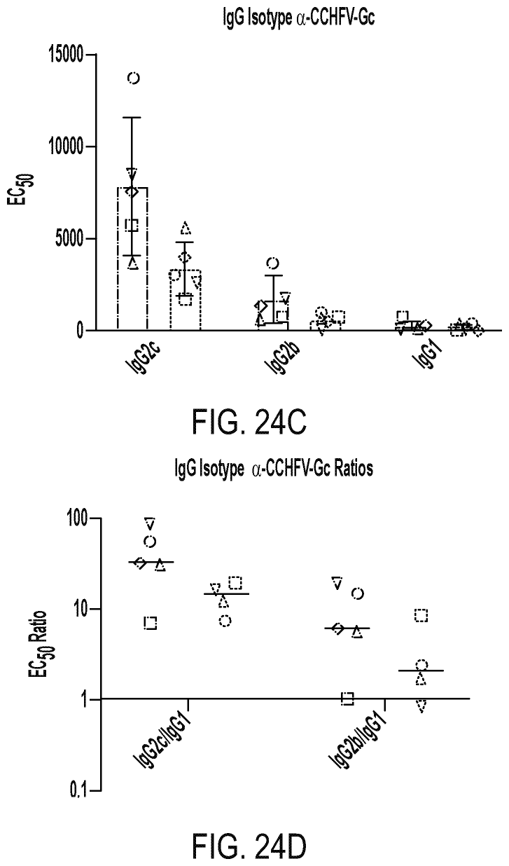

Th1 immune responses have been associated with strong anti-viral responses. In B6 mice, IgG2b and IgG2c are associated with Th1 responses, while IgG1 is associated with Th2 responses. The present study performed isotype subclass ELISAs using the day 56 sera from the immunogenicity study. All vaccines showed strong IgG2c and IgG2b antibody responses for their respective antigens, indicating a skew towards a Th1-associated response (FIGS. 24A-24D).

### Example 2-6: A VSV-based Surrogate Challenge Model as a Tool for Determining CCHFV Vaccine Efficacy

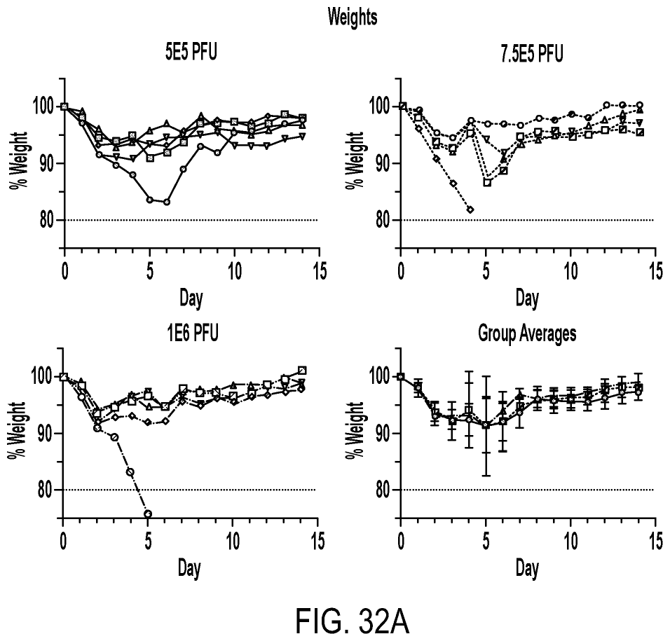

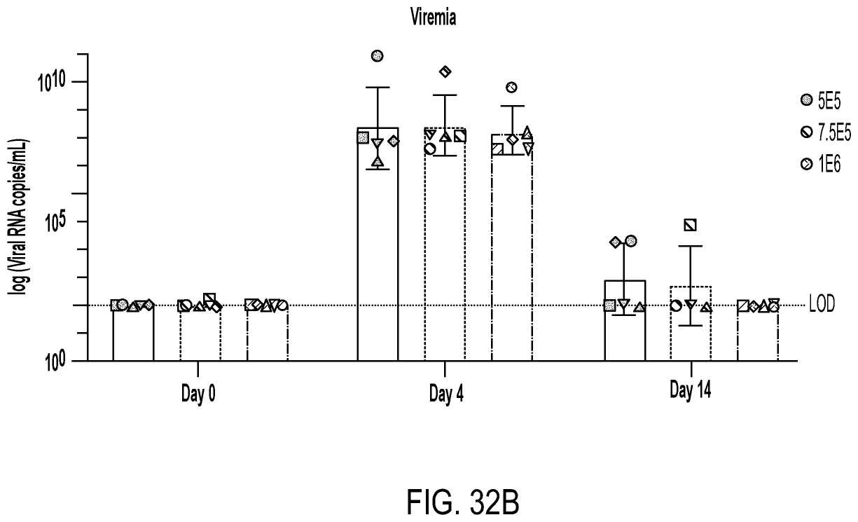

CCHFV is a BSL-4 pathogen, which makes animal experiments with CCHFV expensive. Therefore, the present study developed a VSV-based surrogate challenge model for CCHFV using the GP38+ Gc+ virus that replaces the native VSV-G with CCHFV-M. IFNAR^{-/-} mice are typically susceptible to both CCHFV and VSV, so the present study first wanted to determine the ability of the surrogate challenge virus (GP38+ Gc+) to cause disease in IFNAR^{-/-} mice. The present study challenged male mice intraperitoneally (I.P.) with either 5e5, 7.5e5 or 1e6 plaque forming units (pfu) of the GP38+ Gc+ virus, and measured weight change and viral RNA copies in the blood via qPCR as indicators of disease. Pilot studies revealed that in IFNAR^{-/-} mice, this virus consistently causes high viremia and modest weight change regardless of challenge dose but is not uniformly lethal (FIGS. 32A-32B). Thus, it was decided to use a challenge dose of 5e5pfu and use viremia as the main indicator of disease in this surrogate challenge model.

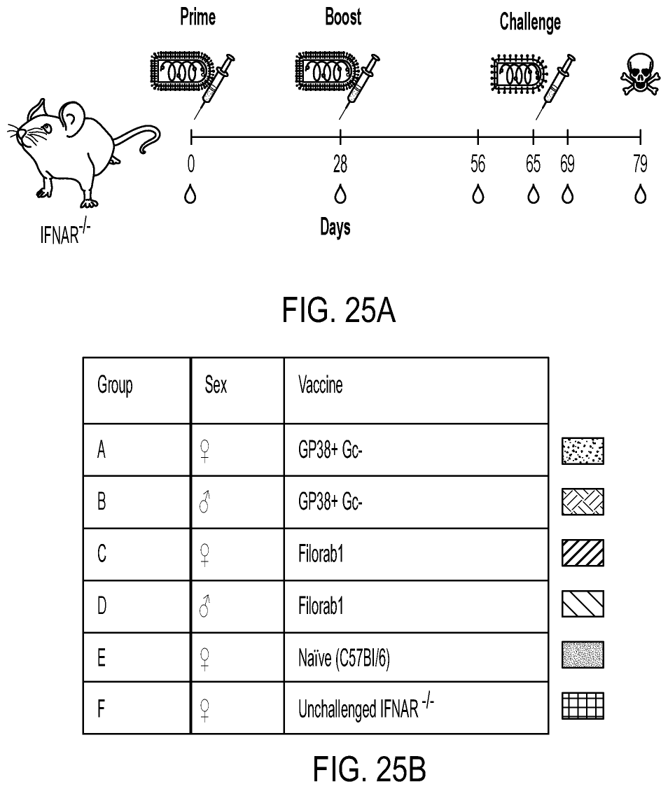

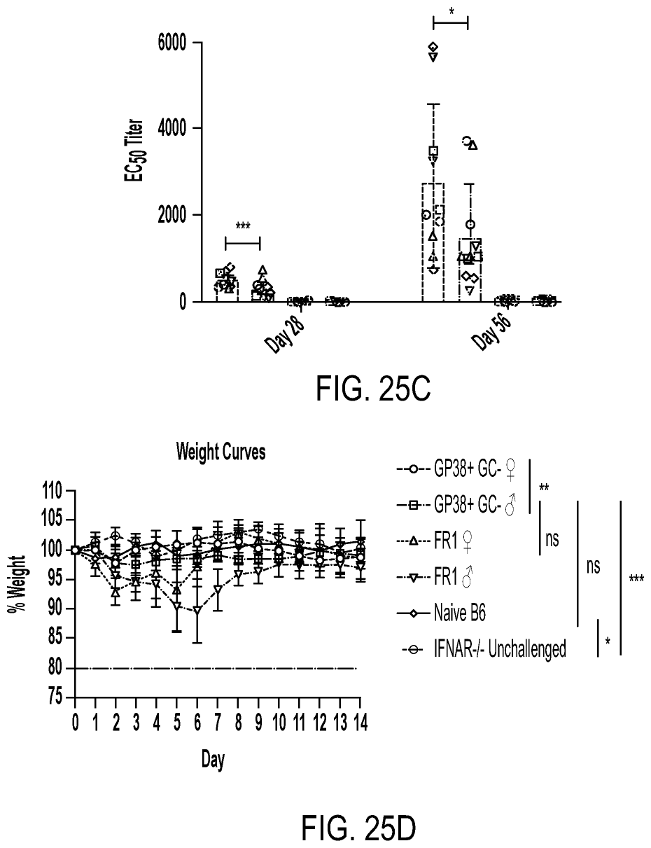

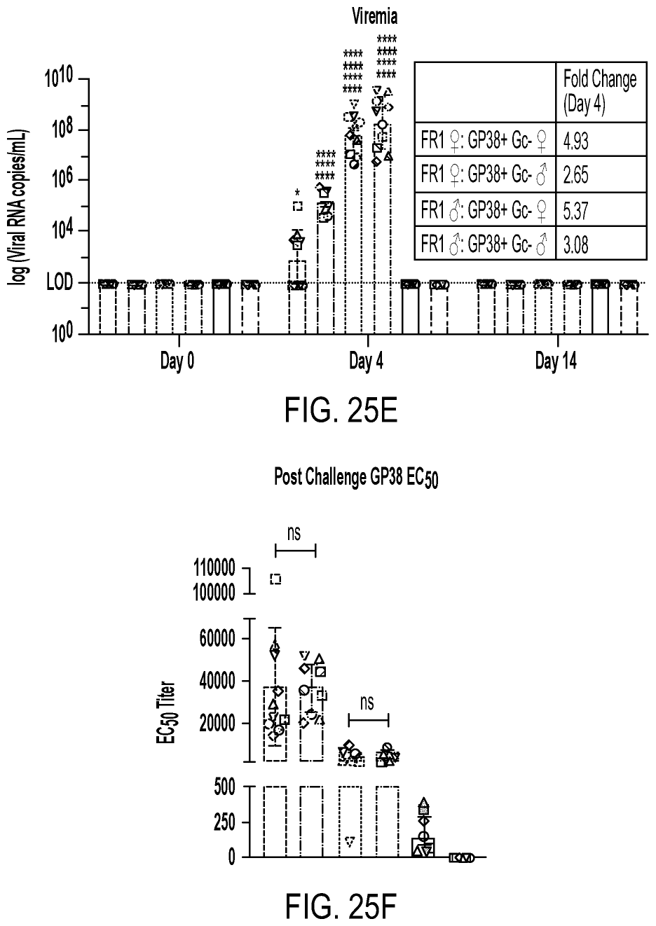

To test the utility of this challenge model for initial screening of vaccine efficacy, the present study immunized groups of male and female IFNAR^{-/-} mice with either GP38+ Gc− vaccine or control FR1 vaccine, both adjuvanted with PHAD-SE (FIGS. 25A-25B). The present study included a naïve B6 group as a control for protection since these mice are not susceptible to this virus (FIG. 25B). All IFNAR^{-/-} mice immunized with the GP38+ Gc− vaccine developed antibodies against CCHFV GP38, but gender differences in antibody titer were observed (FIG. 25C).

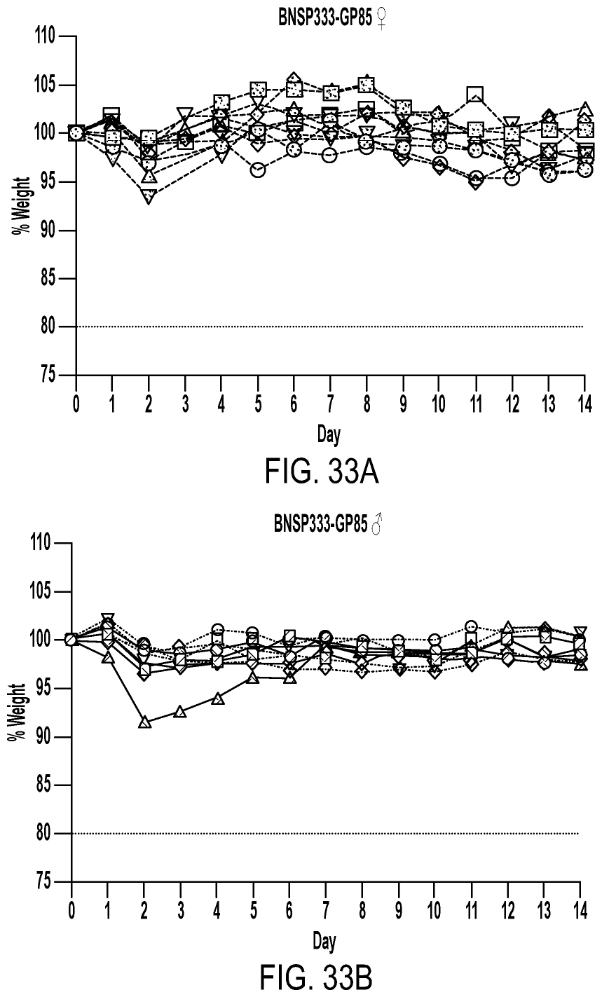

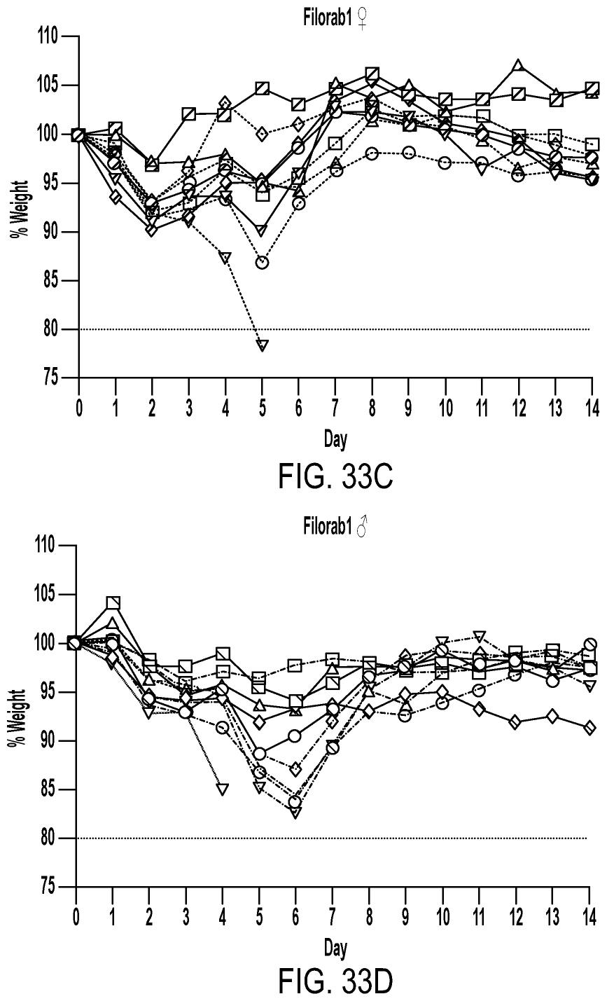

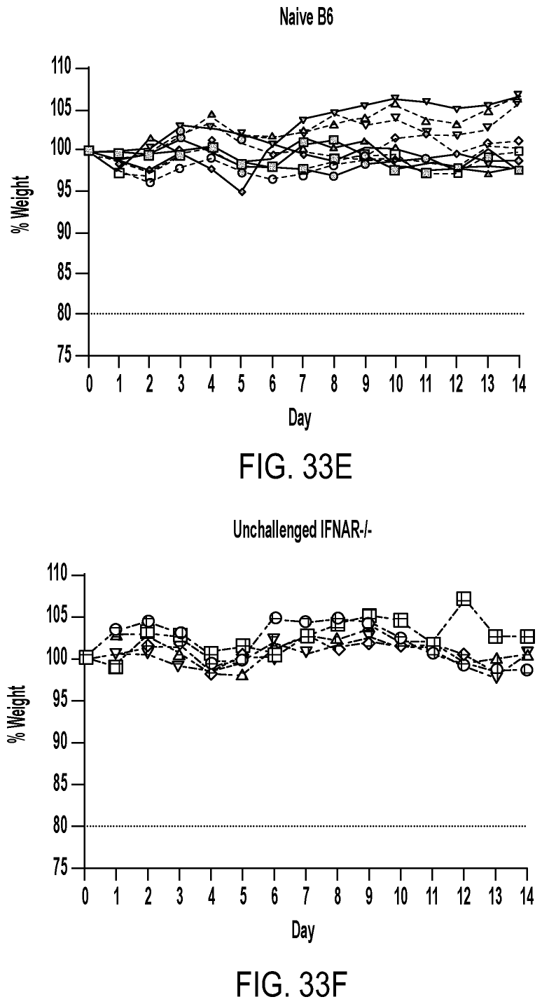

On day 65 post immunization, the vaccinated IFNAR^{-/-} and naïve B6 mice were challenged I.P. with 5E5pfu of the surrogate challenge virus (GP38+ Gc+). Mice immunized with the GP38+ Gc− vaccine showed minimal weight fluctuation post-challenge, while mice immunized with the FR1 vaccine showed modest weight loss (FIGS. 25D and 33A-33F). One female and one male mouse from the FR1 immunized groups met endpoint euthanasia criteria on day 5 post-challenge. Mice vaccinated with the FR1 vaccine showed high viral RNA copies in the blood at 4 days post-infection, which were 3-5-fold higher compared to mice immunized with the GP38+ Gc− vaccine, with some females completely clearing the virus (FIG. 25E). Mice vaccinated with the GP38+ Gc− had a boost in GP38-specific antibody titers post-challenge (FIG. 25F).

These data show that the VSV-based surrogate challenge model for CCHFV can be used to test vaccine efficacy under BSL-2 conditions.

### Example 2-7: Rhabdoviral-based CCHFV Vaccine Efficacy Against Wildtype CCHFV Challenge

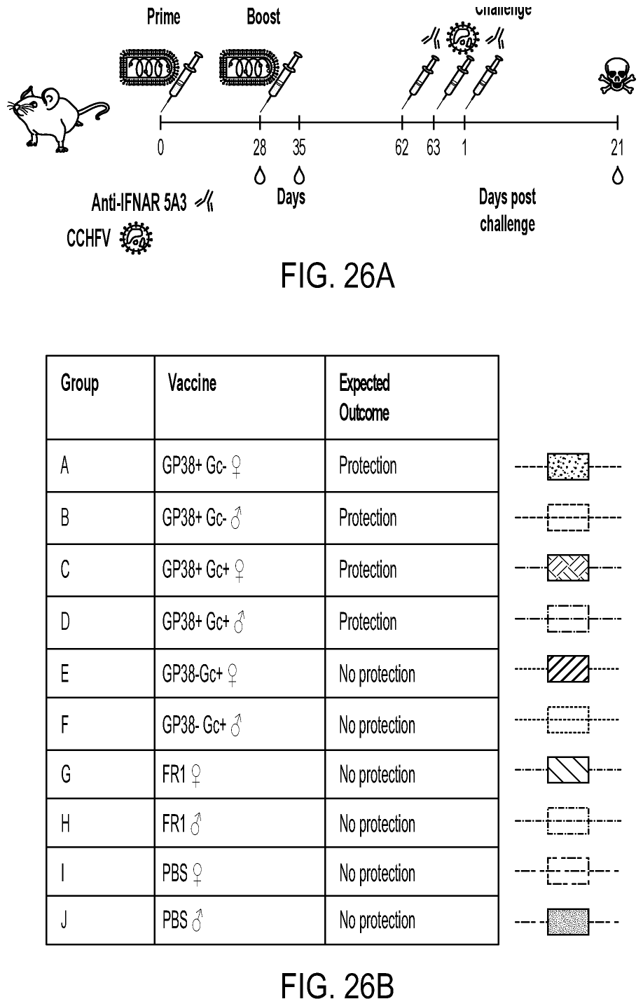

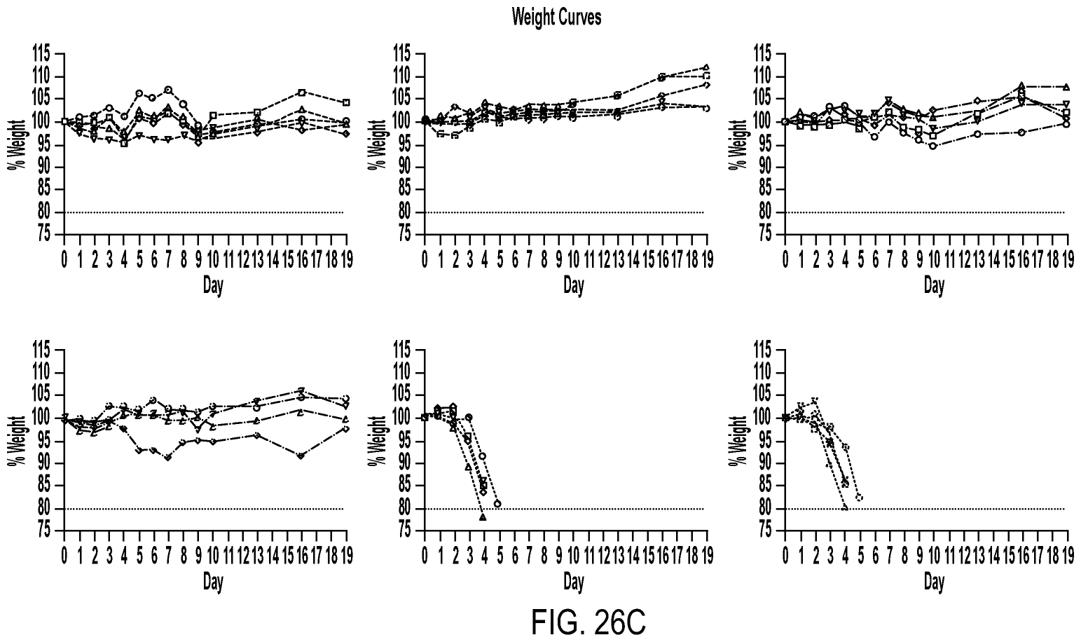

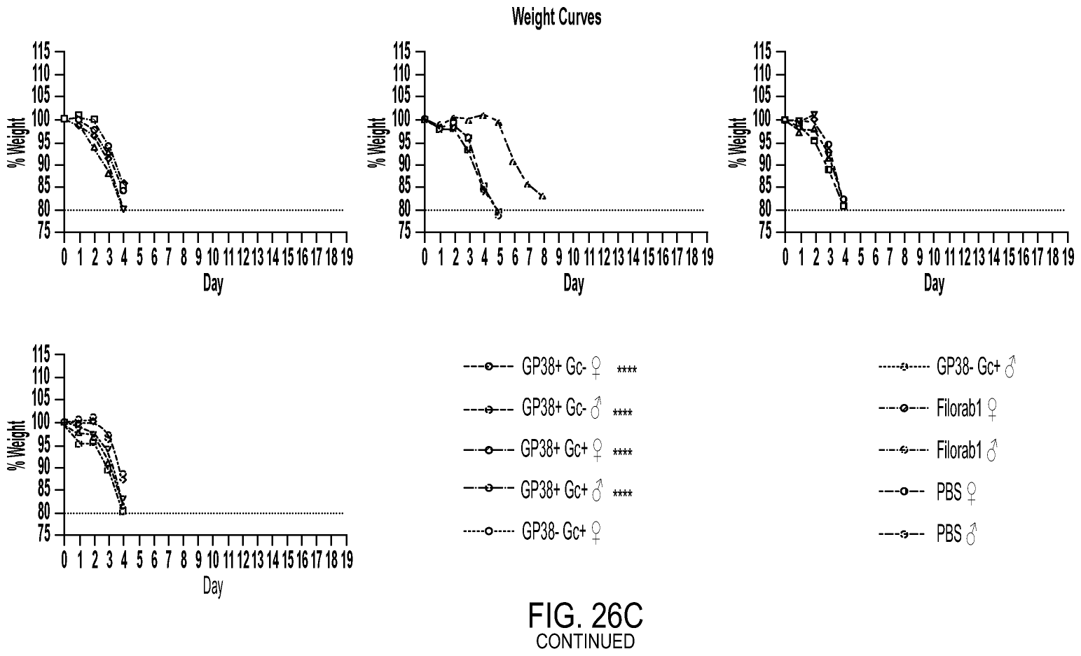

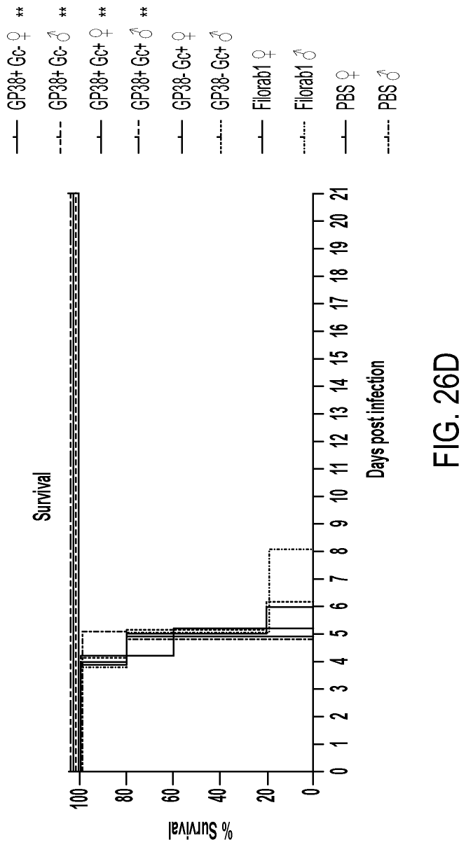

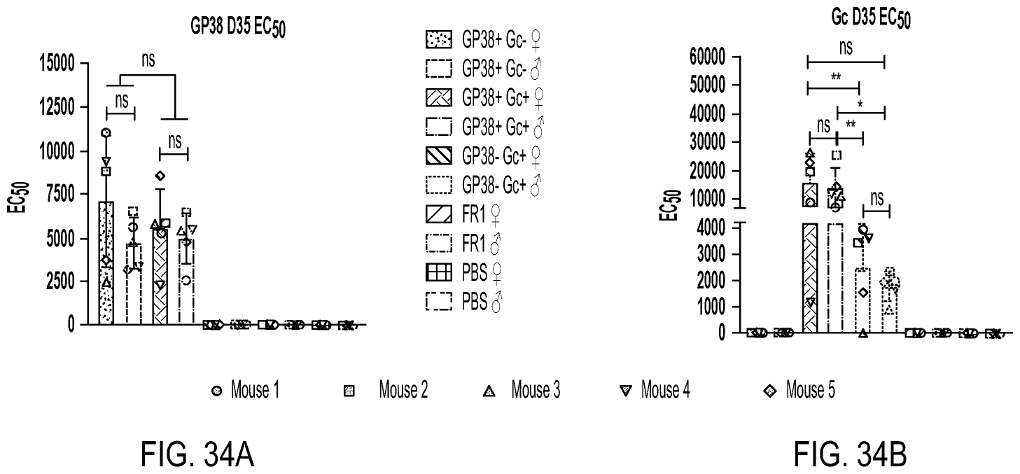

To determine the protective efficacy of these rhabdoviral-based CCHFV vaccines, a challenge experiment with wildtype (WT) CCHFV was performed. B6 mice were immunized with 10 μg of vaccine/dose adjuvanted with PHAD-SE following the same prime/boost schedule used above for the immunogenicity studies (FIG. 26A). For this study, two groups of 5 mice per vaccine were utilized, one female and the other male, to detect any differences between the sexes. ELISAs against GP38 and Gc with sera collected at day 35 showed that all vaccines elicited strong antibody responses against the expressed CCHFV antigens, and there were no differences in antibody titers between sexes in the B6 mice (FIGS. 34A-34B).

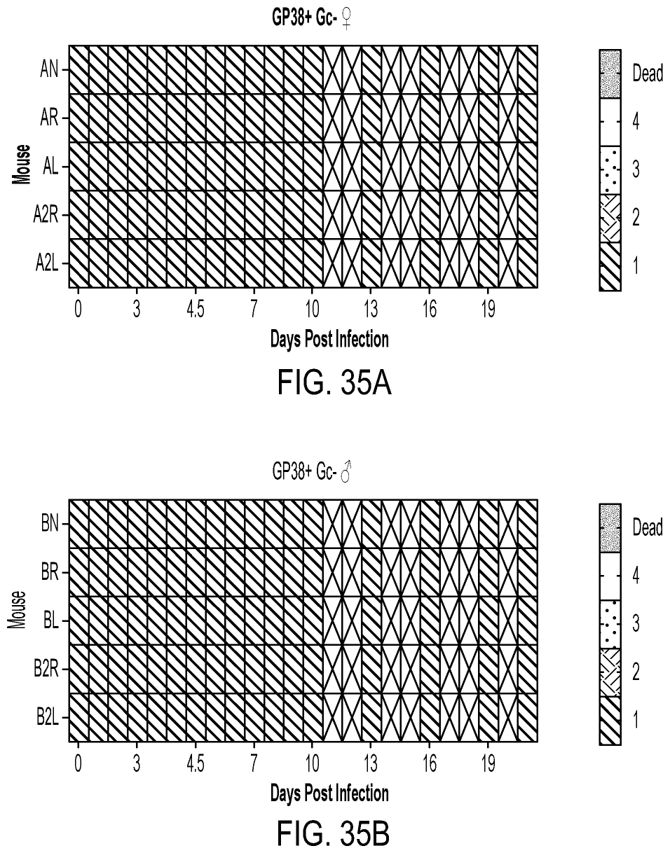

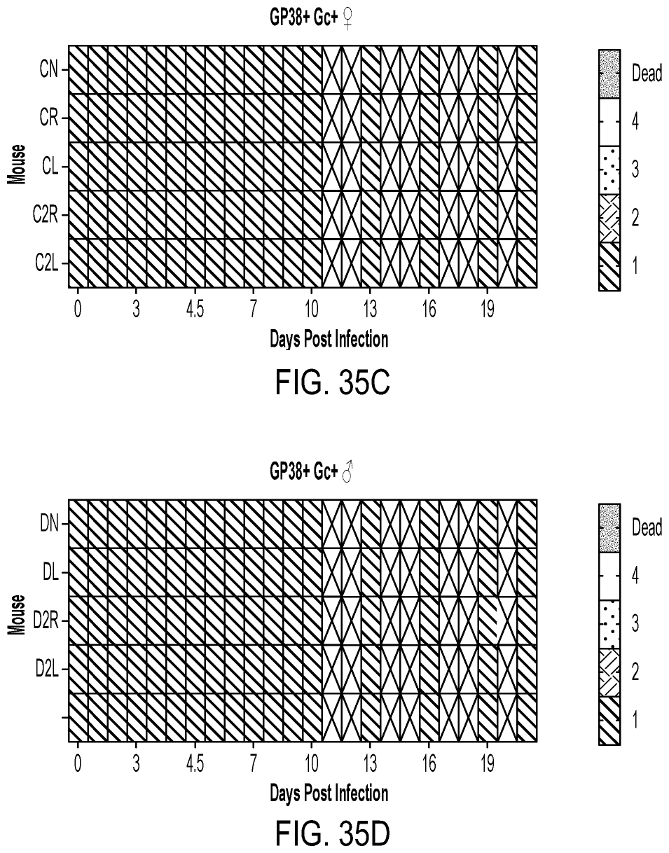

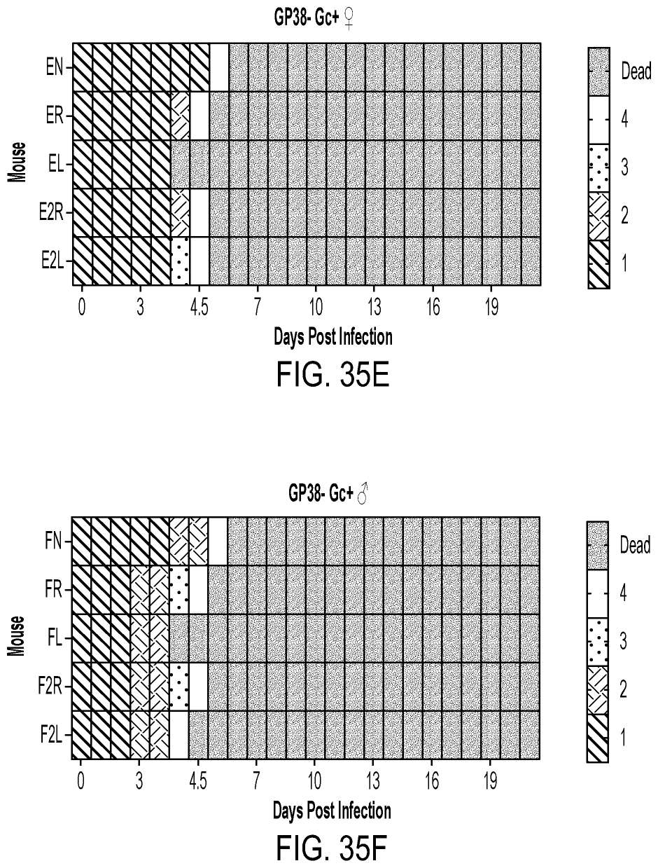

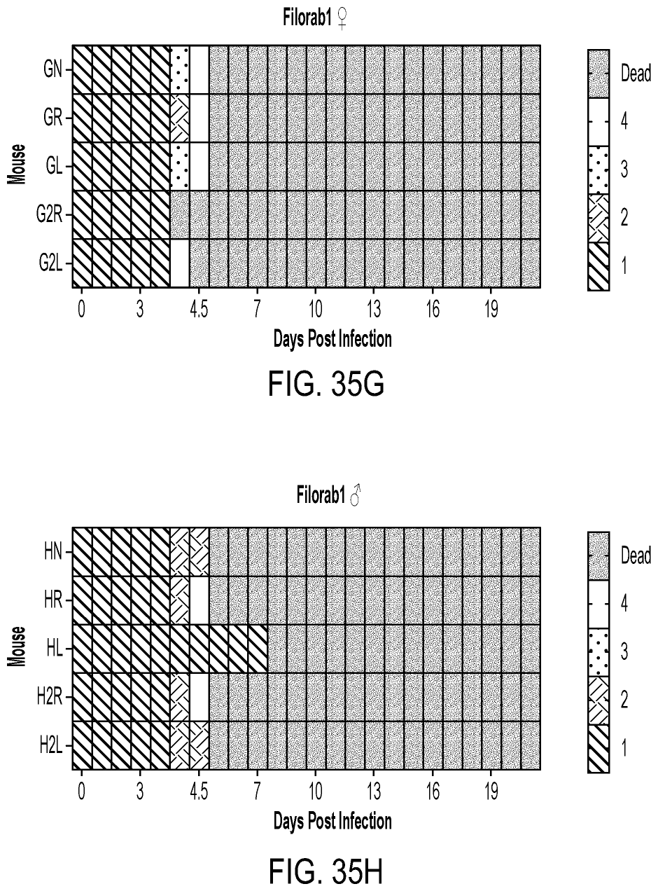

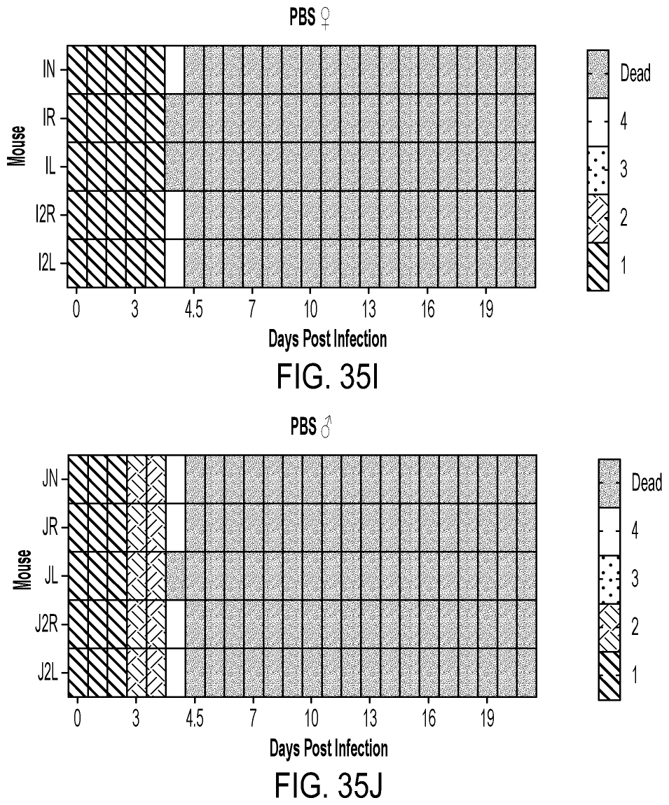

Given that WT mice are resistant to CCHFV infection, the immunized B6 mice were treated with anti-IFNAR monoclonal antibody mAb-5A3 to make them susceptible and then challenged I.P. with 1000pfu of CCHFV, strain IbAr10200. Mice vaccinated with either the GP38+ Gc− or GP38+ Gc+ vaccines maintained weight throughout the course of the challenge, while mice vaccinated with GP38− Gc+, FR1, or PBS showed dramatic weight loss starting by day 3 post challenge (FIG. 26C). All mice vaccinated with either GP38+ Gc− or GP38+ Gc+ vaccines survived challenge out to day 21 and did not show any outward clinical signs of disease (FIGS. 26D and 35A-35J). However, all mice vaccinated with either GP38− Gc+, FR1 or PBS succumbed to disease, with most mice reaching endpoint euthanasia criteria between days 4-6, except for one male mouse vaccinated with FR1 (FIG. 26D). There were no significant differences in weight loss or survival between mice of different sexes immunized with the same vaccine.

These results confirmed that only mice receiving vaccines containing GP38 (i.e., GP38+Gc− and GP38+ Gc+) were protected against lethal CCHFV challenge.

### Example 2-8: Vaccine-Induced Virus Neutralization Does Not Correlate with Protection

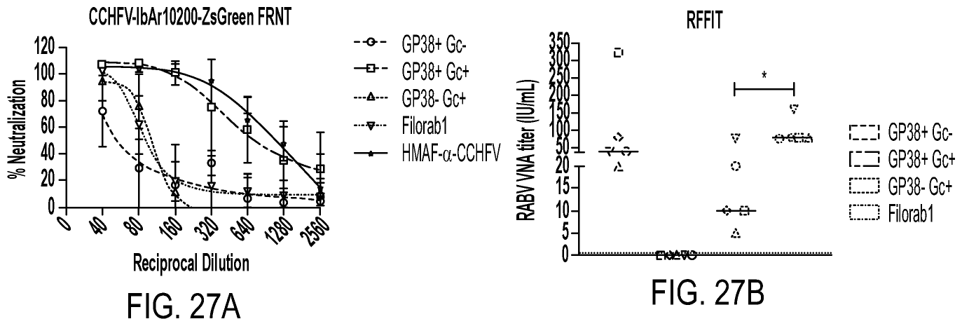

To determine the CCHFV neutralizing capabilities of the rhabdoviral-based CCHFV vaccines, a focus reduction neutralization test (FRNT) was performed using a recombinant CCHFV expressing ZsGreen. Previous studies have suggested that protection from lethal challenge is achieved with neutralizing antibody titers of 1:160, which in this assay, corresponds to 100% virus neutralization when using the hyper-immune mouse ascitic fluid (HMAF) control. The GP38+ Gc+ vaccine had a FRNT50 of <1:1280 and showed neutralizing activity comparable to HMAF, with 100% virus neutralization at a 1:160 serum dilution (FIG. 27A). The GP38+ Gc− and GP38− Gc+ vaccines demonstrated minimal neutralization at a 1:160 serum dilution, similar to FR1 immunized control mice (FIG. 27A). These data indicate that vaccine-induced neutralizing antibodies are not the mechanism of protection for these vaccines.

The present study also analyzed a virus neutralization assay (VNA) for RABV. For RABV, induction of high levels of neutralizing antibodies post-vaccination correlates with protection. As measured through the rapid fluorescent focus inhibition assay (RFFIT), mice immunized with GP38+ Gc−, GP38− Gc+, or FR1 vaccines all showed high levels of RABV neutralizing antibodies, well above the 0.5 international units (IU)/mL threshold considered protective by the WHO (FIG. 27B). No RABV-neutralization was observed in mice immunized with the GP38+ Gc+ vaccine (FIG. 27B).

### Example 2-9

CCHFV is an emerging disease for which no licensed treatments or vaccines are available. To this end, the present study developed an inactivated RABV-vectored CCHFV vaccine targeting the GP38 protein. This killed virus vaccine platform was safe to administer to both WT and immunocompromised (IFNAR−/−) mice and showed protection against lethal challenges in mice. Although GP38 is unique to the nairoviruses, it has not been widely investigated as a potential vaccine target. GP38 is indispensable for viral replication and GP38 targeted immune responses elicited protection against CCHFV challenge. Thus, it was decided to tailor the vaccine approach to target GP38.

The present study initially constructed a recombinant RABV containing a chimeric GP38/RABV G gene. This virus had poor expression and no GP38 incorporation, indicating that the MLD is required for GP38 processing. There is some evidence in the literature supporting this idea. Deleting of the MLD changes GP38 localization and affects the incorporation of the structural glycoproteins into tc-VLPs. However, it is believed that the present study has shown here for the first time with a live viral vector that the MLD is required for the proper processing of CCHFV GP38. It is believed that this is likely the reason that the present study observed better protection than the DNA vaccine targeting GP38 alone. The GP38 DNA vaccine did not contain the MLD and thus GP38 was not sufficiently processed and unable to elicit the necessary immune responses for protection.

Moreover, the present study developed a BSL-2 surrogate challenge model to test CCHFV vaccine efficacy, given the challenges of performing such studies in BSL-4 labs. Such a model using a VSV with its native glycoproteins replaced with the LASV glycoproteins was useful for determining the mechanism of protection for a RABV-based LASV vaccine. While the CCHFV model was not uniformly lethal in IFNAR^{-/-} mice, it did cause consistently high levels of viremia, an indicator of significant replication in the host. Additionally, the present study saw that the GP38+ Gc− vaccine elicited protection in this surrogate challenge model, demonstrating its utility in analyzing vaccine protective efficacy. Of note, the results detected in the surrogate model translated well to the finding in the WT CCHFV challenge further indicating the model's usefulness.

It was hypothesized that only CCHFV-M targeting vaccines containing GP38 would be protective against CCHFV challenge. The present study confirmed the hypothesis that GP38 is required and sufficient for protection. The present study saw that both the GP38+ Gc− and GP38+ Gc+ vaccines protected 100% of mice against lethal CCHFV challenge, while our control mice, including the GP38− Gc+ vaccinated mice, all succumbed to challenge. The full-length CCHFV-M gene or individual components have been tested as a CCHFV vaccine target in many vaccine strategies. In line with the hypothesis that GP38 is required for protection when targeting CCHFV-M, vaccine strategies that use the entire M gene, such as DNA vaccines or live viral vectors, have shown protection against WT CCHFV challenge. The study by Appelberg et al. (2021) indicated that their mRNA vaccine targeting the CCHFV glycoproteins only included Gc and GN, and this vaccine was shown to be protective. However, the sequence of this vaccine encodes for the full CCHFV-M gene, meaning GP38 was included in this vaccine (personal correspondence D. Weissman, 2022). Conversely, those vaccine strategies that exclude GP38 or do not develop immune responses against GP38 are not protective against CCHFV challenge. Thus, the present study confirmed that GP38 is an excellent vaccine target for CCHFV.

The GP38+ Gc− vaccine was protective against WT CCHFV challenge, with no visible weight loss or clinical signs. These results are comparable to other vaccine strategies targeting CCHFV-M that were protective against CCHFV challenge, including a live VSV-vectored vaccine, live MVA-vectored vaccine, CCHFV-M DNA vaccine and CCHFV-M mRNA vaccine. However, the present vaccine candidate has a few advantages over these other strategies. As mentioned above, the present vaccine is a deactivated virus, making it safe to administer to various immunocompromised populations and pregnant women, unlike live virus vaccines. The DNA vaccine used a three dose immunization schedule, while the present vaccine showed protection after only two doses. Major drawbacks of the mRNA platform are waning immunity and the necessity to store these vaccines at extremely cold temperatures. In contrast, the RABV vaccine has been shown to induce life-long immunity in humans and can be vaporized and remain stable at various temperatures, including storage at 50° C. for up to 2 weeks. Additionally, the means of production for RABV-based vaccines already exists given that this vaccine has been produced and used for decades.

Mice immunized with our various CCHFV vaccines showed strong antibody responses against their respective CCHFV glycoproteins and RABV-G with a skew towards a Th1 response. Two different CCHFV DNA vaccination strategies have investigated the types of antibody responses elicited from vaccination and showed that Th1 biased antibody responses were protective against CCHFV challenge. Additionally, one of the studies demonstrated that vaccines eliciting a Th2 biased response were less protective compared to those eliciting a Th1 biased response. The results of our vaccine study agree with these studies, further indicating that Th1 associated responses elicited by CCHFV vaccines are important for protection.

The present study study indicates the present vaccine is effective in mice regardless of sex or immune status, something that is very important for an ideal vaccine candidate.

In summary, this study shows that for CCHFV-M vaccines, GP38 is required and sufficient for protection. The GP38+ Gc−(BNSP333-GP85) vaccine can progress to further testing in NHP and is an excellent candidate to be moved to the clinic.

### Examples 2-10: Materials and Methods

### Animals

C57BL/6 mice (Charles River) and B6.129S2-Ifnar1^{tm1Agt}/Mmjax (The Jackson Laboratory) mice ages 6-10 weeks were used in this study. Both males and females were used. Mice used in this study were handled in adherence to the recommendations described in the Guide for the Care and Use of Laboratory Animals and the guidelines of the National Institutes of Health, the Office of Animal Welfare, and the United States Department of Agriculture. All animal protocols were approved by the Institutional Animal Care and Use Committee (IACUC) of Thomas Jefferson University (TJU) or University of Texas Medical Branch (UTMB) for experiments performed at each facility. The facilities where this research was conducted are fully accredited by the Association for Assessment and Accreditation of Laboratory Animal Care International. Mice were housed in cages, in groups of 5, under controlled conditions of humidity, temperature, and light (12 hr light/12 hr dark cycles). Food and water were available ad libitum. Animal procedures at TJU were conducted under 3% isoflurane/O2 gas anesthesia by trained personnel under supervision of veterinary staff.

### Cells

Vero (ATCC® E6™), 293T (available from the Schnell laboratory), BSR (available from the Schnell laboratory) and BEAS-2B (ATCC® CRL-9609™) cells were cultured using DMEM (Corning®) with 5% fetal bovine serum (FBS) (Atlanta-Biologicals®) and 1% Penicillin-Streptomycin (P/S) (Gibco®). 293F (ATCC® CRL-12585™) cells were cultured using FreeStyle™ 293 Expression Medium (Gibco®) with 2X Glutamax (Gibco®). Mouse neuroblastoma (NA) (available from the Schnell laboratory) cells were cultured using RPMI (Corning®) with 5% FBS and 1X P/S. Human hepatocarcinoma cells (HuH-7) (available from the Bente Laboratory) and SW-13 cells (available from the Bente Laboratory) were maintained in Dulbecco's modified Eagle's medium (DMEM) supplemented with 10% FBS (Invitrogen, Carlsbad, CA), 2 mM L-glutamine (Invitrogen), and 1% P/S (Invitrogen), cumulatively called D10. All cells except 293F were stored in incubators with 5% CO₂ at 37° C. for normal cell culture or 34° C. for virus infected cells. 293F cells were stored in incubators with 8% CO₂ at 37° C. and shaking at 140 rpm.

### Viruses

RABV strain CVS-11 was produced in our laboratory on NA cells and is available upon request. A recombinant CCHFV, strain IbAr10200, ZsGreen reporter virus expressing the fluorescence tag on the N-terminus of the genomic S-segment ORF, designated rCCHFV-ZsGreen, was used for the fluorescence reduction neutralization test (kindly provided by Dr. Éric Bergeron of Centers for Disease Control and Prevention, Atlanta, GA). CCHFV strain IbAr10200 was obtained from the World Reference Collection of Emerging Viruses and Arboviruses at UTMB (WRCEVA, passaged 13 times in suckling mice and one time in Vero E6; Genbank sequences: NC005302, NC005300, and NC005301) and was passaged twice in SW-13 cells (ATCC, CCL-105) before use. All work with CCHFV was performed in a biosafety level 4 facility at the Galveston National Laboratory, University of Texas Medical Branch, Galveston, TX, in accordance with the approved Institutional Biosafety Committee protocols.

Generation of Rhabdoviral Vaccine Vector eDNA

The human codon-optimized CCHFV-M, IbAr 10200 strain (CCHFV-coM) (Garrison et al. PLOS Neglected Tropical Diseases 11, e0005908-e0005908 (2017)), used to develop the CCHFV vaccines was a generous gift from Dr. Aura Garrison (USAMRIID, Frederick, MD). All BNSP333 (McGettigan et al. Journal of Virology 77, 237 LP-244 (2003)) and cVSV-XN (Schnell et al., Journal of virology 70, 2318-2323 (1996)) vectors were kindly provided by Dr. Tiago Abreu-Mota (Thomas Jefferson University, Philadelphia, PA). The chimeric GP38 protein was cloned by first PCR amplifying the human Igk signal sequence with primers GSP49 and GSP53 and GP38 with primers GSP54 and GSP55. This construct was cloned into a pDisplay vector with the addition of an HA tag through In-Fusion® cloning (Takara Bio). The GP38 gene containing the Igk signal sequence was then PCR amplified with primers GSP68 and GSP69, and the RABV-G tail was amplified with primers GSP70 and GSP71. Through In-fusion®, these two PCR products were combined and cloned into a pCAGGS vector. This chimeric GP38 gene was then inserted into the BNSP333 vector using restriction sites BsiWI and NheI, and the plasmid was designated BNSP333-GP38. To produce the GP85 chimeric protein, the MLD gene was PCR amplified from the original CCHFV-coM gene using primers GSP84 and GSP85, and the GP38 chimeric gene was PCR amplified using primers GSP86 and GSP71, excluding the signal sequence. This chimeric GP85 gene was cloned into a pCAGGS vector with In-fusion® cloning and finally cloned into the BNSP333 vector using restriction sites BsiWI and NheI. This resulting plasmid was designated BNSP333-GP85. All CCHFV-coM genes were PCR amplified to have 50 amino acids in the Gc cytoplasmic tail truncated as described in Suda et al. (Archives of Virology 161, 1447-1454 (2016)). Primers GSP03 and GSP20 (GP38+ Gc+) or GSP21 (GP38−Gc+) were used to PCR amplify the CCHFV-coM for the VSV vectors, and GSP06 and GSP07 were used to PCR amplify RABV-G containing the R333E mutation (RVG-333) for the VSV vector. CCHFV-coM was inserted into the VSV vectors using either Mlul and NotI (GP38− Gc+) or Mlul and NheI (GP38+ Gc+) restriction sites. RVG-333 was inserted into the VSV vector containing CCHFV-coM using NotI and NheI restriction sites. The resulting plasmids were designated VSV-AG-CCHFV-coM-RVG (GP38− Gc+) and VSV-AG-CCHFV-coM (GP38+Gc+). The sequences of these three plasmids were confirmed by sequencing using primers GSP08, GSP09, RP591, RP592, RP1325, and RP1327 for the RABV vector and GSP08-GSP19, VPF5, and VP9R for the VSV vectors. Primer sequences are listed in Table 2 below:

TABLE 2
Primer Sequences

| Primer | Direction | Sequence | Use |
|---|---|---|---|
| GSP03 | Forward | 5′- | PCR amplification of CCHFV- |
| | | CGATCTGTTTACGCGTGCCACCA | coM for insertion into VSV |
| | | TGCACATCAGCC-3′ (SEQ ID | vectors. Primer contains MluI |
| | | NO: 6) | restriction site. |
| GSP06 | Forward | 5′- | PCR amplification of RVG-333 |
| | | AGATATCACGCTCGAGGCCACCA | for insertion into VSV vector. |
| | | TGGTTCCTCAGG-3′ (SEQ ID | Primer contains NotI restriction |
| | | NO: 7) | site. |
| GSP07 | Reverse | 5′- | PCR amplification of RVG-333 |
| | | GAAGAATCTGGCTAGCTTACAGT | for insertion into VSV vector. |
| | | CTGGTCTCACCCCC-3′ (SEQ ID | Primer contains NheI restriction |
| | | NO: 8) | site. |
| GSP08 | Reverse | 5′-CTCGCCGGTGATGAAGAACT-3′ | CCHFV-coM sequencing primer. |
| | | (SEQ ID NO: 9) | |
| GSP09 | Forward | 5′-ACCCTGTGAGAAACCTGCTG-3′ | CCHFV-coM sequencing primer. |
| | | (SEQ ID NO: 10) | |
| GSP10 | Reverse | 5′-TTGATCACGCAGTCGGTGAA-3′ | CCHFV-coM sequencing primer. |
| | | (SEQ ID NO: 11) | |
| GSP11 | Forward | 5′-CCTGAAGGCCAGCATCTTCA-3′ | CCHFV-coM sequencing primer. |
| | | (SEQ ID NO: 12) | |
| GSP12 | Reverse | 5′-GCAGTAGGGGCAGATGTTGT-3′ | CCHFV-coM sequencing primer. |
| | | (SEQ ID NO: 13) | |
| GSP13 | Forward | 5′-GGCGATATCCTGGTGGACTG-3′ | CCHFV-coM sequencing primer. |
| | | (SEQ ID NO: 14) | |
| GSP14 | Reverse | 5′-CAGTGTCTGCAGTAAGGGC-3′ | CCHFV-coM sequencing primer. |
| | | (SEQ ID NO: 15) | |
| GSP15 | Forward | 5′-TGCCCTTACTGCAGACACTG-3′ | CCHFV-coM sequencing primer. |
| | | (SEQ ID NO: 16) | |
| GSP16 | Reverse | 5′-ATGTTTCTGGGCTCGGACAG-3′ | CCHFV-coM sequencing primer. |
| | | (SEQ ID NO: 17) | |
| GSP17 | Forward | 5′-TCAACGTGCAGTCCACCTAC-3′ | CCHFV-coM sequencing primer. |
| | | (SEQ ID NO: 18) | |
| GSP18 | Reverse | 5′-TCCTCCTCGCTACAGCTCTT-3′ | CCHFV-coM sequencing primer. |
| | | (SEQ ID NO: 19) | |
| GSP19 | Forward | 5′- | CCHFV-coM sequencing primer. |
| | | AAGAGCTGTAGCGAGGAGGA-3′ | |
| | | (SEQ ID NO: 20) | |
| GSP20 | Reverse | 5′- | PCR amplification of CCHFV- |
| | | GCTAGCTTAGCCTCTGGTTCTCC | coM for insertion into VSV-ΔG |
| | | G-3′ (SEQ ID NO: 21) | vector for surrogate challenge |
| | | | virus. Primer contains NheI |
| | | | restriction site. |
| GSP21 | Reverse | 5′- | PCR amplification of CCHFV- |
| | | GCGGCCGCTTAGCCTCTGGTTCT | coM for insertion into VcoM |
| | | CCG-3′ (SEQ ID NO: 22) | vector for vaccine. Primer |
| | | | contains NotI restriction site. |
| GSP42 | Reverse | 5′-CATAGTCATCTTCATTGA-3′ | Sequencing primer for RVG in |
| | | (SEQ ID NO: 23) | VSV-ΔG-coM-RVG. |
| GSP49 | Forward | 5′- | PCR amplification of signal |
| | | GCCGCCAGTGTGCTGGAATTCGC | sequence for GP38 chimeric |
| | | CACCATGGAGACAGACACA-3′ | gene. |
| | | (SEQ ID NO: 24) | |
| GSP53 | Reverse | 5′- | PCR amplification of signal |
| | | TCTTCAGGTTGTCACCAGTGGAA | sequence for GP38. |
| | | CCTGGAACC-3′ (SEQ ID NO: 25) | |
| GSP54 | Forward | 5′- | PCR amplification of GP38 for |
| | | CACTGGTGACAACCTGAAGATG | GP38 with a signal sequence. |
| | | GAGATCA-3′ (SEQ ID NO: 26) | |
| GSP55 | Reverse | 5′- | PCR amplification of GP38 for |
| | | AACATCGTATGGATAGTCGACGG | GP38 with a signal sequence. |
| | | ACCCGGTGCTGGCCTT-3′ (SEQ ID | |
| | | NO: 27) | |
| GSP66 | Forward | 5′- | Primer for IVT of VSV-N. |
| | | TAATACGACTCACTATAGGGGGA | |
| | | CAGCCTGATGACATTG-3′ (SEQ ID | |
| | | NO: 28) | |
| GSP67 | Reverse | 5′-TCTGGTGCATACAAACCT-3′ | Primer for IVT of VSV-N. |
| | | (SEQ ID NO: 29) | |
| GSP68 | Forward | 5′- | PCR amplification to put the |
| | | CAAAGAATTCCGGAACGTACGG | GP38 with the signal sequence |
| | | CCACCATGGAGACAGACACA-3′ | into a pCAGGS plasmid. |
| | | (SEQ ID NO: 30) | |
| GSP69 | Reverse | 5′- | PCR amplification to put the |
| | | CCGAGGATTCGGACCCGGTGCT | GP38 with the signal sequence |
| | | GGCCTT-3′ | into a pCAGGS plasmid. |
| | | (SEQ ID NO: 31) | |
| GSP70 | Forward | 5′- | PCR amplification of RABV-G |
| | | CACCGGGTCCGAATCCTCGGTTA | 51 amino acids of the |
| | | TCCCCC-3′ | ectodomain (ED51), |
| | | (SEQ ID NO: 32) | transmembrane domain (TM) |
| | | | and cytoplasmic tail (CT) to |
| | | | make the chimeric GP38. |
| GSP71 | Reverse | 5′- | PCR amplification of RABV-G |
| | | GAGGGAAAAAGATCTGCTAGCT | 51 amino acids of the |
| | | TACAGTCTGGTCTCACCCCC-3′ | ectodomain (ED51), |
| | | (SEQ ID NO: 33) | transmembrane domain (TM) |
| | | | and cytoplasmic tail (CT) to |
| | | | make the chimeric GP38. |
| GSP72 | Forward | 5′-CCTCTGCCGACTTGGCACAA-3′ | Primer for qPCR of VSV-N. |
| | | (SEQ ID NO: 34) | |
| GSP73 | Probe | 5′- | Primer-probe for qPCR of VSV- |
| | | CCGGAGGATTGACGACTAATGCA | N. |
| | | CCGCCACAAGGCAG-3′ (SEQ ID | |
| | | NO: 35) | |
| GSP74 | Reverse | 5′-CCGAGCCATTCGACCACATC-3′ | Primer for qPCR of VSV-N. |
| | | (SEQ ID NO: 36) | |
| GSP84 | Forward | 5′- | PCR amplification of MLD to |
| | | CAAAGAATTCCGGAACGTACGAT | create the chimeric GP85. |
| | | GCACATCAGCCTGATGTACGC-3′ | |
| | | (SEQ ID NO: 37) | |
| GSP85 | Reverse | 5′- | PCR amplification of MLD to |
| | | TCTTCAGGTTCCGCTTGCTCCTG | create the chimeric GP85. |
| | | TTGGTGG-3′ | |
| | | (SEQ ID NO: 38) | |
| GSP86 | Forward | 5′- | PCR amplification of chimeric |
| | | GAGCAAGCGGAACCTGAAGATG | GP38 to create the chimeric |
| | | GAGATCATCCTGA-3′ | GP85. |
| | | (SEQ ID NO: 39) | |
| GSP87 | Forward | 5′- | PCR amplification of human |
| | | AATTCCGGAACGTACGGCCACC | furin gene to insert into |
| | | ATGGAGCTGAGG-3′ (SEQ ID | pCAGGS plasmid. |
| | | NO: 40) | |
| GSP88 | Reverse | 5′- | PCR amplification of human |
| | | AAAAAGATCTGCTAGCTTAGAG | furin gene to insert into |
| | | GGCGCTCTGGTC-3′ (SEQ ID | pCAGGS plasmid. |
| | | NO: 41) | |
| RP591 | Forward | 5′- | Sequencing of foreign gene in |
| | | GGAGGTCGACTAAAGAGATCTC | BNSP333 vector. |
| | | ACATAC-3′ (SEQ ID NO: 42) | |
| RP592 | Reverse | 5′- | Sequencing of foreign gene in |
| | | TTCTTCAGCCATCTCAAGATCGG | BNSP333 vector. |
| | | CCAGAC-3′ (SEQ ID NO: 43) | |
| RP1325 | Forward | 5′-GTTATGGTGCCATTAAACCGC | Sequencing of RVG in BNSP333 |
| | | TG-3′ (SEQ ID NO: 44) | vector. |
| RP1327 | Reverse | 5′-TCTCCAGGATCGAGCATCTT-3′ | Sequencing of RVG in BNSP333 |
| | | (SEQ ID NO: 45) | vector. |
| VP5F | Forward | 5′-GCGTGGGTCCTGGATTCTAT-3′ | Sequencing of foreign gene in |
| | | (SEQ ID NO: 46) | VSV vector |
| VP9R | Reverse | 5′-ATCGAGGGAATCGGAAGAGA | Sequencing of foreign gene in |
| | | AT-3′ (SEQ ID NO: 47) | VSV vector |

### Recovery of Recombinant Viruses

Recombinant RABV and VSV vaccines were recovered as described in Harty et al. (Journal of molecular microbiology and biotechnology 3, 513-517 (2001)) and Schnell et al. (The EMBO journal 13, 4195-4203 (1994)). Briefly, X-tremeGENE 9 (MilliporeSigma®) in Opti-MEM (Gibco®) was used to co-transfect the respective full-length viral cDNA along with the plasmids encoding RABV N, P, and L or VSV N, P, and L proteins, with the addition of RABV G for the VSV surrogate challenge virus and pCAGGs plasmids expressing T7 RNA polymerase in 293T cells in poly-1-lysine coated 6-well plates. The supernatants of RABV transfected cells were harvested every 3 days, and VSV transfected cell supernatants were harvested every 2 days. Presence of infectious RABV was detected by immunostaining for RABV N with 1:200 dilution of fluorescein isothiocyanate (FITC) anti-RABV N monoclonal globulin (Fujirebio®, product #800-092) or for virus-induced cytopathic effect (CPE) in the case of VSV.

### Viral Production and Titering

GP38+ Gc−, GP38− Gc+, GP38+ Gc+, Filorab 1 (generous gift of Dr. Drishya Kurup, Thomas Jefferson University, Philadelphia, PA), BNSP333, VSV-GFP (plasmid provided by Dr. Tiago Abreu-Mota), VSV-AG-RABV-G and SPBN viruses were grown and titered on Vero cells. Specifically, Vero cells were cultured with VP-SFM (Gibco®) supplemented with 1% P/S, 2X GlutaMAX™ (Gibco®) and 10 mM HEPES buffer (Corning®) and infected with a multiplicity of infection (MOI) of 0.01 for Filorab1, BNSP333, and VSV-GFP and 0.001 for GP38+ Gc−, GP38− Gc+, and GP38+ Gc+. GP38+ Gc+ to be used in the surrogate challenge model was grown on BSR cells in DMEM supplemented with 5% FBS and 1% P/S, infected at MOI 0.001. VSV-AG-RABV-G and SPBN were grown on BEAS-2B cells in OptiPRO™ SFM (Gibco™), supplemented with 1% P/S, 2X GlutaMAX™ (Gibco®) and 10 mM HEPES buffer (Corning®), and infected with a multiplicity of infection (MOI) of 0.01. Viruses were harvested every 3 days with VP-SFM media replacement until viral titers started to decrease for RABVs or until 80% cytopathic effect was detected for VSVs. RABV titering was performed by limiting dilution focus-forming assay using FITC anti-RABV N monoclonal globulin (Fujirebio®; catalogue number: 800-092) as described in Pulmanausahakul et al. (Journal of Virology 82, 2330 LP-2338 (2008)). VSV titers were determined by plaque forming assay using 2% methyl cellulose overlay.

### Purification and Virus Inactivation

To produce inactivated GP38+ Gc−, GP38− Gc+, GP38+ Gc+, and Filorab1 vaccines, viral supernatant was concentrated, sucrose purified, and inactivated. Briefly, viral supernatants with the highest titers were pooled for each virus and concentrated at least 5x in an Amicon® 300 mL stirred cell concentrator using a 500 kDa exclusion PES membrane (MilliporeSigma®). Concentrated supernatants were then overlaid onto a 20% sucrose cushion and centrifuged at 76,755×g for 2 hrs. Virions pellets were resuspended in TEN buffer (100 mM Tris base, 50 mM NaCl, 2 mM EDTA in ddH2O) with 2% sucrose and incubated overnight (O.N.) at 4° C. β-propiolactone (BPL) (MilliporeSigma®) was added at a 1:2000 dilution for inactivation. Samples were left at 4° C. O.N. shaking and then incubated the following day at 37° C. for 30 min to hydrolyze the BPL. Virus inactivation was confirmed. Briefly, supernatant inoculated with 10 μg of inactivated virions was passaged in T25 flasks of Vero cells; cells were fixed and stained with FITC anti-RABV N or monitored for cytopathic effect.

### Immunofluorescence

3E5 Vero cells were seeded on glass coverslips in a 12-well plate and infected the next day at an MOI of 0.01 with the respective viruses. After 72 hrs (RABV viruses) or 24 hrs (VSV viruses), cells were washed in 1X DPBS and fixed for 10 mins in 2% paraformaldehyde (PFA) in 1X DPBS for surface staining. Those slides to be used for intracellular staining were then fixed for an additional 15 mins in 2% PFA with 0.1% Triton™ X-100 (Sigma-Aldrich®). Subsequently, cells were washed 2-3 times with 1X DPBS and blocked in 1X DPBS with 5% FBS for 1 hr at room temperature or overnight at 4° C. Cells were then probed for 1 hr at room temperature with primary antibodies in 1X DPBS with 1% FBS, specifically, anti-RABV-G 4C12 at 4 μg/mL, with either anti-Gc 11E7 at 3.2 μg/mL or anti-GP38 13G8 at 2.4 μg/mL. Cells were washed once with 1X DPBS and incubated with 2.5 μg/mL of anti-mouse AF568 and 2.5 μg/mL of anti-human AF647 in 1X DPBS with 1% FBS for 45 mins at room temperature. Cells were then washed 5 times with 1X DPBS, mounted onto slides using mounting media containing 4′,6-diamidino-2-phenylindole (DAPI) (ProLong™ Glass Antifade Mountant, Invitrogen™ catalog number: P36980), and stored O.N. at room temperature in the dark. Slides were visualized the next day using a Nikon Ti-E microscope with Nikon AIR Laser Scanning confocal camera with the Plan Fluor 40x/1.3 objective lens on the NIS-Elements C software for multi-dimensional experiment acquisition and analysis at 23° C. Color channels were processed (channels separated for individual images and merged for merged images) using ImageJ software (OSS NIH).

### Glycoprotein FACS Analysis

A total of 8E5 Vero cells for RABVs or 3e5 Vero cells for VSVs were seeded in 6-well plates. The following day, cells were infected with RABVs at MOI 10 for 48 hrs or left uninfected (control). Two days later, cells were infected with VSVs at MOI 5 for 8 hrs. Medium was then aspirated, and cells were washed once with 1X DPBS. Cellstripper® (Corning™, catalog number 25-056-Cl) was added to each well for 5-10 min to remove the cells from the well. Cells were then transferred to 15 mL conical tubes and centrifuged at 400×g for 5 min. Cells were resuspended in 100 μL per 8E5 cells of 2% PFA in 1×PBS, seeded in a 96-well round bottom plate with 8E5 cells per well, and fixed for 10 min. Cells were centrifuged at 250×g for 3 min and washed three times in 200 μL FACS buffer (10% FBS and 0.05% NaN₃) per well. Cells were stained in 100 μL of primary antibody mixture containing anti-RABV-G 4C12 at 4 μg/mL and either anti-Gc 11E7 at 3.2 μg/mL or anti-GP38 13G8 at 2.4 μg/mL in FACS buffer O.N. at 4° C. The next day, cells were washed twice with 200 μL FACS buffer and then stained with 100 μL of secondary antibody mixture containing goat anti-mouse BV510 at 0.2 μg/100 μL and goat anti-human AF647 at 2.5 μg/mL in FACS buffer for 2 hrs at room temperature. Cells were then washed 3 times in 200 μL FACS buffer and transferred to FACS tubes in a total of 400 μL FACS buffer. Cells were analyzed for GFP emission to detect GFP expression (i.e., VSV-GFP infection) in the FITC channel, BV510 emission to detect CCHFV-Gc or GP38 in the BV510 channel, and AF647 emission to detect RABV-G in the allophycocyanin (APC) channel using a BD FACSCelesta™ Cell Analyzer. Data analysis was performed using FlowJo software (Treestar, Ashland, OR).

### SDS PAGE Protein Gel and Western Blot

Sucrose purified virus particles and purified CCHFV glycoproteins were denatured with Urea Sample Buffer (125 mM Tris-HCl [pH 6.8], 8 M urea, 4% sodium dodecyl sulfate, 0.02% bromophenol blue) and reduced with 2-mercaptoethanol (CAS No. 60-24-2, Millipore Sigma®) and boiling at 95° C. for 10 min. However, samples to be probed with any of the anti-CCHFV antibodies were left unreduced, as these antibodies are conformational. 1 μg of samples for total protein analysis were resolved on a 10% SDS-PAGE gel and stained O.N. with SYPRO™ Ruby Protein Gel Stain (ThermoFisher Scientific). 1 μg of samples for western blot analysis were resolved on a 10% SDS-PAGE gel and transferred onto a nitrocellulose membrane in Towbin buffer (192 mM glycine, 25 mM Tris, 20% methanol). Blots were then blocked in 5% milk dissolved in PBS-T (0.05% Tween® 20 [MilliporeSigma®]) at room temperature for 1 hr. Next, membranes were incubated with primary antibody O.N. at 4° C. Antibodies were made in a solution of 5% bovine serum albumin (BSA) in PBS. Anti-Gc 11E7 was used at a dilution of 320 ng/mL, anti-GP38 13G8 was used at a dilution of 240 ng/ml, and anti-RABV-G 4C12 was used at 2 μg/mL dilution. The next day the blots were washed with PBS-T and incubated with horseradish peroxidase (HRP)-conjugated anti-mouse or human IgG at 1:40,000 dilution in PBS-T for blots probed with 11E7, 1:20,000 dilution in PBS-T for blots probed with 13G8 or 1:20,000 in PBS-T for blots probed with 4C12. Proteins were detected with SuperSignal West Dura Chemiluminescent substrate (Pierce®) and imaged on the FluorChem R system (proteinsimple®).

### Multi-Step and One-Step Growth Curves

Vero E6 cells were seeded in 6-well plates at 7E5 cells/well. The following day, cells were checked for 70% confluency and then infected in serum free medium at MOI 0.01 for multi-step growth curves or MOI 10 for one-step growth curves. After two hours of incubation, the media was aspirated, and the infected cells were washed 2× with 1X DPBS (Corning®). DMEM supplemented with 5% FBS and 1% P/S was added to each well, and the first sample of 200 μL was taken from each well. Samples were taken every 24 hrs until 120 hrs post-infection for RABVs and at 2, 4, 6, 8, 12, 24, 36, and 48 hrs post-infection for VSVs. Each viral sample was titered in triplicate as described above in the Viral production and titering section.

### Immunizations

Groups of five 6- to 10-week-old male and female C57BL/6 mice were immunized intramuscularly (I.M.) with 10 μg BPL-inactivated virus (see FIG. 23A for dose schedule) formulated alone in PBS or with the addition of Synthetic Monophosphoryl Lipid A (MPLA), 3D (6A)-PHAD, in a squalene-in-oil emulsion (PHAD-SE), at a dose of 5 μg PHAD and 2% SE. Each immunization was administered as a total of 100 μL, with 50 μL injected in each hind leg muscle. Serum was collected through retro-orbital bleeds performed under isoflurane anesthesia on days 0, 14, 28, 35, and 42, with the final bleed on day 56.

### Production of ELISA Antigens

RABV-G antigen was produced as described in Blaney et al. (PLOS Pathogens 9, e1003389-e1003389 (2013)). Briefly, BEAS-2B cells were infected with VSV-AG-GFP-RABV-G (for RABV vaccines) or SPBN (for VSV vaccines) in Opti-PRO (Gibco®). Viral supernatants were concentrated and purified as described above in the purification section. After sucrose purification, viral pellets were resuspended in TEN buffer (100 mM NaCl, 100 mM Tris, 10 mM EDTA pH7.6) containing 2% OGP (Octyl-β-D-glucopyranoside) detergent and incubated for 30 min at room temperature while shaking. This mixture was centrifuged at 3000×g for 10 min, supernatant collected and further centrifuged at 25,000×g for 90 min. Supernatant was collected and analyzed for presence of antigen via western blot with anti-RABV-G antibody.

CCHFV-Gc HA-tagged antigen was prepared as previously described for other HA-tagged antigens (Kurup et al., Journal of Virology 89, 144-154 (2015)). Subconfluent T175 flasks of 293 T cells that were poly-1-lysine coated were transfected with a eukaryotic expression vector (pDisplay) encoding for each individual CCHFV glycoprotein with the cleavage sites and transmembrane regions removed, specifically amino acids 1040 to 1631 of CCHFV-M, fused to a C-terminal hemagglutinin (HA) peptide. Supernatant was collected one week after transfection, clarified by centrifugation, and filtered through a 0.45 μm filter before being loaded onto an equilibrated anti-HA agarose column (Pierce) containing either a 2.5 mL or 5 mL agarose bed volume. The supernatant was allowed to bind to the column overnight at 4° C. The following day, the column was washed with 10-bed volumes of PBS-T, and bound HA-tagged glycoprotein was eluted with 5-10 mL of 0.4 mg/mL HA peptide in PBS. Fractions were collected and analyzed for the presence of Gc glycoprotein through western blot with CCHFV-Gc 11E7 antibody. Peak fractions were pooled and dialyzed against PBS in 10,000 molecular weight cutoff dialysis cassettes (MWCO) (Thermo Scientific™) to remove excess HA peptide. After dialysis, the protein was quantified by nanodrop 2000c spectrophotometer and/or bicinchoninic acid (BCA) assay. Halt™ Protease Inhibitor Cocktail (Thermo Scientific™, catalog number: 78430) was added for a final concentration of 1X and sodium azide (NaN₃) added for a final concentration of 0.05% before freezing the protein in small aliquots at −80° C.

CCHFV-GP38 Strep-tagged antigen was prepared from an enhanced expression vector (pEEV) containing the sequence for CCHFV-GP85 strain IbAr10200 from amino acids 22 to 515, with a N-terminal FLAG and His tag and a C-terminal Strep-Tag II (referred to as pEEV-HisFlag-GP85-10200-Strep) (generously provided by Dr. Eric Bergeron at the Centers for Disease Control, Atlanta, GA). The plasmid pLEX307-FURIN-puro (ID #158460), containing the human furin gene was ordered from AddGene. This gene was then PCR amplified with primers GSP87 and GSP88 and cloned into a pCAGGS vector through In-Fusion™ cloning. 293F cells were grown in FreeStyle™ 293 Expression Medium (Gibco®) with 2X Glutamax (Gibco®) and seeded at 3×10⁶ cells/mL in Erlenmeyer flasks. The next day, cells were transfected using FectoPRO® (Polyplus transfection™) transfection reagent following the reagent manual with slightly altered conditions. The pEEV-HisFlag-GP85-10200-Strep and pLEX307-FURIN-puro plasmids were transfected at a ratio of 4:1 in a total of 0.8 μg plasmid DNA for each ml of culture. This co-transfection with the furin plasmid was to ensure that the MLD was cleaved from GP38. Media for the transfection complexes was 1/10 of the total culture volume and 1.5 μL of FectoPro reagent was used per μg of DNA. 4 hours after transfection, FectoPRO® booster was added in an equivalent amount to that of DNA (i.e., 0.8 μg/mL DNA=0.8 μL FectoPRO® booster/mL). Cells were incubated until cell viability sharply declined, typically around 3 days post transfection. The supernatant was then harvested, spun down for 30 mins at 4000 × g and filtered through a 0.45 uM filter before being loaded onto a column with a 2 mL bed volume of Strep-Tactin®XT resin (IBA Lifesciences). The supernatant was allowed to bind to the column overnight at 4° C. The following day, the column was washed with 5 column bed volumes of 1X Buffer W (IBA Lifesciences) and then eluted with 6X 0.5 column bed volumes of 1X Buffer BXT (IBA Lifesciences), collected as 0.5 mL fractions. Fractions were analyzed for the presence CCHFV-GP38 through western blot with CCHFV-Gc 13G8 antibody. The protein was quantified by nanodrop 2000c spectrophotometer and/or bicinchoninic acid (BCA) assay. Halt™ Protease Inhibitor Cocktail (Thermo Scientific™, catalog number: 78430) was added for a final concentration of 1X and sodium azide (NaN₃) added for a final concentration of 0.05% before freezing the protein in small aliquots at −80° C.

### Enzyme-Linked Immunosorbent Assay (ELISA)

Individual mouse serum was analyzed by ELISA for the presence of IgG specific to CCHFV-GP38,-Gc, and RABV-G. Antigens were diluted in coating buffer (15 mM Na2CO₃, 35 mM NaHCO₃[pH 9.6]) at a concentration of 100 ng/well (1 ng/μL) for GP38, 150 ng/well (1.5 ng/μL) for Gc, and 50 ng/well (0.5 ng/μL) for RABV-G, and then 100 μL was added to each well of 96-well immulon 4HBX plates (Nunc®) and incubated O.N. at 4° C. The following day, plates were washed three times with PBS-T (0.05% Tween 20 in 1×PBS), blocked for 2 hrs (5% milk in PBS-T), and washed again three times with PBS-T. Sera or control mAbs were diluted in three-fold serial dilutions (starting with a 1:50 dilution or higher dilutions of 1:150, 1:450, or 1:1350 for sera that did not reach endpoint titer) down the plate in 1×PBS with 0.5% BSA and incubated O.N. at 4° C. Plates were then washed three times with PBS-T and 100 μL secondary antibody HRP conjugated goat anti-mouse IgG Fc at a concentration of 50 ng/ml for GP38 and Gc, and 25 ng/mL for RABV-G in PBS-T was added to each well and incubated for 2 hrs at room temperature. For isotype subclass ELISAs, the appropriate secondary antibody was used at the same concentration as the IgG Fc-specific secondary antibody. Then plates were washed three times with PBS-T, and 200 μL of o-phenylenediamine dihydrochloride (OPD) substrate (ThermoFisher®) was added and left incubating for 15 min for GP38 and Gc and 13 min for RABV-G. The reaction was stopped by adding 50 μL of 3M Sulfuric acid (H2SO4). Optical density was determined at 490 nm (OD490) and 630 nm (OD630) and delta values calculated subtracting the background OD630 readings from the OD490 readings. ELISA data was analyzed with GraphPad Prism 8 using a sigmoidal nonlinear fit (4PL regression curve) model to determine the half maximal Effective Concentration (EC₅₀) serum or antibody titer. An accurate EC₅₀ value cannot be calculated without a full curve, therefore samples without a proper curve are considered to have no detectable antibodies against that antigen and have a reported EC₅₀ of 1. Isotype ratios were calculated by taking either the IgG2c or IgG2b EC₅₀ value, dividing it by the IgG1 EC₅₀ value. For those samples where there was no detectable IgG1 antibodies, no isotype ratio could be calculated. Positive controls (when available) for each assay were as follows: α-CCHFV-GP38 13G8 for IgG Fc and IgG2b GP38 ELISAs; α-CCHFV-GP38 10E11 for IgG1 GP38 ELISAs; α-CCHFV-Gc 11E7 for IgG Fc Gc ELISAs; α-RABV-G 1C5 for IgG Fc RABV-G ELISAS.

### Surrogate CCHFV Challenge Virus Pathogenicity

Groups of five 8-10-week-old male interferon α/β receptor 1 knockout (IFNAR^{-/-}) mice were infected with either 5e5, 7.5e5 or 1e6 pfu of GP38+ Gc+ virus I.P. (200 μL total) to determine the parameters needed for use as a challenge model. The virus was diluted in PBS for all doses. Mice were weighed daily and monitored for signs of disease until day 14 post-infection. Mice that lost more than 20% of their starting weight or appeared moribund were humanely euthanized. Blood was collected at days 0, 4, and 14 to be used for in a VSV-N qPCR to look for viremia.

Surrogate CCHFV challenge model in mice

Groups of five 8- to 10-week-old male and female IFNAR^{-/-} mice were immunized I.M. with 10 μg of BPL inactivated GP38+ Gc− or FR1 vaccines adjuvanted with 5 μg PHAD in 2% SE at days 0 and 28 (FIG. 27A). On day 65, mice were injected with 5e5pfu of GP38+ Gc+ diluted in PBS as determined above. Mice were sacrificed: (1) when weight loss reached ≥20% or (2) if severe clinical signs of disease were observed. Terminal bleeding was collected upon sacrifice when possible. Mice were bled at days 0, 4, and 14 to look for viremia in a VSV-N qPCR.

### RNA Extraction

50 μL of whole blood was added to 300 μL of TRIzol LS Reagent (Life Technologies) and 50 μL of DPEC water, or 250 μL of virus supernatant was added to 750 μL of TRIzol LS Reagent. The protocol for RNA extraction of biological fluids with TRIzol LS Reagent was used up to the phase separation step. Then the protocol from the PureLink RNA Mini Kit (Ambion) was used for the remainder of the extraction. A NanoDrop (Fisher) was used to measure the concentration and quality (260/280 ratios) of extracted RNA.

Measuring Surrogate Challenge Virus Viremia via quantitative Real-Time polymerase chain reaction (qPCR)

First, VSV-N RNA was prepared to act as a standard for the qPCR. RNA was isolated from GP38+ Gc+ virus and cDNA produced using the One-Step RT PCR (SuperScript IV, Thermo Fisher Scientific) with primers GSP66 and GSP67. This cDNA was used to produce RNA standards via in-vitro transcription using the MEGAscript® T7 Kit (Invitrogen™) followed by the MEGAclear™ Transcription Clean-Up Kit (Invitrogen™). The qPCR was then run following the protocol for TaqMan Fast Virus 1 Step Master Mix reagent (ThermoFisher), using 5 μL of RNA per reaction, primers GSP72 and GSP74, and probe GSP73 with a 60° C. annealing temperature. Any day 0 samples showing detectable viral RNA were considered contaminated and not reported. Full primer and probe sequences are listed in Table 2.

### Wildtype CCHFV Challenge in IFNAR-Mice

Mice were challenged with 1000pfu of CCHFV strain IbAr10200 by intraperitoneal (i.p.) route as previously described75. Virus was diluted in a total volume of 0.1 ml of PBS (Gibco). All mice were injected i.p. with a total of 2.5 mg of anti-IFNAR 1 (mAb-5A3; Leinco Technologies Inc.) diluted in PBS 24 hours before (2.0 mg) and 24 hours after infection (0.5 mg) in a total volume of 0.2 ml. Mice were observed at least daily and weighed for the first 10 days daily and then every 3 days.

### Wildtype CCHFV FRNT

Mouse sera were serially diluted 1:2 in serum-free DMEM then incubated with rCCHFV-ZsGreen virus for 1 hour on ice. The mixture was inoculated onto wells of HuH-7 cells and incubated for 1 hour at 37° C. with 5% CO₂. Cells were then supplemented with D10 and incubated until 48 hours post infection. Relative fluorescence of each well was measured on a GFP plate reader. Wells inoculated with rCCHFV-ZsGreen virus only served as the control for maximum fluorescence, and wells inoculated with serum-free DMEM without virus served as the control for background fluorescence. Percent virus neutralization was calculated from the percent of fluorescence reduction from serum plus virus wells compared to virus only wells. IC50 values were determined using a four parameter, variable slope, nonlinear regression model in GraphPad PRISM.

### Rapid Fluorescent Focus Inhibition Test (RFFIT)

RFFIT neutralization assay was performed as previously described76. Briefly, serum was heat inactivated at 56° C. for 30 mins. NA cells were seeded at 3E4 cells per well in a 96-well plate. 2 days later, serum samples were diluted in a 2-fold dilution series in Opti-MEM in 96-well plates at a starting dilution of 1:40 (unless stated otherwise). The US standard rabies immune globulin (WHO Standard) was used at a starting dilution of 2IU/mL. A dilution of CVS-11 previously determined to produce 90% infection was added to each well with either sera or the WHO Standard and incubated for 1 hr at 34° C. The media in the plates with the NA cells was then replaced by the sera/virus mixture and incubated for 2 hrs at 34° C. This media was aspirated, and fresh Opti-MEM was added. Plates were incubated for 22 hrs at 34° C. and then fixed with 80% acetone and stained with FITC-conjugated anti-RABV-N antibody for at least 4 hours. The Reed-Muench method was used to calculate 50% endpoint titers, which were subsequently converted to international units (IU) per milliliter through comparison to the WHO standard.

### Statistical Analysis

All statistical analysis was performed using GraphPad Prism 9 on log transformed data. For growth curves, each time point was compared to the parental vector control using the ordinary one-way ANOVA with the Tukey Multiple Comparison Test. The Mann Whitney test was used for comparison within two groups at each timepoint for all ELISA EC₅₀ data and IU/mL RFFIT data. For groups analysis at each time point of ELISA EC₅₀ titers, IU/mL RFFIT data, and qPCR viral RNA copies, an ordinary one-way ANOVA was used with a post-Hoc analysis using Tukey Multiple Comparison Test with a 95% confidence interval. To look at the differences in group average weight change over time for the surrogate challenge virus, a two-way ANOVA was used with Tukey's Multiple Comparisons Test. A two-way ANOVA was used with a Dunnett multiple comparisons test to compare differences in weight loss over time to the control female PBS group for the WT CCHFV challenge. The log-rank Mantel-Cox test was performed to compare differences in survival to the control female PBS group. All mouse studies were performed with groups of 5 mice unless otherwise stated. While groups of 5 are sufficient for statistical analysis, two groups for each vaccine (one male and one female) were tested in each challenge study, further strengthening the results. Additionally, the WT CCHFV challenge model is 100% lethal and power analysis showed that a 20% difference in survival can be detected with a p value of 0.05.

### Enumerated Embodiments

In some aspects, the present invention is directed to the following non-limiting embodiments:

Embodiment 1: A composition comprising a recombinant vector from an attenuated rabies virus comprising a nucleotide sequence encoding at least one Crimean-Congo hemorrhagic fever virus (CCHFV) glycoprotein and a mucin-like domain.

Embodiment 2: The composition of Embodiment 1, wherein the at least one CCHFV glycoprotein is GP38, or an antigenic fragment thereof

Embodiment 3: The composition of Embodiment 1, wherein the nucleotide sequence encoding the recombinant rabies vector has at least 80% sequence identity with SEQ ID NO: 1.

Embodiment 4: The composition of Embodiment 1, wherein the nucleotide sequence encoding the recombinant rabies vector has at least 85%, at least 90%, at least 95%, at least 98%, at least 99% or 100% sequence identity with SEQ ID NO: 1.

Embodiment 5: The composition of Embodiment 2, wherein the nucleotide sequence encoding GP38 has at least 80% sequence identity with SEQ ID NO: 2.

Embodiment 6: The composition of Embodiment 2, wherein the nucleotide sequence encoding GP38 has at least 85%, at least 90%, at least 95%, at least 98%, at least 99% or 100% sequence identity with SEQ ID NO: 2.

Embodiment 7: The composition of Embodiment 2, wherein the protein sequence of GP38 has at least 80% sequence identity with SEQ ID NO: 3.

Embodiment 8: The composition of Embodiment 2, wherein the protein sequence of GP38 has at least 85%, at least 90%, at least 95%, at least 98%, at least 99% or 100% sequence identity with SEQ ID NO: 3.

Embodiment 9: The composition of Embodiment 1, wherein the nucleotide sequence encoding the mucin-like domain has at least 80% sequence identity with SEQ ID NO: 4.

Embodiment 10: The composition of Embodiment 1, wherein the nucleotide sequence encoding the mucin-like domain has at least 85%, at least 90%, at least 95%, at least 98%, at least 99% or 100% sequence identity with SEQ ID NO: 4.

Embodiment 11: The composition of Embodiment 1, wherein the protein sequence of the mucin-like domain has at least 80% sequence identity with SEQ ID NO: 5.

Embodiment 12: The composition of Embodiment 1, wherein the protein sequence of the mucin-like domain has at least 85%, at least 90%, at least 95%, at least 98%, at least 99% or 100% sequence identity with SEQ ID NO: 5.

Embodiment 13: The composition of Embodiment 1, wherein the composition is therapeutically effective.

Embodiment 14: The composition of Embodiment 1 wherein the composition is immunogenic.

Embodiment 15: The composition according to Embodiment 1, further comprising a pharmaceutically acceptable excipient.

Embodiment 16: The composition according to Embodiment 15, wherein the pharmaceutically acceptable excipient is an adjuvant.

Embodiment 17: An isolated virion prepared from a host cell infected with the recombinant vector of Embodiment 1.

Embodiment 18: A method of conditioning an immune response protective against a CCHFV virus in a subject, the method comprising administering to the subject a recombinant vector from an attenuated rabies virus comprising a nucleotide sequence encoding at least one CCHFV glycoprotein and a mucin-like domain.

Embodiment 19: The method of Embodiment 18, wherein the at least one CCHFV glycoprotein is GP38.

Embodiment 20: The method of Embodiment 18, wherein the nucleotide sequence encoding the recombinant rabies vector has at least 80% sequence identity with SEQ ID NO: 1.

Embodiment 21: Te method of Embodiment 18, wherein the nucleotide sequence encoding the recombinant rabies vector has at least 85%, at least 90%, at least 95%, at least 98%, at least 99% or 100% sequence identity with SEQ ID NO: 1.

Embodiment 22: The method of Embodiment 19, wherein the nucleotide sequence encoding GP38 has at least 80% sequence identity with SEQ ID NO: 2.

Embodiment 23: The method of Embodiment 19, wherein the nucleotide sequence encoding GP38 has at least 85%, at least 90%, at least 95%, at least 98%, at least 99% or 100% sequence identity with SEQ ID NO: 2.

Embodiment 24: The method of Embodiment 19, wherein the protein sequence of GP38 has a least 80% sequence identity with SEQ ID NO: 3.

Embodiment 25: The method of Embodiment 19, wherein the protein sequence of GP38 has at least 85%, at least 90%, at least 95%, at least 98%, at least 99% or 100% sequence identity with SEQ ID NO: 3.

Embodiment 26: The method of Embodiment 18, wherein the nucleotide sequence encoding the mucin-like domain has at least 80% sequence identity with SEQ ID NO: 4.

Embodiment 27: The method of Embodiment 18, wherein the nucleotide sequence encoding the mucin-like domain has at least 85%, at least 90%, at least 95%, at least 98%, at least 99% or 100% sequence identity with SEQ ID NO: 4.

Embodiment 28: The method of claim 18, wherein the protein sequence of the mucin-like domain has at least 80% sequence identity with SEQ ID NO: 5.

Embodiment 29: The composition of Embodiment 18, wherein the protein sequence of the mucin-like domain has at least 85%, at least 90%, at least 95%, at least 98%, at least 99% or 100% sequence identity with SEQ ID NO: 5.

Embodiment 30: The method of Embodiment 18, wherein the composition is therapeutically effective.

Embodiment 31: The method of Embodiment 18 wherein the composition is immunogenic.

Embodiment 32: The method of Embodiment 18, wherein the immune response is directed against a CCHFV glycoprotein.

Embodiment 33: The method of claim 18, wherein the immune response is directed against a CCHFV glycoprotein and a rabies protein.

Embodiment 34: The method of Embodiment 32, wherein the immune response directed against the CCHFV glycoprotein primarily comprises non-neutralizing antibodies.

Embodiment 35: A method of treating a subject infected with a CCHFV virus, comprising administering to the subject a composition comprising a recombinant vector from an attenuated rabies virus that expresses at least one CCHFV immunogenic protein and a mucin-like domain, wherein said composition induces an effective immune response against one or both of said viruses wherein the at least one immunogenic CCHFV protein comprises the amino acid sequence of SEQ ID NO: 3.

Embodiment 36: A CCHFV ribonucleic acid (RNA) polynucleotide vaccine, comprising at least one RNA having an open reading frame encoding at least one CCHFV glycoprotein or an immunogenic fragment thereof and a mucin-like domain, and a pharmaceutically acceptable excipient.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A composition comprising a recombinant vector from an attenuated rabies virus comprising a nucleotide sequence encoding at least one Crimean-Congo hemorrhagic fever virus (CCHFV) glycoprotein and a mucin-like domain.

2. The composition of claim 1, wherein the at least one CCHFV glycoprotein is GP38, or an antigenic fragment thereof.

3. The composition of claim 1, wherein at least one of the following applies:
(a) the nucleotide sequence encoding the recombinant rabies vector has at least 80% sequence identity with SEQ ID NO: 1,
(b) the nucleotide sequence encoding the recombinant rabies vector has at least 85%, at least 90%, at least 95%, at least 98%, at least 99% or 100% sequence identity with SEQ ID NO:
1,
(c) the nucleotide sequence encoding the mucin-like domain has at least 80% sequence identity with SEQ ID NO: 4,
(d) the nucleotide sequence encoding the mucin-like domain has at least 85%, at least 90%, at least 95%, at least 98%, at least 99% or 100% sequence identity with SEQ ID NO: 4,
(e) the protein sequence of the mucin-like domain has at least 80% sequence identity with SEQ ID NO: 5,
(f) the protein sequence of the mucin-like domain has at least 85%, at least 90%, at least 95%, at least 98%, at least 99% or 100% sequence identity with SEQ ID NO: 5,
(g) the composition is therapeutically effective,
(h) the composition is immunogenic, or
(i) the composition further comprises a pharmaceutically acceptable excipient.

4. (canceled)

5. The composition of claim 2, wherein at least one of the following applies:
(a) the nucleotide sequence encoding GP38 has at least 80% sequence identity with SEQ ID NO: 2,
(b) the nucleotide sequence encoding GP38 has at least 85%, at least 90%, at least 95%, at least 98%, at least 99%, or 100% sequence identity with SEQ ID NO: 2,
(c) the protein sequence of GP38 has at least 80% sequence identity with SEQ ID NO: 3,
(d) the protein sequence of GP38 has at least 85%, at least 90%, at least 95%, at least 98%, at least 99%, or 100% sequence identity with SEQ ID NO: 3.

6-15. (canceled)

16. The composition according to claim 3, wherein the composition further comprises the pharmaceutically acceptable excipient, and wherein the pharmaceutically acceptable excipient is an adjuvant.

17. An isolated virion prepared from a host cell infected with the recombinant vector of claim 1.

18. A method of conditioning an immune response protective against a CCHFV virus in a subject, the method comprising administering to the subject a recombinant vector from an attenuated rabies virus comprising a nucleotide sequence encoding at least one CCHFV glycoprotein and a mucin-like domain.

19. The method of claim 18, wherein the at least one CCHFV glycoprotein is GP38.

20. The method of claim 18, wherein at least one of the following applies:
(a) the nucleotide sequence encoding the recombinant rabies vector has at least 80% sequence identity with SEQ ID NO: 1,
(b) the nucleotide sequence encoding the recombinant rabies vector has at least 85%, at least 90%, at least 95%, at least 98%, at least 99% or 100% sequence identity with SEQ ID NO: 1,
(c) the nucleotide sequence encoding the mucin-like domain has at least 80% sequence identity with SEQ ID NO: 4,
(d) the nucleotide sequence encoding the mucin-like domain has at least 85%, at least 90%, at least 95%, at least 98%, at least 99% or 100% sequence identity with SEQ ID NO: 4,
(e) the protein sequence of the mucin-like domain has at least 80% sequence identity with SEQ ID NO: 5,
(f) the protein sequence of the mucin-like domain has at least 85%, at least 90%, at least 95%, at least 98%, at least 99% or 100% sequence identity with SEQ ID NO: 5,
(g) the composition is therapeutically effective,
(h) the composition is immunogenic,
(i) the immune response is directed against a CCHFV glycoprotein, or
(j) the immune response is directed against a CCHFV glycoprotein and a rabies protein.

21. (canceled)

22. The method of claim 19, wherein at least one of the following applies:
(a) the nucleotide sequence encoding GP38 has at least 80% sequence identity with SEQ ID NO: 2,
(b) the nucleotide sequence encoding GP38 has at least 85%, at least 90%, at least 95%, at least 98%, at least 99% or 100% sequence identity with SEQ ID NO: 2,
(c) the protein sequence of GP38 has a least 80% sequence identity with SEQ ID NO: 3,
(d) the protein sequence of GP38 has at least 85%, at least 90%, at least 95%, at least 98%, at least 99% or 100% sequence identity with SEQ ID NO: 3.

23-33. (canceled)

34. The method of claim 20, wherein the immune response is directed against the CCHFV glycoprotein, and wherein the immune response directed against the CCHFV glycoprotein primarily comprises non-neutralizing antibodies.

35. A method of treating a subject infected with a CCHFV virus, comprising administering to the subject a composition comprising a recombinant vector from an attenuated rabies virus that expresses at least one CCHFV immunogenic protein and a mucin-like domain, wherein said composition induces an effective immune response against one or both of said viruses wherein the at least one immunogenic CCHFV protein comprises the amino acid sequence of SEQ ID NO: 3.

36. A CCHFV ribonucleic acid (RNA) polynucleotide vaccine, comprising at least one RNA having an open reading frame encoding at least one CCHFV glycoprotein or an immunogenic fragment thereof and a mucin-like domain, and a pharmaceutically acceptable excipient.
